(19) United States
(12) Patent Application Publication
(10) Pub. No.: US 2024/0101997 A1
(43) Pub. Date: Mar. 28, 2024

(54) DEVICE AND METHOD FOR PRODUCING EXTRACELLULAR VESICLES

(71) Applicant: The University of Chicago, Chicago, IL (US)

(72) Inventors: Bozhi Tian, Chicago, IL (US); Lingyuan Meng, Chicago, IL (US); Pengju Li, Chicago, IL (US); Wen Li, Chicago, IL (US)

(21) Appl. No.: 18/466,204

(22) Filed: Sep. 13, 2023

Related U.S. Application Data
(60) Provisional application No. 63/407,385, filed on Sep. 16, 2022.

(51) Int. Cl. C12N 13/00; C12M 1/42

(52) U.S. Cl. CPC C12N 13/00; C12M 35/02

(57) ABSTRACT

The present disclosure provides a device and method for a scalable production of extracellular vesicles (EVs) by electrical stimulation. Also provided herein is a composition comprising the EVs loaded with or without therapeutic agents and the method of using this composition for treating diseases.

### CROSS-REFERENCE TO RELATED APPLICATION

This application claims priority to U.S. provisional application No. 63/407,385, filed Sep. 16, 2022, the disclosure of which is expressly incorporated by reference herein.

### STATEMENT REGARDING FEDERALLY SPONSORED RESEARCH OR DEVELOPMENT

This invention was made with government support under W911NF-21-1-0090 awarded by the United States Army Research Office. The government has certain rights in the invention.

### FIELD OF THE DISCLOSURE

A device and method for the scalable bioelectrical production of extracellular vesicles are disclosed. The extracellular vesicles can be engineered for regenerative medicine, anti-tumor, and many other treatments.

### BACKGROUND OF THE DISCLOSURE

### (a) Extracellular Vesicles

Extracellular vesicles (EVs) are membranous particles secreted by nearly all types of cells. Based on their biogenesis and sizes, EVs can be divided into ectosomes (microvesicles, microparticles, or large vesicles in the size range of 100 nm to 1 μm in diameter)¹ that generated via outward budding from the plasma membrane and exosomes, the nanoscale vesicles (diameter: 30 to 200 nm) that generated with an endosomal origin² via the formation of intracellular multivesicular bodies (MVB), which are later released through MVB fusion to the plasma membrane (i.e., exocytosis).

Sequential invagination of the plasma membrane ultimately results in the formation of multivesicular bodies, which can intersect with other intracellular vesicles and organelles, contributing to diversity in the constituents of exosomes³. Depending on the biogenesis, exosomes can contain many components of a cell, including DNA, RNA, lipids, metabolites, cytosolic proteins, and membrane proteins. EVs with highly heterogeneous constituent are playing an essential role in intercellular communication, transporting biomolecules to regulate cellular processes, and supporting normal physiology²⁻⁴. The heterogeneity also makes EVs an ideal candidate for disease diagnostics⁴. For example, in neuroscience, most research focus on the roles that EVs play in neural disease mechanism and neuron-to-neuron/glial communication⁵⁻⁸. EVs mediate cellular communication in a feedback loop-like manner^{9,10}. Neurotransmitters stimulate oligodendrocytes to secrete vesicles, which are internalized by recipient neurons. Once internalized, the oligodendrocyte-derived cargo induces downstream neural responses, for instance, greater stress tolerance and increased viability. Therefore, neuron-derived EVs have been studied extensively to identify potential biomarkers for neuropsychological diseases, and analysis of the EVs profile and changes can benefit the studies of synaptic plasticity, neuronal stress response, and neurogenesis¹¹. Studies have suggested that exosomes may have a role in regulating intercellular communication based on the targeted accumulation of specific cellular components. Exosomes also ensure cellular homeostasis by removing excess and unnecessary cells components⁴ and influence the development of metabolic diseases as well as cardiovascular fitness. For example, exosomes and exosomal microRNAs promote cardiovascular health, possibly by promoting mitochondrial function, limiting cardiomyocyte apoptosis, and maintaining cardiac contractility¹²⁻¹⁴.

EVs are also promising drug-delivery vehicles possessing favorable pharmacokinetics and immunological properties for biomedical engineering¹⁵⁻¹⁷. EVs carry biomolecules and regulate many cellular processes by transporting biomolecules, such as proteins and genetic materials. Meanwhile, compared to other synthetic drug-delivery vehicles, exosomes, the smallest class of EVs, can penetrate physiological barriers almost impermeably (e.g., the blood-brain barrier), making exosomes widely used for targeted, localized delivery of chemical drugs to treat neural dysfunction¹⁸. Particularly, EVs are considered to have potential as ribonucleic acid (RNA) carriers, especially for microRNA (miRNA) and small interfering RNA (siRNA), due to excellent biocompatibility, bioavailability, minimal immunogenicity, structural proximity with cellular components, and various loading techniques, resulting in significantly high efficiency and stability of RNAs¹⁹⁻²³.

### (2) Production of Extracellular Vesicles

To apply EVs as clinically relevant therapeutic tools, EVs should contain bioactive ingredients necessary for therapeutic action. In addition to the quality of the content, EVs should be mass-produced to reach enough quantity. Since the composition of natural EVs is limited, EVs are mainly be used as therapeutical carriers. However, until now, accumulating enough therapeutic EVs in vitro has proven difficult due to the limited number of EVs can be generated per cell²⁴. Currently, mass production of EVs involves isolating EVs from biological fluids, such as blood, urine, and saliva, which ends up collecting heterogeneous EVs from multiple cell origin²⁵. It is still challenging and of significant concerns to create EV carriers with constant characteristics and properties on a large scale for encoding biopharmaceutical cargos. Therefore, new strategies to produce sufficient EVs carriers from sole cell sources on a large scale are still required.

A few strategies have been explored to solve the mass production challenge for EVs-based in vivo application, including applying external stimulation (such as serum deprivation²⁶, inducing hypoxia²⁷⁻²⁸, cellular nanoporation²⁹, mechanical forces³⁰, and high frequency acoustic³¹ or electrical³² stimulation), adjusting cell culture microenvironments by introducing cytokines³³, growth factors³² or altering calcium levels^{29,31,34}, and modifying the EVs biogenesis machinery, for example the knockdown of Rab27a, 27b, 35^{35,36}, or inhibition of acid sphingomyelinase decreased EVs production³⁷ while the overexpression of cortactin (an actin cytoskeletal regulatory protein) promotes exosomal secretion without altering the EVs cargo content³⁸ and the application of a production booster can achieve 15 to 20-fold increase in EVs production³⁹.

### (3) Bioelectrical Production of EVs

External electrical fields regulate cellular activities, such as calcium ion flow, action potentials firing, and synaptic communication. However, while many bioelectronic and electrical biointerfaces have been extensively applied in biomodulation to induce controllable biosignals, the impact of electrical field stimulation on widespread EVs signaling remains undetermined. As such, profiling real-time changes of EVs in live cell cultures under electrical stimulation remains interesting. Besides, the next wave of refinements in our understanding of bioelectronic interactions is likely to come from data at the subcellular or nanoscale level. As electric field can induce calcium influx during modulations, in principle, the profiles of EVs—their content, size, and amount—will change under electrical signals as EVs release is highly dependent on calcium influx^{31,34,40,41}, and electrical stimulation can alter the calcium profile of cells. However, no previous research has revealed the real-time impact of bioelectric modulations on EVs biogenesis.

The recently reported cellular nanoporation method using a biochip made up of 500 nm nanochannels generated 50-fold EVs amount and simultaneously increased (10³-fold) mRNA loading compared with bulk electroporation and other EVs production techniques (i.e., hypoxia, starvation, and heat stress). For the electroporation methods, membrane poration plays an important role, and it requires high voltages (100-220V) across the nanochannels for inducing exosome releases²⁹. There is still a need for additional research to better understand the role of electrical signals in modulating the formation of EVs.

### SUMMARY OF THE DISCLOSURE

The inventors have used a device to produce EVs, the device comprises a substrate, two interdigitated electrodes disposed on the substrate; and a controller electronically coupled to the two interdigitated electrodes and configured to apply to the two interdigitated electrodes an electrical stimulation sufficient to induce generation of extracellular vesicles from cells disposed on the substrate without killing the cells.

In certain embodiments, each of the two interdigitated electrodes comprises a respective plurality of conductive extensions, wherein each of the conductive extensions has a width of between 13 μm and 17 μm and a length greater than 200 μm and is separated from a conductive extension of the opposite electrode by a distance of at least 10 μm.

In certain embodiments, the substrate comprises glass or silicon.

In certain embodiments, the device further comprises a chamber that is coupled to the substrate and that encloses a volume that is exposed to the two interdigitated electrodes. Additionally, the chamber comprises an inlet for inflow of culture media into the chamber and an outlet for outflow of spent culture media and extracellular vesicles generated by cells disposed within the chamber. The chamber is also composed of PDMS.

In certain embodiments, the electrical stimulation is a biphasic electrical stimulation. The biphasic electrical stimulation comprises repeating biphasic waveforms, wherein each biphasic waveform comprises alternating voltage pulses of a negative pulse and a positive pulse. Each pulse has a magnitude between 0.25 V and 1.9 V. The biphasic waveforms also have a frequency between 0.5 Hz and 10 Hz and the voltage pulse lasts for 0.05, 0.1, 0.25, 0.5, 0.75, or 1 seconds. The biphasic waveforms also occur continuously for a time ranging from 20 seconds, 1, 2, 4, 8, 10, 12, 18, 24, 36, or 48 hours.

In certain embodiments, the device further comprises an imager configured to microscopically image cells disposed on the substrate in contact with the two interdigitated electrodes.

In certain embodiments, the substrate is optically transparent, and wherein the imager is configured to microscopically image the cells through the substrate.

In certain embodiments, the cells release EVs in response to the electrical stimulation.

Another aspect of the disclosure is a method of producing EVs comprising: culturing a population of cells on electrodes; stimulating the cells with the electrodes to induce EVs release; and collecting the released EVs. The cells' plasma membrane remain intact in this method.

In certain embodiments, the electrical stimulation delivered to the cells is a biphasic electrical stimulation. The biphasic electrical stimulation comprises repeating biphasic waveforms, wherein each biphasic waveform comprises alternating voltage pulses of a negative pulse and a positive pulse. Each pulse has a magnitude between 0.25 V and 1.9 V. The biphasic waveforms also have a frequency between 0.5 Hz and 10 Hz and the voltage pulse lasts for 0.05, 0.1, 0.25, 0.5, 0.75, or 1 seconds. The biphasic waveforms can occur continuously for a time ranging from 20 seconds, 1, 2, 4, 8, 10, 12, 18, 24, 36, or 48 hours.

The method of producing EVs described above can be applied in cell lines or primary cells such as HeLa cells, or cardiac fibroblasts respectively.

In the method of producing EVs described above, the cells are cultured to between 50% and 70% confluence when the electrical stimulation starts.

In certain embodiments, the produced EVs have larger size and improved sphericity compared EVs produced without the electrical stimulation. In certain embodiments, the EVs are exosomes.

Another aspect of the disclosure is a population of EVs produced from the method described herein, wherein the EVs have larger size and improved sphericity compared to EVs produced without the electrical stimulation.

Another aspect of the disclosure is a composition comprising the produced EVs and a pharmaceutically acceptable buffer, excipient, or carrier. The EVs can also be loaded with one or more of therapeutic agents.

Another aspect of the disclosure is a method of treating a subject with a condition, comprising administering a therapeutic amount of the composition comprising the produced EVs or the produced EVs loaded with one or more of therapeutic agents that the subject is in need thereof.

In certain embodiments, the one or more therapeutic agents can be nucleic acids, proteins, drugs or a combination thereof. More specifically, the nucleic acids can be RNA, microRNA, small interfering RNA, or a combination thereof.

In certain embodiments, the condition is cancer, cardiovascular disease, neurodegenerative disease, liver disease, kidney disease, respiratory disease, or tissue injury. In certain embodiments, the composition is delivered by intravenous, intramuscular, or intramyocardial injection.

### DESCRIPTION OF THE DRAWINGS

The disclosure will be better understood and features, aspects, and advantages other than those set forth above will become apparent when consideration is given to the following detailed description thereof. Such detailed description refers to the following drawings.

FIG. 1 is a diagram showing the working mechanism of CD63-pHluorin. i) CD-63 pHluorin, the MVB-PM fusion reporter, is not fluorescent inside the multivesicular bodies, where has a neutral pH. ii) the MVB-PM fusion events (i.e., exosome release events) lead to a burst of pHluorin fluorescent intensity with the pH shift from the intra-vesicular region (pH=5.5) to the extracellular region (pH=7.4); iii) the pHluorin signal decays due to diffusion in extracellular region. The inserted plot shows a representative signal of pHluorin during an exosomal secretion event. One secretion could generate a group of exosomes.

FIG. 2A to FIG. 2C show the general experimental setup and design. FIG. 2A is a diagram showing the experimental design and setup. Cells were transfected with CD63-pHluorin, the MVB-PM fusion reporter, to monitor real-time exosomal secretion during electrical stimulation delivered by planar interdigitated gold electrodes. FIG. 2B is a picture of the electrical live cell stimulation device on #1.5 glass, which allows for simultaneous super-resolution imaging. Scale bar, 1 cm. FIG. 2C shows the representative low-voltage low-frequency electrical waveforms (biphasic 1V, 2 Hz) for bioelectrical stimulation. Voltages were the input signals between the two interdigitated comb-shaped gold electrodes.

FIG. 3 shows the device fabrication workflow on #1.5 glass.

FIG. 4A to FIG. 4B show the bioelectronic stimulation device. FIG. 4A shows the electrode design. Two kinds of comb-like electrodes are interdigitated placed. The upper panels are electrodes has branches to increase spatial heterogeneity, and the lower panels are a pair of interdigitated parallel electrodes. FIG. 4B is a zoom-in view of the device design (left) and COMSOL Multiphysics finite element analysis of the electrical potential between the gap of two interdigitated gold electrodes (right).

FIG. 5A to FIG. 5C show the electrochemical characterization. FIG. 5A shows CV profiles of the gold interdigitated stimulation device measured in PBS with or without cell culture. FIG. 5B shows the bode plot of the measured impedance with or without cell culture. FIG. 5C shows the Nyquist Diagram of the measured impedance with or without cell culture. The changes indicating that cultured cells formed tight interfaces with the device, changing the gold surface properties.

FIG. 6A and FIG. 6B show SEM of rat cardiac fibroblasts (RCFs)/device interfaces. Scale bar, 50 μm for right panels; 10 μm for zoom-in views with pseudocolor.

FIG. 7 shows the stimulation voltage optimization. Live/dead analysis after applied bioelectrical stimulations. Scale bar, 50 μm.

FIG. 8A to FIG. 8C show calcium signals in electrical-stimulated HeLa cells. FIG. 8A shows that during ±1V, 2 Hz electrical stimulation, HeLa calcium levels remain stable, indicating intact membrane. FIG. 8B shows that during ±2V, 2 Hz electrical stimulation, HeLa cells quickly demonstrated elevated calcium, indicating membrane leakage as HeLa is not excitable cells. Scale bar, 20 μm. FIG. 8C shows that calcium intensity (F/F0) during electrical stimulation was measured at five randomly picked cells. Statistical analysis was performed using unpaired two-tailed t-test, indicating ±2V, 2 Hz significantly elevated calcium in HeLa after 60 s stimulation. **P<0.01. Reduced calcium during ±1V, 2 Hz stimulation was caused by bleaching.

FIG. 9A to FIG. 9B shows in vitro electrical stimulation with ±2V, 2 Hz. FIG. 9A is a TIRF image showing the CD63-pHluorin transfected HeLa cells on a gold electrode edge before bioelectrical stimulation. FIG. 9B are TIRF images showing the same transfected HeLa cells on the gold electrode edge after ±2V, 2 Hz bioelectrical stimulation for 1 min. Unlike exosomal release events that usually happened in small regions, the large area of fluorescent burst indicated the membrane leakage, which led to pH change and induced the pHluorin fluorescent signal. Scale bar, 10 μm.

FIG. 10A to FIG. 10C show bioelectrical stimulation induced exosomal release. FIG. 10A is a montage of super-resolution TIRF images of a HeLa cell cultured across the interdigitated gold electrodes (on #1.5 glass substrate). No bioelectrical stimulation was performed. FIG. 10B shows that upon bioelectrical stimulation of 1V for 0.25 s and −1V for 0.25 s (i.e., ±1V, 2 Hz signals) for 50 s, the HeLa cell across the interdigitated gold electrodes showed boosted exosomal secretion. Scale bar, 10 μm. c. 3D intensity at 45 s during ±1V, 2 Hz bioelectrical stimulation. Each peak indicates an exosomal secretion event.

FIG. 11A to FIG. 11D show the bioelectrical stimulation-boosted exosomal release. FIG. 11A show super-resolution TIRF images before electrical stimulation. No exosomal release was observed in the live cell. Scale bar, 5 μm. FIG. 11B shows that during ±1V, 2 Hz low-voltage low-frequency electrical stimulation, 17 exosomal release events (5 of which are labelled in FIG. 11B) were observed in 50 s recording window in a live HeLa cell. White dash lines in FIG. 11A and FIG. 11B, gold stimulation electrode position; bottom panel in FIG. 11A and FIG. 11B, intensity projection along the blue dash line showing the CD63-pHluorin intensity profiles over a time-lapse for 50 s. Each bright trace in the projection indicates an exosome release event. Scale bar, 5 μm. FIG. 11C shows five representative intensity profiles (selected exosome release events from FIG. 11A with position shown by the index) showing individual bioelectrical stimulation-induced exosomal release event. FIG. 11D shows quantification of the exosomal release observed in electrical-stimulated (±1V, 2 Hz) HeLa cells indicates that low-voltage low-frequency electrical stimulation can amplify exosomal production in vitro. The statistical analysis was performed using paired two-tailed t-tests; ** for P<0.01.

FIG. 12A to FIG. 12C show actin and CD63 colocalization. FIG. 12A is live-cell imaging of the actin (white) and pHluorin-CD63 (temporal color) shows the trafficking of multivesicular endosomes. Actin may control the trafficking of multivesicular endosomes (MVEs). FIG. 12B is the orthogonal view (depth is the timescale) of the actin (red) and pHluorin-CD63 (black) in live HeLa cells shows the alignment of traveling multivesicular body with the actin filament. Scale bar, 10 μm. FIG. 12B is a confocal microscopic imaging showing the distribution of actin (AF647-phalloidin) and CD63 (AF555-antiCD63). Scale bar, 10 μm.

FIG. 13A to FIG. 13C show super-resolution dSTORM micrographs of isolated EVs (HeLa cell source). FIG. 13A show EVs standards obtained from HCT116 human colorectal cell line (2.1×10¹¹ per ml), provided by Oxford Nanoimaging Inc., serving as a positive control with validated tetraspanin expression. FIG. 13B shows EVs isolated from HeLa cells cultured without electrical stimulation (2-hour culture in DMEM medium with 10% exosome-depleted FBS). FIG. 13C shows EVs isolated from electrical-stimulated HeLa cells (±1V, 2 Hz, 2-hour stimulation in DMEM medium with 10% exosome-depleted FBS). Colors are tetraspanins CD9 (blue), CD81 (red), and CD63 (green) extracellular vesicles biomarkers. Scale bar, 100 nm.

FIG. 14A to FIG. 14C show TEM and size quantification of isolated RCFs EVs. FIG. 14A shows representative TEM images of isolated EVs from RCFs without bioelectrical stimulation. Cells were cultured for 8 hours in DMEM medium with 10% exosome-depleted FBS. Selected regions of interest (ROIs) for EVs size quantification were indicated with yellow outlines. FIG. 14B shows TEM images of isolated e-EVs from RCFs after 8-hour bioelectrical stimulation (±1V, 2 Hz) in DMEM medium with 10% exosome-depleted FBS at 37° C. Selected ROIs for e-EVs size quantification were indicated with yellow outlines. FIG. 14C show EVs Feret length (or maximum calliper diameters) and aspect ratios measured from the ROIs in FIG. 14A and FIG. 14B. Compared to the EVs from non-stimulated RCFs, e-EVs obtained from bioelectrical-stimulated RCFs showed significantly larger sizes and improved circularity (i.e., sphericity, aspect ratios closer to one). Experiments to obtain EVs and e-EVs samples from RCFs were operated at the same time with the same procedures to exclude run-to-run variations. The statistical analysis was performed using unpaired two-tailed t-tests; mean value±S.E.M with individual data points were showed in the bar chart. **** for P<0.0001.

FIG. 15A to FIG. 15D show TEM and size quantification of isolated HeLa cell EVs. FIG. 15A shows representative TEM images of isolated EVs from HeLa cells without bioelectrical stimulation. Cells were cultured 12 hours on silicon in DMEM medium with 10% exosome-depleted FBS. FIG. 15B shows representative TEM images of isolated EVs from HeLa cells after 12-hour bioelectrical stimulation (±1V, 2 Hz) in DMEM medium with 10% exosome-depleted FBS. FIG. 15C shows selected regions of interest (ROIs) for EVs size quantification. Left, isolated EVs from non-stimulated HeLa cells; Right, isolated e-EVs from bioelectrical-stimulated HeLa (±1V, 2 Hz, 12 hours). FIG. 15D shows the 2D areas, Feret's diameters (or max. calliper diameter), and aspect ratios of the EVs and e-EVs in the ROIs in c. Compared to the EVs from non-stimulated HeLa cells, e-EVs obtained from bioelectrical-stimulated HeLa cells showed significantly larger sizes and improved circularity (i.e., sphericity, aspect ratios closer to one). Experiments to obtain EVs and e-EVs samples from HeLa cells were operated in parallel at the same time with the same procedures to exclude run-to-run variations. The statistical analysis was performed using unpaired two-tailed t-tests; mean value±S.E.M with individual data points were showed in the bar chart. **** for P<0.0001, and ** for P<0.01.

FIG. 16A and FIG. 16B show DLS measurements of EVs and e-EVs. EVs samples were obtained from HeLa cells cultured for 12 hours with (e-EVs) or without (EVs) electrical stimulation. EVs and e-EVs samples preparation and DLS measurements (in PBS) were operated in parallel with the same procedures to exclude run-to-run variations. FIG. 16A is the DLS distribution plot (mean±SD, N=10) indicated that e-EVs was significantly larger than EVs. FIG. 16B is the weighted averages of the hydrodynamic radius in PBS solution indicated that e-EVs was significantly larger than EVs.

FIG. 17A to FIG. 17E show scalable E-EVs production using large silicon device. FIG. 17A shows HeLa cells seeded onto silicon control wafer or device wafer with the same density and the medium was changed to DMEM with 10% exosome depleted FBS after cell adhesion and HeLa were cultured for 12 hours on the substrate, after which the media were collected and EVs was isolated for protein quantification using BCA assay. No obvious difference on different substrates was observed. FIG. 17B shows that EVs protein amount was quantified using BCA assay after 2-, 4-, 6-, or 12-hour culture on silicon without electrical stimulation or on device with electrical stimulation. EVs in media were then isolated and quantified. Picture shows the 4-in wafer scale devices (left) and silicon wafer control (right). FIG. 17C shows the fold change comparing electrical stimulation (ES) generated EVs amount with EVs amount in no ES culture on silicon. FIG. 17D is the standard curve and quantification of H₂O₂, an indicator of reactive oxygen species (ROS), shows minimal induced ROS after the 12-h electrical stimulation on device or 12-h culture on silicon. FIG. 17E shows that no significant production was observed in electrical stimulated (12-h) culture. Statistical analysis was performed using one way ANOVA tests. The bar chart shows individual data points and the mean value±S.E.M of multiple measurements. Ns for not significant or p≥0.05.

FIG. 18 is a photo of EVs pallet after filtering and high-speed centrifugation. Electrical stimulation for 8 hours generated more collected vesicles from the cell culture media compared to the non-stimulated control culture (cell source: HeLa cells).

FIG. 19A to FIG. 19B show cell viability analysis of RCFs. RCFs were cultured on device with and without electrical stimulation and on silicon substrate (as a non-stimulated no electrode negative control) for 12 hours. RCFs survival was promoted when stimulating with electrical stimulation compared to non-stimulated controls. FIG. 19A shows the flow cytometry analysis. FIG. 19B is the bar chart shows individual data points and the mean value±S.E.M of multiple measurements. Statistical analysis was performed using one way ANOVA tests with Tukey's multiple comparisons. * for P<0.05, ** for P<0.01, and **** for P<0.0001.

FIG. 20A to FIG. 20D show the bioreactor electrodes for exosome generation and continuous flow harvesting systems. FIG. 20A shows a bioreactor electrode fabricated on #1.5 glass slide for imaging. FIG. 20B shows silicon chips that can be fitted into continuous flow chambers. FIG. 20C shows the wafer-scale exosome generation electrodes. FIG. 20D is a photograph of the continuous flow systems showing the Arduino microcontroller boards, two peristatic pumps, transportation tubes and bioreactor chambers.

FIG. 21A to FIG. 21C show the engineering e-EVs with microRNA cargoes. FIG. 21A shows the isolated (e-EVs) loaded with miR199a-3p and miR210 microRNA mimics using Exo-Fect™ miRNA Transfection Kit, to further evaluate the therapeutic potential of using electrical stimulation produced EVs (e-EVs) as microRNAs carriers. FIG. 21B is a representative super-resolution image showing a miRNAs-e-EVs labelled with SYTO™ nucleic acid stain. Scale bar, 500 nm. FIG. 21C is a fluorescence imaging showing the recipient HeLa cells cultured with e-EVs that contained fluorescence-labelled siRNA cargo as positive loading control. Blue, nuclei; red, fluorescence-labelled siRNA. Scale bar, 20 μm.

FIG. 22A to FIG. 22E shows the flow cytometry cell viability analysis of CMs. CMs survival was promoted when culturing with miRNA-e-EVs. FIG. 22A and FIG. 22B show flow cytometric analysis of CMs cultured with and without miRNAs-e-EVs respectively. FIG. 22C and FIG. 22D show the quantification of calcein stain. FIG. 22E shows summary of CMs viability cultured with or without miRNAs-e-EVs. Statistical analysis was performed using unpaired two-tailed t-tests. The bar chart shows individual data points and the mean value±S.E.M of multiple measurements. *** for P<0.001.

FIG. 23 is a diagram summarizing the experimental design and timeline.

FIG. 24A to FIG. 24D show echocardiography evaluation of cardiac functions. FIG. 24A is a representative image showing the short axis view for M-mode. FIG. 24B is a representative image showing the parasternal long axis view for M-mode. FIG. 24C is a representative image showing the apical four-chamber view and E/A assessment under the Pulsed-Wave Doppler Mode. FIG. 24D is a representative image showing the apical four-chamber view and A′, E′ measurement under the Tissue Doppler Mode.

FIG. 25 shows mice heart rates during ECHO. Mouse heart rate was recorded during the long-axis M-mode and the short-axis M-mode ECHO assessment (N=3 for “No MI” mice, and N=5 for other experimental groups). Each bar chart shows the mean value±S.E.M and individual results. We recorded two heart rate values from each mouse, one from the long-axis M-mode and the other one from the short-axis M-mode. Two-way ANOVA with Dunnett's multiple comparisons tests were performed, comparing post-MI echo measurement with pre-surgery or Day 0 (pre-experiment) measured values. Not significant or p≥0.05 for all comparisons. The heart rate maintains stable during the experiment.

FIG. 26 shows representative electrocardiogram (ECG) recordings from mice on day 7 after MI surgery and treatment with materials. The PBS-treated mouse shows abnormal ECG patterns, while both e-EVs and miRNAs-e-EVs-treated mice shows similar patterns to the sham mice. Representative LV M-mode echocardiography images showing mice treated with e-EVs or miRNAs-e-EVs mostly recover from acute myocardial infarction.

FIG. 27A to FIG. 27B show ECG and spectrogram of a healthy mouse. The healthy mouse demonstrated stable ECG during the 30-day experiment. No surgery was performed on the mouse. FIG. 27A shows ECG signals measured at various experimental time points. FIG. 27B shows the corresponding ECG spectrograms. Red dash lines mark the pre-experiment ECG amplitude of the mouse.

FIG. 28A to FIG. 28B show ECG and spectrogram of a mouse with sham procedures. A control mouse with sham treatment procedures demonstrated stable ECG during the 30-day experiment. FIG. 28A shows ECG signals measured at various experimental time points. FIG. 28B shows the corresponding ECG spectrograms. Red dash lines mark the pre-surgery ECG amplitude of the mouse.

FIG. 29A to FIG. 29B show ECG and spectrogram of a MI mouse treated with PBS. The shape and amplitude of ECG were impaired after MI surgery and PBS (negative control) injection. After 30 days, the ECG was almost recovered. FIG. 29A shows ECG signals measured at various experimental time points. FIG. 29B shows the corresponding ECG spectrograms. Red dash lines mark the pre-surgery ECG amplitude of the mouse.

FIG. 30A to FIG. 30B show ECG and spectrogram of a MI mouse treated with e-EVs. A MI mouse with e-EVs treatment demonstrated recovered ECG. FIG. 30A shows ECG signals measured at various experimental time points. FIG. 30B is the corresponding ECG spectrograms. Red dash lines mark the pre-surgery ECG amplitude of the mouse.

FIG. 31A to FIG. 31B show ECG and spectrogram of a MI mouse treated with miRNAs-e-EVs. A MI mouse with miRNAs-e-EVs treatment demonstrated recovered ECG. FIG. 31A shows ECG signals measured at various experimental time points. FIG. 31B is the corresponding ECG spectrograms. Red dash lines mark the pre-surgery ECG amplitude of the mouse.

FIG. 32A to FIG. 32C show analysis of ECG Q-R-S complex. FIG. 32A is a representative ECG from a mouse without surgery shows the example of Q, R, S wave detection (left) and the example of rise level and fall level (right). FIG. 32B demonstrates that PBS-treated MI mice shows diminished rise level following PBS injection. FIG. 32C demonstrates that PBS-treated MI mice shows diminished fall level following PBS injection. ECG signals were measured on the same mouse at different time points (pre-surgery or pre-experiment, and 1, 3, 7, 30 days after MI surgery). Ten Q-R-S complexes were detected in each ECG signal. Rise levels measured from the Q-to-R in ECG signals and fall levels measured from the R-to-S in ECG signals were normalized according to pre-surgery or pre-experiment health mice condition. All statistical analysis was performed using ordinary two-way ANOVA tests with Dunnett's multiple comparison. Pre-surgery or pre-experiment values were used as normalization and comparison references. Each bar chart shows individual values obtained from QRS analysis (n=10). Error bar, SD. *P<0.05, **P<0.01, ***P<0.001, and ****P<0.0001. Not significant or p≥0.05 wherever not indicated.

FIG. 33A to FIG. 33F show signal similarity between pre- and post-MI ECG signals. FIG. 33A to FIG. 33E show cross-correlation (signal similarity) between pre- and post-MI ECG signals of a health mouse (FIG. 33A, no surgery was performed), a mouse with sham surgery procedures (FIG. 33B), a MI mouse treated with miRNAs-e-EVs (FIG. 33C), a MI mouse treated with miRNAs-e-EVs (FIG. 33D), and a MI mouse treated with PBS (FIG. 33E). FIG. 33F show box plot of the ECG signals similarity to the pre-surgery measurements. PBS demonstrated lowest similarity at day 1, day 3, and day 7 post-MI (marked by the yellow arrows), while miRNA-e-EVs shows the largest similarity in day 1, day 3, and day 7 post-MI surgery. Pre-surgery self-comparison was used for normalizing the correlation magnitude (amplitude set to 1.0).

FIG. 34 shows representative LV M-mode echocardiography images. Mice treated with e-EVs or miRNAs-e-EVs nearly recovered from acute myocardial infarction.

FIG. 35 shows ejection fraction evaluation. e-EVs or miRNAs-e-EVs treatment can recover cardiac function to pre-surgery level, while PBS-treated mice show significantly heart failure with reduced ejection fraction (HFrEF). N>6 measurements each group. Error bar, SEM. Two-way ANOVA with Dunnett's multiple comparison tests were performed, comparing post-MI echo measurements with pre-surgery measured values. ** for P<0.01, *** for P<0.001, and not significant or p≥0.05 for all comparisons not marked on the graph.

FIG. 36A to FIG. 36D show ECHO evaluation of cardiac function. Fractional shortening (FS, FIG. 36A), cardiac output (CO, FIG. 36B), stroke volume (FIG. 36C), and LV mass (FIG. 36D) were calculated from both the long-axis M-mode and the short-axis M-mode (N=3 for “No MI” mice, and N=5 for other experimental groups). Each bar chart shows the mean value±S.E.M and individual results. Two-way ANOVA with Dunnett's multiple comparisons tests were performed, comparing post-MI echo measurement with pre-surgery or Day 0 (pre-experiment) measured values. * for P<0.05, ** for P<0.01, *** for P<0.001, **** for P<0.0001, and not significant or p≥0.05 for all pairwise comparisons not marked on the graph.

FIG. 37A and FIG. 37B show ECHO evaluation of LV diastolic function. E/A ratio (FIG. 37A) and E/e′ (FIG. 37B) were calculated from the apical four-chamber view PW Doppler Mode and the Tissue Doppler Mode. Measurements were repeated on each mouse (N>4 mice each group) and the bar shows the mean value±S.E.M. Two-way ANOVA with Dunnett's multiple comparison tests were performed, comparing post-MI echo measurement with pre-surgery measured values. * for P<0.05, ** for P<0.01, *** for P<0.001, **** for P<0.0001, and not significant or p≥0.05 for all comparisons not marked on the graph.

FIG. 38 shows histology phenotypic characterization. Mice hearts were harvested 30 days after MI surgery and material treatments. PBS was used as a negative control. Representative Masson's trichrome (MTC) staining images of the infarcted heart cross-sections were shown with blue indicated the fibrosis area. Scale bar, 1 mm.

FIG. 39 shows quantified fibrotic area from phenotypic characterization of the mice hearts. MI mice treated with e-EVs or miRNAs-e-EVs showed diminished fibrosis and improved tissue healing compared to the PBS-treated control groups. Error bar, SEM. Statistical analysis was performed using one-way ANOVA with Tukey's multiple comparisons. * P<0.05, ** P<0.01, and ns or P≥0.05 for other comparisons that not indicated on the graph.

FIG. 40A to FIG. 40D show mice growth evaluation at the end of the experiments. After MI treatment with e-EVs, miRNAs-e-EVs, and PBS (negative control) for 30 days, we collected the body weight (FIG. 40A), heart weight (FIG. 40B), and tibia length (FIG. 40C). The growth was also normalized by obtaining heart weight/tibia length value (FIG. 40D). Mice treated with e-EVs or miRNAs-e-EVs (Cell source: RCFs, N=5 mice) grew similarly to the sham mice (N=5), without statistical significance. Mice without any surgery were used as healthy control (N=3). Error bar, SEM. Statistical analysis was performed using one-way ANOVA with Dunnett's multiple comparisons to the sham or “No MI” control group. ns or P≥0.05 for all pairwise comparisons and not indicated on the graph.

FIG. 41 is a boxplot of statistical p values in mice MI studies. Summarized statistical p values in mice MI model studies showed that e-EVs and miRNAs-e-EVs treatment repaired cardiac functions comparable to sham mice. Whiskers show minimum to maximum range with every individual data point. Statistical analysis was performed using one-way ANOVA with Tukey's multiple comparisons. * for P<0.05, *** for P<0.001, and ns or P≥0.05 in all other comparisons that not indicated on the graph.

FIG. 42 shows a heatmap of statistical p values in mice MI studies. The smaller the p-values or the larger the −log₁₀(p-value), the greater the statistical significance of the observed difference in each pairwise comparison. Specifically, PBS-treated mice showed the greatest statistical difference in mice MI studies. On the contrary, e-EVs and miRNAs-e-EVs treatment showed similar performance to the sham mice.

FIG. 43 shows a geometrical structure comprising of two interlocking comb-shaped arrays of finger electrodes.

### DETAILED DESCRIPTION

Before the disclosed methods and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the devices, methods, systems and compositions described herein can be configured by the person of ordinary skill in the art to meet the desired need. As described above, a method to induce EVs release from live cells by electrical stimulation and a device to produce the electrical stimulation are provided herein. Another aspect of the invention is a pharmaceutical composition comprising the EVs loaded with or without therapeutic agents and a method to treat a condition with the pharmaceutical composition.

Unless otherwise defined, all technical terms used herein have the same meaning as commonly understood by a person of ordinary skill in the art to which this disclosure belongs.

### 1. General Definitions

As used in the specification, articles “a” and “an” are used herein to refer to one or to more than one (i.e., at least one) of the grammatical object of the article.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### 2. Device for Inducing Electrical Stimulation-Generated Extracellular Vesicles (e-EVs)

### 2.1 the Electrodes

As provided herein, the electrodes used in the devices described above are planar interdigitated electrode arrays fabricated on silicon wafers, glass substrates, plastic substrates, or flexible material surfaces.

In certain embodiments, the electrodes are gold electrodes. Other materials made up the electrodes include, but are not limited to, platinum or palladium.

As used herein, the term “planar” refers to a common geometrical plane which the electrodes lay on. In certain embodiments, the planar electrode arrangement optimizes the contact between the cells and the electrodes, as the cells are cultured on the electrodes.

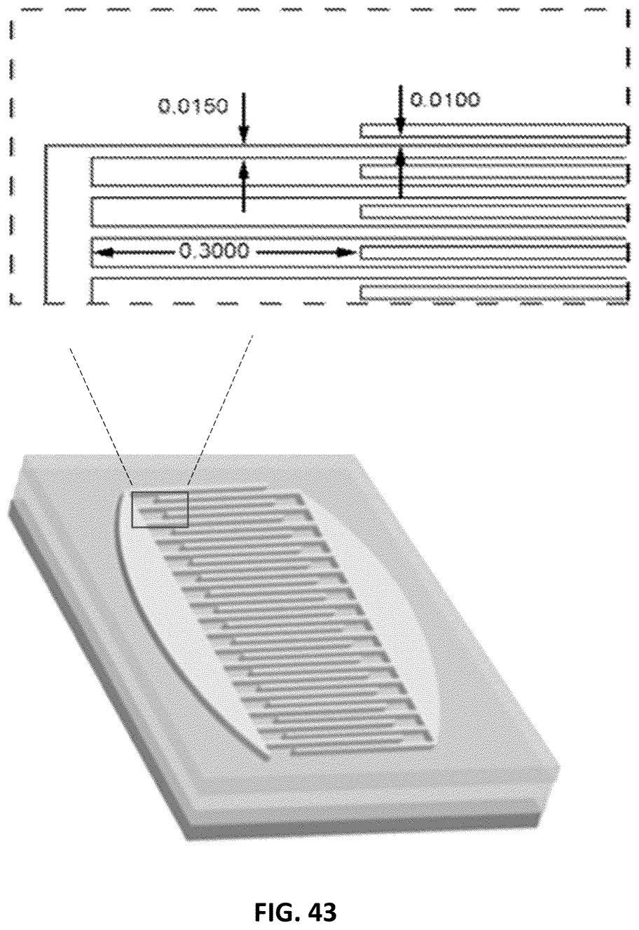

As used herein, the term “interdigitated” refers to a geometrical structure comprising of two interlocking comb-shaped arrays of finger electrodes, such as an example shown in FIG. 43. The electrodes are made up of two interdigitated electrodes, each comprises a respective plurality of conductive extensions (finger electrodes), wherein each of the conductive extensions has a width of between 13 μm and 17 μm and a length greater than 200 μm and is separated from a conductive extension of the opposite electrode by a distance of at least 10 μm. It is difficult to fabricate the electrodes if the separation is less than 10 μm.

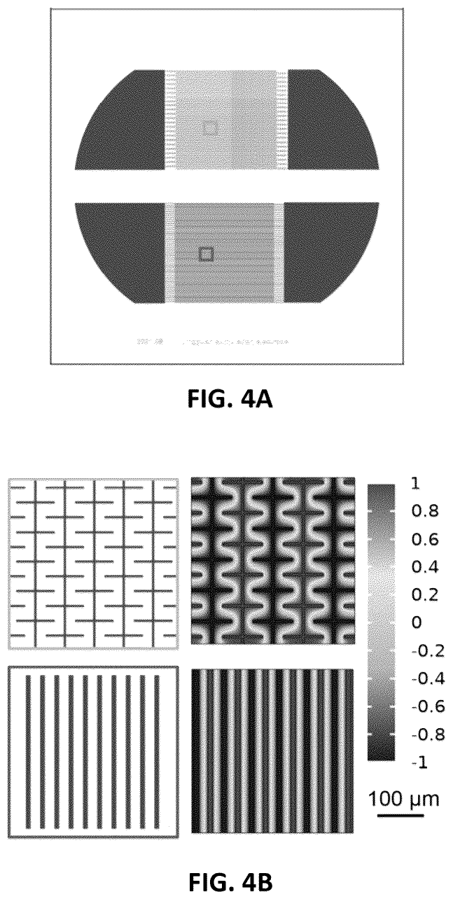

In certain embodiments, each conductive extension has a width of 15 μm, is separated from a conductive extension of the opposite electrode by a distance of 10 μm, and 300 μm distance to the edge of the comb, and are interdigitated parallel (FIG. 43 and FIG. 4B lower panel). In certain embodiments, the conductive extensions have branches to increase spatial heterogeneity (FIG. 4B upper panel). The single-channel device was composed of 150 pairs of electrodes connected to two large pads, allowing for manual connection to jumper wires.

The electrical stimulation from the interdigitated layout is advantageous as it can achieve single cell level modulation uniformly across the entire device area. The confined electrical potential around the finger electrodes may help improve the efficiency for stimulation (FIG. 4B). The interdigitated electrode design advantageously shrinks the feature size of the otherwise bulky electrode for subcellular interfaces.

In certain embodiments, interdigitated gold electrodes are used. In certain embodiments, these electrodes are fabricated using photolithography on either silicon wafers (for high-through put EVs production) or on glass substrates (for imaging). Planer interdigitated electrodes for extracellular electrical cellular stimulation can be prepared either on silicon substrates for high-throughput exosome production or on glass substrates for spontaneous super-resolution imaging.

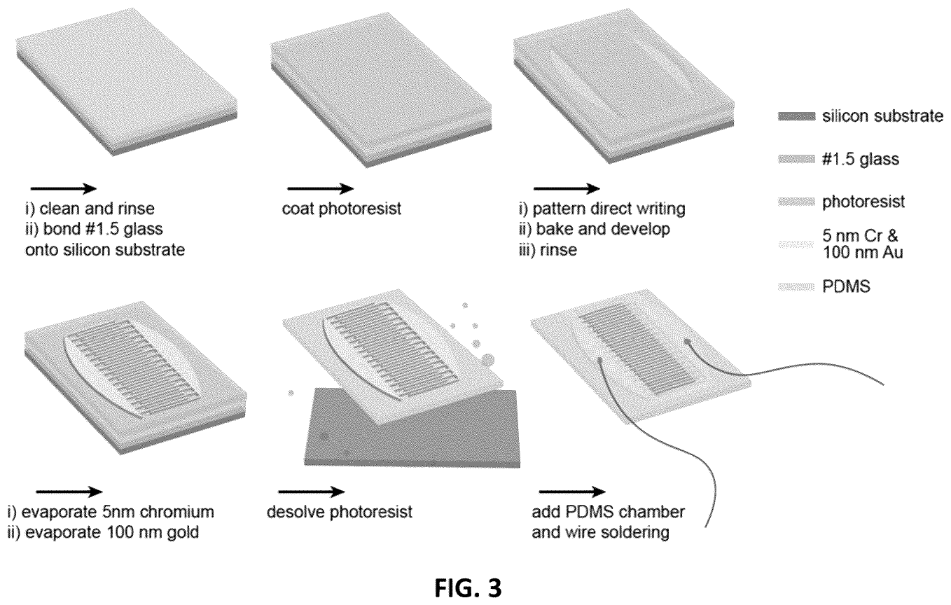

Interdigitated gold electrodes having a predetermined width and spacing can be fabricated using photolithography on commercially available glass substrates such as #1.5 glass slides (0.17-mm thickness) such as ClariTex coverglass for super mega slides, 64 mm×50 mm) available from Ted Pella Inc. For imaging applications, the glass substrate (or glass slide) can be cleaned, e.g., with acetone and isopropyl alcohol (IPA) in an ultrasonic bath (72 kHz), soaked in deionized (DI) water in sequential order, and dried using nitrogen gas. HMDS vapor priming can then be implemented on the glass substrate using a YES-58TA oven system to facilitate the wafer-to-photoresist adhesion. After vapor priming, the glass substrate can then be bonded onto a silicon carry wafer slide (55 mm×70 mm) utilizing a suitable photoresist such as AZ® 2070 photoresist to prevent thermal expansion of the glass during baking processes and undesirable detaching of cured photoresist on the glass due to uneven mechanical strains during baking. Afterwards, a AZ® 2020 photoresist can be spin-coated on the glass slide and soft baked at 110° C. for 2 min. After exposure using a Heidelberg direct writer (MLA150, 375 nm laser, 190 mJ/cm2, defocus +1), the photoresist was baked at 110° C. for 2 min and developed using AZ® 300MIF developer for 60 s. After DI water rinsing and 02 descum, 5 nm chromium and 100 nm gold can be evaporated on the patterned surface using an electron-beam evaporator (Angstrom EvoVac Electron Beam Evaporator). Finally, the photoresist can be stripped in remover PG at 80° C. for 6 h, during which the glass substrate bonded by AZ® 2070 also detached from the silicon wafer. In sequential order, the resulting patterned gold/glass slide may be cleaned using acetone, IPA, N2 gas, DI water, and N2 gas. A schematic of the fabrication process is shown in FIG. 3.

To form the cell-culture chamber for the electrodes, a mixture of 10:1 PDMS to curing agent can be molded and bonded to the electrode surface as follows. A standard mixture of 10:1 PDMS to curing agent can be prepared and poured onto a polyacrylic plastic mold cut by laser cutter and glued using a suitable solvent, e.g., dichloromethane. The PDMS in mode can be left to cure, e.g., for 2 h at 80° C. after degassing in a vacuum desiccator. Once the PDMS is fully cured, the PDMS layer can be peeled off. The device is cut with a blade and bonded onto the patterned glass after suitable treatment, e.g., oxygen plasma treatment (200 W, 2 min).

For large scale e-EVs production, 2D interdigitated gold electrodes can be fabricated on silicon wafers, e.g., 4-inch silicon wafers, instead of glass substrates using above mentioned photoresist, pre- and post-exposure baking, exposure, and development processing steps. On silicon wafers (or other materials) with larger surface area, a continuous flow system can be used to collect the e-EVs.

2.2 Small Scale Device for Use in Live Imaging of e-EVs Release

Devices that use the above electrodes (made with a glass or other optically transparent substrate) can be used for live imaging of the cells. This imaging can be performed while biphasic electrical stimulation is being applied to the cells via the electrodes on the substrate. Imaging can be performed of the cells while they are being electrically stimulated. This can include mounting the substrate with electrodes and cultured cells disposed thereon (e.g., in a chamber formed on the substrate) on or within a microscope or other imaging apparatus such that the cell-bearing surface of the substrate is located at an imaging focal plane of the imaging apparatus. In order to image the cells from beneath the substrate, the substrate and/or electrodes could be optically transparent. For example, the substrate could be composed of glass and/or the electrodes could be composed of transparent conductive material (e.g., indium tin oxide) and/or have a geometry (e.g., thickness) sufficient to permit the electrodes to be imaged through.

2.3 Large Scale Device for High Throughput Production of e-EVs

Devices that use the above electrodes (made with silicon substrate) and kept in a chamber with continuous flow system.

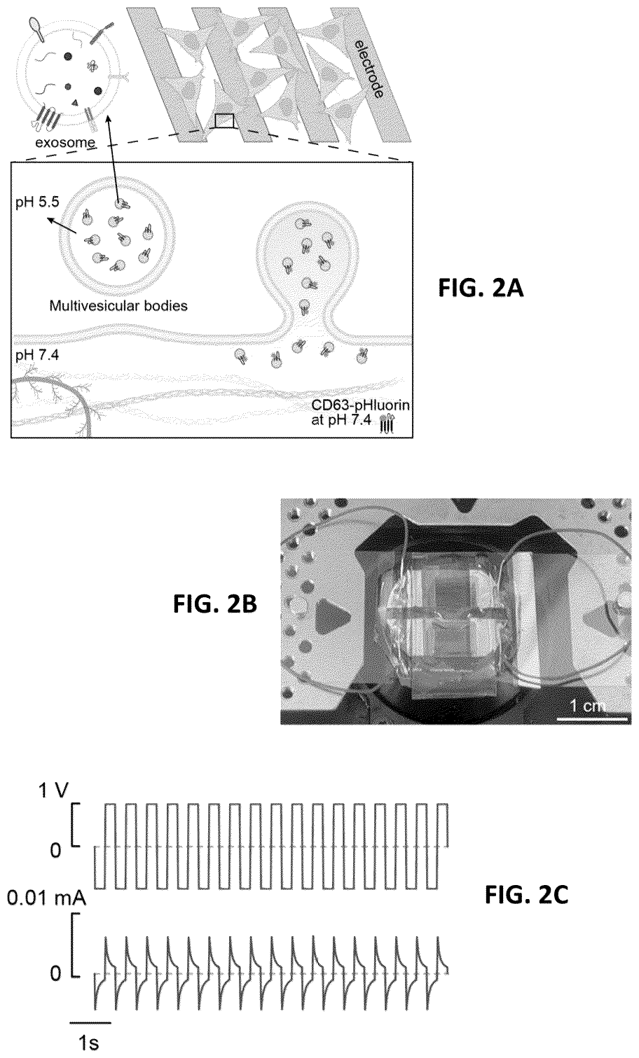

Special features of the device: 1) cells cultured on top of the electrodes (needs a culture well on top of the electrode) so in tight contact with the electrode (as opposed to other devices that just place two electrodes in the media containing the cultured cells 2) the stimulation is biphasic voltage input waveform (see FIG. 2C) as opposed to constant current input 3) plasma membrane of the cultured cells are intact (as opposed to making holes on the membrane).

Such a device could include a controller electrically coupled to the two (or more) interdigitated electrodes in order to apply biphasic electrical stimulus waveforms thereto that are sufficient to induce emission of extracellular vesicles while reducing or completely avoiding death of the cultured cells due to the application of the stimulus. This can include maintaining the magnitude of the biphasic voltage pulses applied to the electrodes below a specified level, e.g., below 2 volts applied from one electrode to the other. The magnitude of the applied voltage pulses could be selected to control an amount of vesicle release (e.g., to increase the amount of vesicle release) while avoiding or reducing cell death. For example, the magnitude of the applied voltage pulses could be between 0.25 volts and 1.9 volts. The frequency of the applied voltage pulses could also be controlled to increase vesicle release while reducing cytotoxicity. In certain embodiments, a biphasic electrical stimulation comprising repeating biphasic waveforms is applied. Each biphasic waveform comprises alternating voltage pulses of a negative pulse and a positive pulse, wherein each pulse has a magnitude at 0.25, 0.5, 0.75, 1, 1.25, 1.5, 1.75, or 1.9 V, preferably at 1 V. In certain embodiments, the biphasic waveforms have a frequency at 0.5, 1, 2, 3, 4, 5, 6, 7, 8, 9, or 10 Hz, preferably at 2 Hz. In certain embodiments, the voltage pulse lasts for 0.05, 0.1, 0.25, 0.5, 0.75, or 1 seconds, preferably at 0.25 seconds. In certain embodiments, the biphasic waveforms occur continuously for a time ranging from 20 seconds, 1, 2, 4, 8, 10, 12, 18, 24, 36, or 48 hours.

The applied biphasic waveform could be a controlled-current waveform and/or a controlled-voltage waveform. The controller used to generate the biphasic waveform and to apply it through the two electrodes could include controlled current sources, controlled voltage sources, potentiostats, amplifiers, clocks, switches, digital-to-analog converters, analog-to-digital converters, microcontrollers, buck, boost, or other voltage converter circuits, diodes or other over-voltage or over-current protection, and/or other elements to generate a specified biphasic waveform and to apply it to the two electrodes of a device as described herein in order to induce extracellular vesicle release by cells cultured on or near the electrodes while reducing or avoiding entirely the death of such cells due to the provided stimulus.

### 3. Method of Producing e-EVs

As provided herein is a method of producing e-EVs comprising culturing a population of cells on electrodes; stimulating the population of cells with electrical stimulation provided through electrodes; collecting e-EVs released from the population of cells, wherein the cells' membrane remain intact, and wherein the population of cells forms a tight interface with the electrodes.

As used herein, the term “electrical stimulation-generated extracellular vesicles” (e-EVs) refers to EVs that are released by cells after the cells are electrically stimulated. As demonstrated herein, this method is suitable to stimulate EVs release from stem cells, immortal cell lines (e.g. HeLa cells) or from primary cells isolated from living tissues or organs (e.g rat cardiac fibroblasts)⁶¹. Examples of immortal cell lines include, but are not limited to, HEK293, HeLa, HT1080, MC2F-7, K562, KB, HL-60, U-937, or Jurkat cells. Examples of primary cells include, but are not limited to, cardiac fibroblasts, hepatocytes, endothelial cells, neurons, cardiomyocytes, or epithelial cells. It has been contemplated that e-EV produced by this method can be applied on any type of cells capable of releasing EVs.

Methods of culturing the cells on the electrodes depend on the types of cells and are well known in the arts. The electrodes are fabricated with materials that are suitable for cell culture. Cells are cultured on the electrodes in media supplemented with ingredients such as nutrients or growth factors that are essential to sustain growth. In certain embodiments, HeLa cells are used and cultured in Dulbecco's Modified Eagle Medium supplemented with 10% Fetal Bovine Serum (FBS), 100 U/ml penicillin G, 100 mg/ml streptomycin sulfate, and 2 mM glutamine. In certain embodiments, primary rat cardiac fibroblasts are used and cultured in high glucose DMEM supplemented with 10% FBS, 1% GlutaMAX, and 1% penicillin-streptomycin. Most cultures are typically maintained at 37° C., 5% CO₂, and 100% humidity; unless they require special conditions.

Cells are typically seeded at between 20% to 30% confluence onto the electrodes and grown to between 50% to 70% confluence when the electrical stimulation starts. No more than 70% confluence should be achieved when the electrical stimulation starts. In certain embodiments, HeLa cells are seeded onto the electrodes at around 30% confluence (around 2×10⁴ cells/cm²) and grown within 1 day to reach 50% confluence for electrical stimulation. In certain embodiments, primary rat cardiac fibroblasts are seeded onto the electrodes at 30% confluence (2×10⁵ cells/cm²) and grown in 2 days to reach 50% confluence for electrical stimulation.

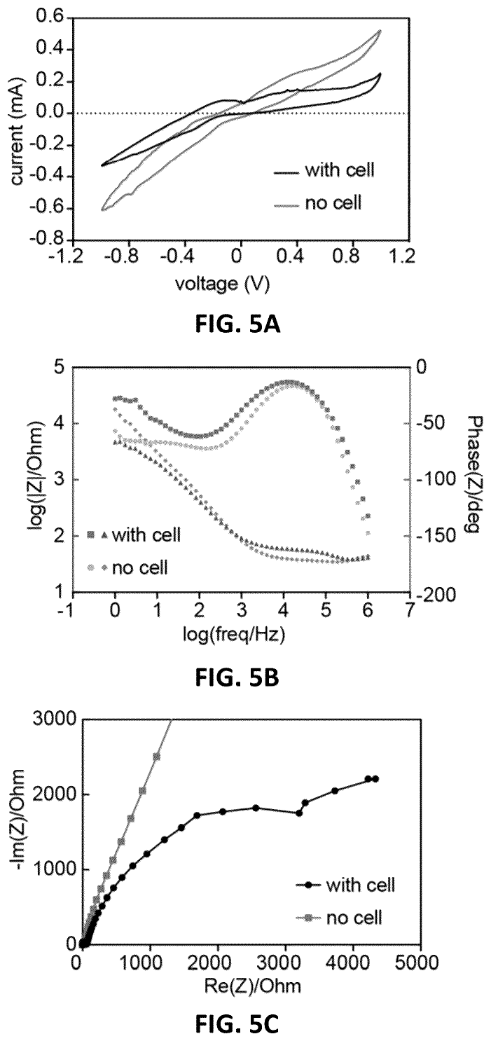

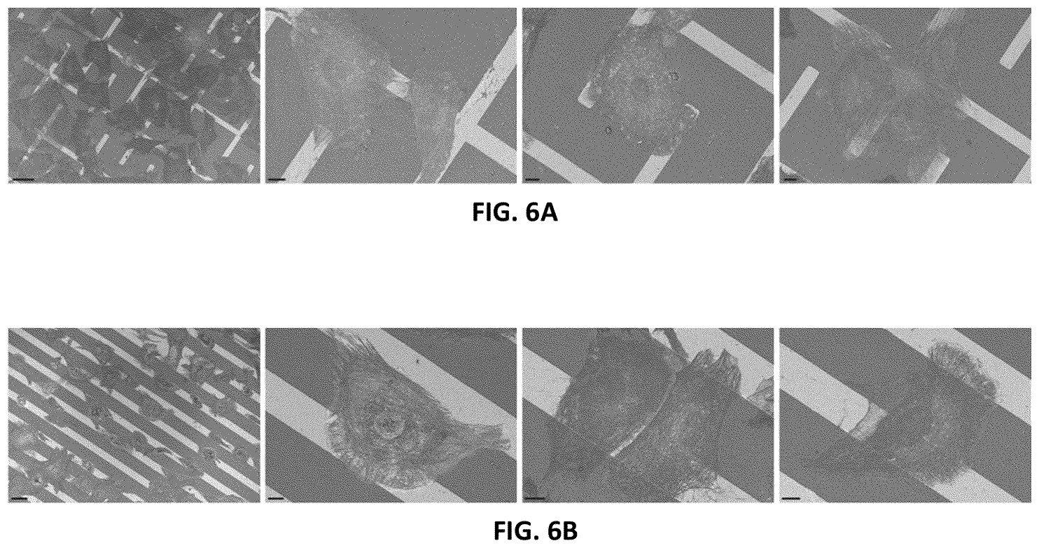

In certain embodiments, the electrodes are planar interdigitated electrode arrays. The planar interdigitated electrode arrays as described in section 2.1 can be incorporated in a device described in 2.2 or 2.3 to produce e-EVs. The cultured cells in this method are capable of forming tight interfaces on the electrodes as demonstrated in FIGS. 5A-5C and FIG. 6. This advantageously improves the efficiency of the electrical stimulation that the cells can experience. In addition, the electrical fields are better spatiotemporally controlled, because they can be defined by the shape of the electrodes (for example FIG. 2C and FIG. 4B) and distributed more uniformly across the cells, and can be adjusted temporally by the electrical waveform protocol.

The device can advantageously generate low-voltage low-frequency electrical waveforms that are sufficient to stimulate EVs release without killing the cells. In certain embodiments, a biphasic electrical stimulation comprises repeating biphasic waveforms is applied. Each biphasic waveform comprises alternating voltage pulses of a negative pulse and a positive pulse, wherein each pulse has a magnitude at 0.25, 0.5, 0.75, 1, 1.25, 1.5, 1.75, or 1.9 V, preferably at 1 V. In certain embodiments, the biphasic waveforms have a frequency at 0.5. 1, 2, 3, 4, 5, 6, 7, 8, 9, or 10 Hz, preferably at 2 Hz. In certain embodiments, the voltage pulse lasts for 0.05, 0.1, 0.25, 0.5, 0.75, or 1 seconds, preferably at 0.25 seconds. In certain embodiments, the biphasic waveforms occur continuously for a time ranging from 20 seconds, 1, 2, 4, 8, 10, 12, 18, 24, 36, or 48 hours depending on the cell type or the amount of desired EVs release. It should be noted that cells continue to grow during long duration of continuous electrical stimulation and may become overcrowded. It has been contemplated that overcrowded cells can reabsorb the released EVs. Thus, the cell confluence and the rate of cell growth should be considered when designing the duration of the electrical stimulation. In addition, EVs can be harvested every 1 to 2 hours to prevent reabsorption. The voltage, pulse duration, and protocol for electrical stimulation can also be customized for each cell type or the amount of desired EVs release. Other pulse-shapes such as triangle, gaussian or asymmetric have been contemplated to produce similar results as the biphasic protocol.

The EVs are released throughout the duration of the electrical stimulation. In certain embodiments, the media containing the released EVs is collected during or at the end of the stimulation. These EVs are called the collected EVs. The collected EVs can be isolated and purified using any methods commonly used in the arts⁶¹. In certain embodiments, the EVs are isolated by centrifugations and ultracentrifugations. Other techniques using tangential flow filtration or super absorbent polymer to collect the EVs, and chromatography for further purification have also been described elsewhere⁶¹ and contemplated herein.

In certain embodiments, the steps of electrically stimulating EVs and collecting the released EVs are performed at 37° C. to maintain the physiological temperature.

As used herein, the term “extracellular vesicles” (“EVs”) refers to small, enclosed, lipid bilayer membrane bound structures. EVs comprise exosomes and microvesicles which are originated from the endosomal system or the plasma membrane, respectively. The Examples in this disclosure demonstrate that electrical stimulation can induce exosome release from live cells. It has been contemplated that the same method would also be effective in stimulating microvesicles production.

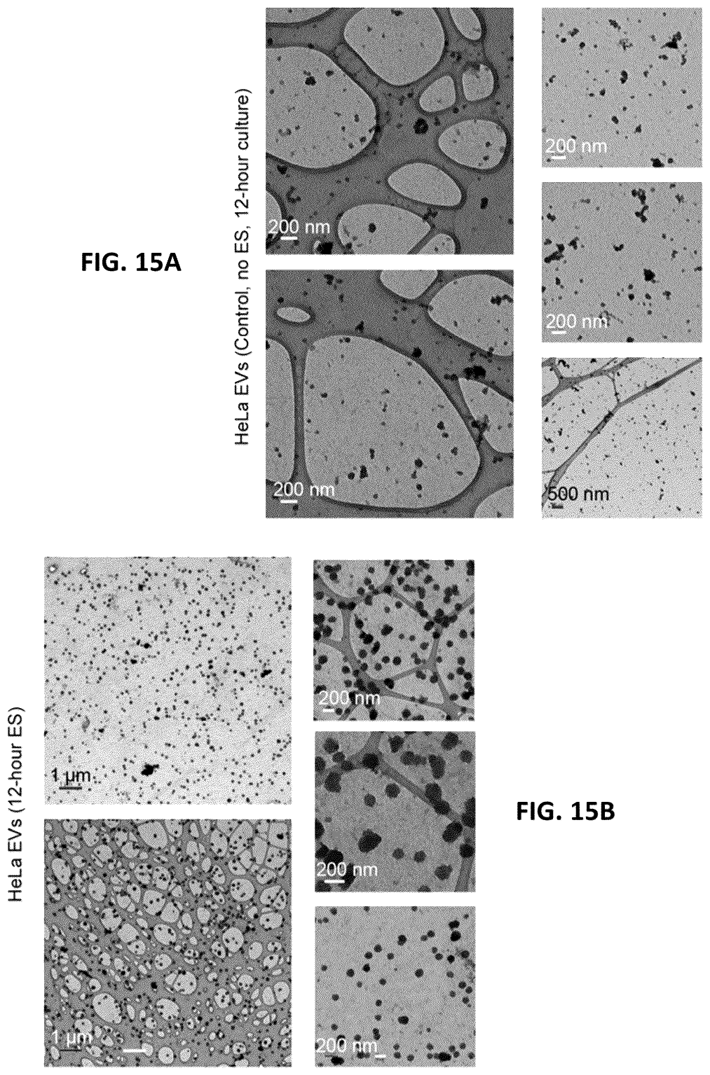

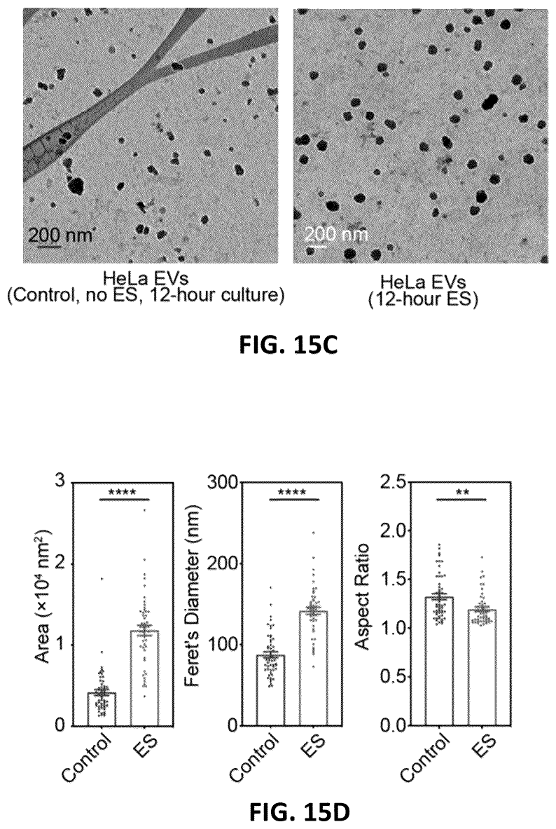

Based on their biogenesis and sizes, EVs can be divided into ectosomes (microvesicles, microparticles, or large vesicles in the size range of 100 nm to 1 μm in diameter)¹ that generated via outward budding from the plasma membrane and exosomes, the nanoscale vesicles (diameter: 30 to 200 nm) that generated with an endosomal origin² via the formation of intracellular multivesicular bodies (MVB), which are later released through MVB fusion to the plasma membrane (i.e., exocytosis). It is also found that the e-EVs produced by the described method show significantly larger sizes and improved sphericity compared to the EVs release without electrical stimulation (control). In certain embodiments, the EVs released by HeLa cells in response to the electrical stimulation are 1.5× larger in diameter, 3× larger in area compared to EVs released without electrical stimulation (FIG. 15D). This increase in EVs' loading capacity is particularly relevant for the application of e-EVs as delivery vehicles of therapeutic agents. In certain embodiments, the amount of EVs produced by electrical stimulation as described herein is 1.2, 1.5, 2, 2.5, 3, 4 folds of the amount of EVs produced without electrical stimulation. In certain embodiments, the protein concentration of the collected e-EVs is about 1.5 mg/mL in HeLa cells (10 mL can be collected) after 12 hours of electrical stimulation, corresponding to around 1.5× folds of the protein amount of EVs produced without electrical stimulation.

The collected e-EVs can be quantified by measuring the protein concentration using commercial kits such as the BCA protein assay kits. The number of e-EVs can be extrapolated by comparing the e-EVs's protein concentration with the protein concentration of the commercial EVs with known numbers of EVs. Alternatively, the number of collected e-EVs can be estimated by nanoparticle tracking analysis (NTA) from ONI nanoimager microscope⁶⁶.

It has also been contemplated that the described method to produce e-EVs can also be applied to electrically stimulate tissues ex vivo to induce EVs release. Examples of tissues include, but are not limited to, brain slices, blood vessels, and cardiac tissues. The tissues can be placed on the electrodes to maximize contacts. The tissue samples can be prepared on the same day and submerged in media that mimics the extracellular fluid. In some cases, the media can be supplied with oxygen to capture the native environment in the body. The strength of the voltage and duration might be optimized to minimize cell death and maximize EVs release.

Advantageously, the method to induce EVs release by electrical stimulation provided in this disclosure does not cause poration on the cells' plasma membrane. The recently reported cellular nanoporation method uses high voltages (100-220V) across the nanochannels to create membrane pores. These pores act as entering pathways for transcriptional factors that upregulate exosome releases²⁹. The method described herein uses low voltages with minimal impact on the cell integrity and introduces minimal external factors to induce EVs release. In another report, Fukuta et al. uses a constant current of 0.34 mA/cm² for 60 minutes to stimulate EVs release⁶³. Fukuta et al. also uses two Ag—AgCl electrodes placed on two ends of the culture plate on which the cells are seeded on. Thus, the cells are not in direct contact with the electrodes, and it is more challenging to control the electrical stimulation spatiotemporally. The method described here offers several additional advantages. Firstly, by employing closely spaced interdigitated electrodes, not only is a larger electroactive surface area achieved, but the distance for ion diffusion from the electrode to the cells is minimized. This leads to reduced interfacial resistance and fast charge transfer rates from electrodes to cells. Secondly, interdigitated electrodes exhibit a well-defined and controlled electrode-electrolyte interface, resulting in improved electrochemical performance. This translates into greater stability and predictability during charge and discharge cycles. Thirdly, the methodology outlined in this disclosure can be readily scaled up, which is crucial for the mass production of electric vehicles (EVs) on a large scale.

### 4. Pharmaceutical Formulations and Methods of Treatment

As provided herein is a method of treating a subject with a condition in vivo, comprising administering to a subject a composition comprising e-EVs produced using the device describe above, or the e-EVs loaded with therapeutic agents in a pharmaceutically acceptable carrier, wherein the pharmaceutical composition is administered in a therapeutically effective amount. Administration of the composition to a human subject or an animal in need thereof can be by any means known in the art for administering compounds. The condition can be cancer, cardiovascular disease, neurodegenerative disease, liver disease, kidney disease, respiratory disease, tissue injury and others.

It has been shown that EVs per se carry a plethora of molecules that play a role in several biological processes such as intercellular communication, transporting biomolecules to regulate cellular processes, and supporting normal physiology²⁴. In certain embodiments, e-EVs produced by the method described above are capable of restoring cardiac functions in myocardial infarction.

As used herein, “treatment” refers to the clinical intervention made in response to a disease, disorder, or physiological condition of the subject or to which a subject can be susceptible. The aim of treatment includes the alleviation or prevention of symptoms, slowing or stopping the progression or worsening of a disease, disorder, or condition and/or the remission of the disease, disorder, or condition.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The subject can be a human patient that is at risk for, or suffering from, a disease or a pathological condition. The human subject can be of any age (e.g., an infant, child, or adult).

The e-EVs described in this disclosure can be loaded with therapeutic agents. As used herein, the term “therapeutic agents” refers to any materials, biomolecules, or cells that can alleviate the symptoms in a disease or injury, or correct a disorder in a disease condition. Examples of therapeutic agents include, but are not limited to, nucleic acids, proteins, or drugs. In certain embodiments, the nucleic acids are RNA, microRNA, or small interfering RNA. Examples of drugs include, but are not limited to, anthocyanins, withaferin A, curcumin, paclitaxel, docetaxeI⁶⁴; and doxorubicin⁶⁵. In certain embodiments, the e-EVs can also be loaded with biological agents in general. The term “biological agent,” as used herein, refers to any compound or molecule that exerts a biological effect when delivered to a cell or a subject. This includes therapeutic, prophylactic, neutral, or toxic effects as well as detectable effects (e.g., a reporter molecule).

Methods of loading EVs with cargo are well known in the arts. In certain embodiments, EVs are incubated with therapeutic microRNA in transfection solution to facilitate uploading. Alternative methods such as sonication, extrusion, freeze and thaw cycles, electroporation, incubation with membrane permeabilizers, and others have been described elsewhere⁶².

The amount of the e-EVs or the loaded e-EVs used for therapeutic treatment depend on several factors, for examples, the method of delivery, the disease to be treated, and/or the amount of the cargos needed to reach the specific target. In certain embodiments, 40 μL e-EVs (concentration around 3.4×10¹⁰ of EVs per mL) solution is injected intramyocardially to treat myocardial infarction in mice. In certain embodiments, 200 μL e-EVs (concentration around 3.4×10¹⁰ of EVs per mL) loaded with 2 μL 100 μM miRNAs mixture (miR199a-3p and miR210 in 1:1 ratio) prior to its intramyocardial injection to treat myocardial infarction in mice.

The composition comprises the e-EVs or the loaded e-EVs with a pharmaceutical acceptable buffer, carrier or excipient can be formulated to suit the needs and routes of administrations. Non-limiting examples of formulations of the invention include those suitable for parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intramyocardial, intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site). In some embodiments, the formulation can be delivered locally to ischemic tissue. In certain embodiments, the formulation can be a slow release formulation, e.g., in the form of a slow release depot.

The particulars shown herein are by way of example and for purposes of illustrative discussion of certain embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed methods, compositions and devices are described, it is to be understood that the aspects described herein are not limited to specific embodiments, apparatus, or configurations, and as such can, of course, vary. It is also to be understood that the terminology used herein is for the purpose of describing particular aspects only and, unless specifically defined herein, is not intended to be limiting.

Embodiments of the present disclosure may thus relate to one of the enumerated example embodiments (Embodiment 1 to Embodiment 40) listed below:
Embodiment 1. A device comprising:
   a substrate;
   two interdigitated electrodes disposed on the substrate; and
   a controller electronically coupled to the two interdigitated electrodes and configured to apply to the two interdigitated electrodes an electrical stimulation sufficient to induce generation of extracellular vesicles from cells disposed on the substrate without killing the cells.
Embodiment 2. The device of embodiment 1, each of the two interdigitated electrodes comprises a respective plurality of conductive extensions, wherein each of the conductive extensions has a width of between 13 μm and 17 μm and a length greater than 200 μm and is separated from a conductive extension of the opposite electrode by a distance of between 8 μm and 12 μm.
Embodiment 3. The device of embodiment 1, wherein the substrate comprises glass or silicon.
Embodiment 4. The device of embodiment 1, wherein the device further comprises a chamber that is coupled to the substrate and that encloses a volume that is exposed to the two interdigitated electrodes.
Embodiment 5. The device of embodiment 4, wherein the chamber further comprises an inlet for inflow of culture media into the chamber and an outlet for outflow of spent culture media and extracellular vesicles generated by cells disposed within the chamber.
Embodiment 6. The device of embodiment 5, wherein the chamber is composed of PDMS.
Embodiment 7. The device of embodiment 1, wherein the electrical stimulation is a biphasic electrical stimulation.
Embodiment 8. The device of embodiment 7, wherein the biphasic electrical stimulation comprises repeating biphasic waveforms, wherein each biphasic waveform comprises alternating voltage pulses of a negative pulse and a positive pulse, wherein each pulse has a magnitude between 0.25 V and 1.9 V.
Embodiment 9. The device of embodiment 8, wherein the biphasic waveforms have a frequency between 0.5 Hz and 10 Hz.
Embodiment 10. The device of embodiment 9, wherein the voltage pulse lasts for 0.05, 0.1, 0.25, 0.5, 0.75, or 1 seconds.
Embodiment 11. The device of embodiment 10, wherein the biphasic waveforms occur continuously for a time ranging from 20 seconds, 1, 2, 4, 8, 10, 12, 18, 24, 36, or 48 hours.
Embodiment 12. The device of embodiment 1, further comprising:
   an imager configured to microscopically image cells disposed on the substrate in contact with the two interdigitated electrodes.
Embodiment 13. The device of embodiment 12, wherein the substrate is optically transparent, and wherein the imager is configured to microscopically image the cells through the substrate.
Embodiment 14. The device of embodiment 1, wherein the cells release EVs in response to the electrical stimulation.
Embodiment 15. A method of producing extracellular vesicles (EVs) comprising:
   culturing a population of cells on electrodes;
   stimulating the cells with the electrodes to induce EVs release; and
   collecting the released EVs, wherein the cells' plasma membrane remains intact.
Embodiment 16. The method of embodiment 15, wherein the electrodes comprise interdigitated electrodes.
Embodiment 17. The method of embodiment 16, wherein the electrical stimulation delivered to the population of cells is a biphasic electrical stimulation.
Embodiment 18. The method of embodiment 17, wherein the biphasic electrical stimulation comprises repeating biphasic waveforms, wherein each biphasic waveform comprises alternating voltage pulses of a negative pulse and a positive pulse, wherein each pulse has a magnitude between 0.25 V and 1.9 V.
Embodiment 19. The method of embodiment 18, wherein the biphasic waveforms have a frequency between 0.5 Hz and 10 Hz.
Embodiment 20. The method of embodiment 18, wherein the voltage pulse lasts for 0.05, 0.1, 0.25, 0.5, 0.75, or 1 seconds.
Embodiment 21. The method of embodiment 18, wherein the biphasic waveforms occur continuously for a time ranging from 20 seconds, 1, 2, 4, 8, 10, 12, 18, 24, 36, or 48 hours.
Embodiment 22. The method of embodiment 15, wherein the cells are stem cells, cell lines or primary cells.
Embodiment 23. The method of embodiment 15, wherein the cells are HeLa cells or primary cardiac fibroblasts.
Embodiment 24. The method of embodiment 15, wherein the cells are cultured to between 50% and 70% confluence when the electrical stimulation starts.
Embodiment 25. The method of embodiment 15, wherein the collected EVs have larger size and improved sphericity compared to EVs produced without the electrical stimulation.
Embodiment 26. The method of embodiment 15, wherein the EVs are exosomes.
Embodiment 27. A population of EVs produced from the method of embodiment 15, wherein the EVs have larger size and improved sphericity compared to EVs produced without the electrical stimulation.
Embodiment 28. A composition comprising the EVs produced in embodiment 15 and a pharmaceutically acceptable buffer, excipient, or carrier.
Embodiment 29. A composition of embodiment 28, wherein the EVs are loaded with one or more of therapeutic agents.
Embodiment 30. The composition of embodiment 29, wherein the therapeutic agents are nucleic acids, proteins, drugs, or a combination thereof.
Embodiment 31. The pharmaceutical composition of embodiment 30, wherein the nucleic acids are RNA, microRNA, small interfering RNA (siRNA), or a combination thereof.
Embodiment 32. A method of treating a subject with a condition, comprising administering a therapeutic amount of the composition of embodiment 28.
Embodiment 33. The method of embodiment 32, wherein the EVs are loaded with one or more of therapeutic agents.
Embodiment 34. The method of embodiment 33, wherein the subject is in need of the one or more of therapeutic agents.
Embodiment 35. The method of embodiment 34, wherein the one or more therapeutic agents are nucleic acids, proteins, drugs, or a combination thereof.
Embodiment 36. The method of embodiment 35, wherein the nucleic acids are RNA, microRNA, small interfering RNA (siRNA), or a combination thereof.
Embodiment 37. The method of embodiment 32, wherein the condition is cancer, cardiovascular disease, neurodegenerative disease, liver disease, kidney disease, respiratory disease, tissue injury.
Embodiment 38. The method of embodiment 32, wherein the composition is delivered by intravenous, intramuscular, or intramyocardial injection.
Embodiment 39. The method of embodiment 33, wherein the condition is cancer, cardiovascular disease, neurodegenerative disease, liver disease, kidney disease, respiratory disease, or tissue injury.
Embodiment 40. The method of embodiment 33, wherein the composition is delivered by intravenous, intramuscular, or intramyocardial injection.

Some embodiments of various aspects of the disclosure are described herein, including the best mode known to the inventors for carrying out the methods described herein. Of course, variations on these described embodiments will become apparent to those of ordinary skill in the art upon reading the foregoing description. The skilled artisan will employ such variations as appropriate, and as such the methods of the disclosure can be practiced otherwise than specifically described herein. Accordingly, the scope of the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### EXAMPLES

Certain aspects of the disclosure are illustrated further by the following examples, which are not to be construed as limiting the disclosure in scope or spirit to the specific methods and materials described in them.

### Example A: Experimental Procedures

### A.1 Device Fabrication

Device design was performed in AutoCAD software. Electrodes had a width of 10 μm with 40 μm spacing between the two interdigitated electrode comb. Interdigitated gold electrodes were fabricated using photolithography on #1.5 glass (0.17-mm thickness) substrates (Ted Pella Inc., ClariTex coverglass for super mega slides, 64 mm×50 mm) to allow for simultaneous super-resolution imaging. First, the thin glass was cleaned with acetone and IPA in the ultrasonic bath (72 kHz), soaked in DI water in sequential order, and dried using nitrogen gas. HMDS vapor priming was then implemented on the glass slide using the YES-58TA system to facilitate the adhesion. Then the glass substrate was bonded onto a silicon carry wafer slide (55 mm×70 mm) utilizing AZ2070 photoresist to prevent thermal expansion of glass during baking processes. This step is critical to avoid the detaching of cured photoresist on the glass due to uneven mechanical strains during baking. The photoresist was selected as an easily solvent-removable, clean-room accessible, and thickness-controllable adhesive. In contrast, conventional adhesives, such as Crystalbond™, are hard to apply and remove, and the thickness is hard to control. AZ2020 photoresist was spin-coated on the glass and soft baked at 110° C. for 2 min. After exposure using a Heidelberg direct writer (MLA150, 375 nm laser, 190 mJ/cm2, defocus +1), the photoresist was baked at 110° C. for 2 min and developed using AZ 300MIF for 60 s. After DI water rinsing and 02 descum, 5 nm chromium and 100 nm gold were evaporated on the patterned surface using an electron-beam evaporator (Angstrom EvoVac Electron Beam Evaporator). Lastly, the resist was stripped in remover PG at 80° C. for 6 h, during which the glass substrate bonded byAZ2070 also detached from the silicon wafer. In sequential order, the patterned gold@glass was cleaned using acetone, IPA, N2 gas, DI water, and N2 gas. A schematic of the fabrication process is shown in FIG. 2. For large scale e-EVs production, 2D interdigitated gold electrodes were fabricated on 4-inch silicon wafers instead of #1.5 glass using abovementioned photoresist, pre- and post-exposure baking, exposure, and development.

### A.2 Device Chamber and Wiring

A mixture of 10:1 PDMS to curing agent was poured onto a homemade polyacrylic plastic mold cut by laser cutter and glued using dichloromethane. The PDMS in mode was left to cure for 2 h at 80° C. after degassing in a vacuum desiccator. Once the PDMS is fully cured, the PDMS layer is peeled off. The device is cut with a blade and bonded onto the patterned glass after oxygen plasma treatment (200 W, 2 min).

A large pad connected to each comb-shape single-channel electrode allowed for manual connection to jumper wires. Jumper wires were connected to the pads via soldering, and the connections and excess connecting traces were insulated using silicone glue (Kwik-Sil, World Precision Instruments). Devices were sterilized with 70% ethanol and ultraviolet light and washed three times with PBS before the cell culture.

### A.3 Cell Culture

HeLa cells were cultured in high glucose Dulbecco's Modified Eagle Medium (DMEM, Corning™, 15017CV) supplemented with 10% Fetal Bovine Serum (FBS), 100 U/ml penicillin G, 100 mg/ml streptomycin sulfate, and 2 mM glutamine. Primary rat cardiac fibroblasts (RCFs) were isolated from hearts excised from neonatal rats (postnatal day 1-4) following the previous protocol. A Pierce™ primary cardiomyocyte isolation kit (Thermo Scientific, 88281) was used for digesting the tissue according to manufacturer protocol. RCFs were cultured in high glucose DMEM plus 10% FBS, 1% GlutaMAX, and 1% penicillin-streptomycin. All cultures were maintained at 37° C., 5% CO₂, and 100% humidity.

Before electrical stimulation and quantification experiments, the culture media was changed to DMEM media constructed with 10% exosome depleted FBS (System Biosciences LLC, EXOFBS50A1).

### A.4 CD63-pHluorin Transfection

The plasmid used for transfection was the pCMV-Sport6-CD63-pHluorin plasmid, a gift from DM Pegtel (Addgene #130901). The plasmid was amplified in Escherichia coli DH5a and was isolated and purified using the ZymoPURE DNA Plasmid Isolation Kit. DNA purity and integrity were determined spectroscopically. The DNA (100 ng/μL) was stored at −20° C. for future usage.

Plasmid transfections were performed using Fugene HD transfection reagent according to the manual on cells at 30-50% confluency. Fugene HD and pCMV-Sport6-CD63-pHluorin DNA (3:1) were added into Opti-MEM™ (Fisher scientific, 31985070) reduced serum medium and incubator at room temperature for 15 min before adding to cell culture. Cells were stimulated and imaged using TIRF after 48-72 h transfection.

### A.5 Bioelectrical Stimulation

Cells were seeded on the sterilized devices and cultured for 24 hours before electrical stimulation. Interdigitate electrode devices were connected to a potentiostat (SP-200, BioLogic), and biphasic voltage waveforms of various amplitudes of voltage, frequencies, and duration were delivered using the EC-Lab® Express (v.5.56) program. The electric potential was reported as the potential between the two individual interdigitated combs.

### A.6 COMSOL Simulation of Electrostatic Potential

The device structure constructed using AutoCAD was imported into COMSOL Multiphysics software (version 5.6) for performing finite element simulations of electrostatic potential between the interdigitated combs. The potential between the interdigitated electrodes was set to ±1 V.

### A.7 Electrochemistry Characterizations

Cyclic voltammetry (CV) and impedance measurements were evaluated with or without cell cultures on the devices using a potentiostat (SP-200, BioLogic) controlled by EC-Lab® Express (v.5.56). For the measurement, a pair of interdigitated gold electrodes on silicon substrate was connected as the working electrode, a platinum wire was connected as a counter electrode, and a Ag/AgCl (1 M) electrode was connected as a reference electrode. CV was performed within −1V to 1V voltage window with a scan rate of 100 mV/s, and potentiostatic electrochemical impedance spectroscopy (PEIS) measurements were performed from 1 Hz to 1000 kHz frequency range.

### A.8 Scanning Electron Microscope (SEM) Characterization of Cell-Device Interfaces

Afterwards, cells were then fixed in 4% paraformaldehyde (Electron Microscopy Sciences) and 0.4% glutaraldehyde (Sigma-Aldrich) in 0.2 M sodium cacodylate (Sigma-Aldrich) buffer (PH 7.2) overnight at 4° C. Fixed samples were washed using DI water and ethanol solution series (30%, 50%, 70%, 90%, 95%, and 100% ethanol sequentially) to reduce the water content. The samples were then dried using a critical point dryer (Leica EM CPD 300), during which carbon dioxide (304.13 K, 73.8 bar) was injected to exchange ethanol. Samples were then coated with 8 nm platinum-palladium (Pt/Pd) before SEM imaging to increase conductivity using a sputter coater (Ted Pella Cressington 208 HR). SEM images were taken at various magnification levels with an accelerating voltage of 2.0 kV.

### A.9 Real-Time Monitoring of Exosomal Secretion

The transfected cells expressing pHluorin-tagged CD63 were cultured on interdigitated gold electrodes on glass substrates to enable fluorescent imaging. The two individual combs were connected to electrical inputs for bioelectrical stimulations while simultaneous time-lapse Total Internal Reflection Fluorescence (TIRF) imaging. Imaging was performed using 488 nm laser setup (power<25% or <2.5 mW to minimize photobleaching) on the Oxford Nanoimager (ONI) with a 100× (1.4NA) oil immersion super apochromatic objective (Olympus) and an ORCA-Flash 4.0 V3 sCMOS camera (Hamamatsu Photonics). The ONI images field of view is 50 μm×80 μm and 0.117 μm pixel size. During all imaging and bioelectrical stimulation experiments, the temperature was controlled to 37° C., and Tyrode's solution (2 mM CaCl₂), 2.5 mM KCl, 119 mM NaCl, 2 mM MgCl₂, 30 mM glucose; pH 7.4) buffered with 25 mM HEPES was used as media in the stimulation PDMS chamber. Time-lapse images were captured at a frame rate of 10 frames per second (fps) before and during the electrical stimulation.

### A.10 Exosome Secretion Image Analysis

Time-dependent exosome secretion were analyzed at selected regions of interest (ROIs) with a burst fluorescent intensity. To see the exosomal production characteristic, the maximum intensity projection was obtained from the image stack using ImageJ, Z Project function. Bleach correction was conducted using exponential fit. 3D project was performed using imageJ function (color code: mpl-inferno; projection method: brightest point; axis of rotation: y-axis; initial angle: 90; total rotation and increment: 0). Fusion activity was defined as the number of events over a time-lapse experiment, which varied between experiments but was typically between 50 s to 100 s. N≥5 cells were imaged per condition in different imaging windows. Data shown are representative experiments replicated in independent experiments.

### A.11 Live-Dead Assay to Study Cell Viability

Cell survival after electrical stimulation was analyzed by flow cytometry and optical microscopy, respectively. Cells were stained with Hoechst 33342 (Thermo Scientific™, cat. 62249) to label cell nuclei s), propidium iodide (PI) to mark non-viable cells, and Calcein AM to identify live cells. Staining was performed for 15-20 minutes at room temperature in dark with a mixture of Hoechst 33342, PI, and Calcein AM of recommended dosages. For optical imaging, RCFs cultured and stimulation for 12 hours prior to staining. Imaging was performed using a Nikon eclipse Ti2 inverted microscope equipped with PCO.PANDA SCMOS camera. For flow cytometry, RCFs were cultured on large electrical stimulation device on 4-in silicon to obtain enough number of cells. After RCFs were stimulated for 12 hours, e-EVs were isolated from the supernatant, and RCFs were collected using trypsin treatment. Dissociated RCFs were washed with PBS and then stained with the abovementioned viability staining mixture. The viability of primary rat cardiomyocytes (CMs) was tested when culturing with miRNAs-e-EVs (EVs from RCFs cell source) for 12 hours using flow cytometry. Flow cytometry was performed on the BD LSRFortessa™ 4-15 HTS cell analyzer (BD Biosciences). Data were exported and analyzed using the Flowjo software (v. 10.7.1, BD).

### A.12 Calcium Imaging

For the Ca²⁺ experiments, live cells on device were labelled with Calbryte™ 520 AM calcium indicator (AAT Bioquest, 20650) according to in PBS buffer for 20-30 mins at 37° C. incubator with 5% CO₂. After incubation, cells were washed with PBS. The treated cells were then visualized with a Nikon eclipse Ti2 inverted microscope, using 60× oil immersion objective, N.A.=1.40. Time-lapse video of real-time calcium signaling in live cells were recorded with PCO.PANDA SCMOS camera during electrical stimulation.

### A.13 Device Recycle

To reuse the stimulation devices, 0.25% trypsin-0.02% EDTA (Sigma-Aldrich, 59428C), 10% sodium dodecyl sulfate (SDS) solution (Invitrogen™, 15553027), acetone, IPA, ethanol, UV, and PBS washing were sequentially applied for 30 min to clean and sterilize the gold electrode surface.

### A.14 Continuous Flow Exosome Harvesting Systems

An Arduino Uno microcontroller was connected with a four relays shield, which can be then used to control the on and off of the two peristaltic pumps. One pump is used to transport liquid from delivery tube to the black 3D printed culturing chamber through the small tubing design on the top right side. Another pump is used to transport liquid from culturing chamber to collection tube through the small tubing design on the bottom left side. The 3D printed black chamber is designed to contain multiple chips. The whole system is controlled with the Arduino IDE on the computer to achieve programmable media delivery, transportation, and collection.

### A.15 EVs Isolation

Cells were cultured in medium supplemented exosome depleted FBS. EVs were purified from cell culture supernatant after culturing with or without bioelectrical stimulation. The supernatant was centrifuged at 2000G for 10 min to remove cellular debris. The supernatant was transferred to a new tube, and total exosome isolation reagent (Invitrogen™, 4478359) was added 1:2 (v/v) to the cell-free culture media. After vertexing and storing under 4° C. overnight, the EVs were collected by centrifuging at 11200G for 1 hour. EVs are contained in the pellet at the bottom of the tube. After aspirating and discarding the supernatant, the EVs pellet was resuspended in 20-100 μl PBS for downstream analysis. The isolated EVs were kept at 4° C. for 1 week or at −20° C. for long-term storage.

A.16 EVs Super-Resolution dSTORM

dSTORM imaging was conducted to validate the presence of known EV membrane markers (e.g., the tetraspanins CD63, CD9, and CD81). Sample preparation was performed following the manufacturer's instructions using the EV Profiler Kit (Oxford Nanoimaging Inc.; product EV-MAN-1.0), which includes reagents for EVs capturing/immobilization, fixation, blocking, washing, dSTORM imaging buffers, and validated antibodies with dSTORM-compatible fluorophores for EVs labeling (i.e., anti-CD9-CF®488, anti-CD81-CF®647, and anti-CD63-CF®568 in 10 μg/ml working solution). Briefly, a 2.5 μl EV sample was added to a 3.5 μl blocking solution and mixed gently without introducing bubbles. After 5 mins incubation at room temperature, 3 μl of mixed antibodies (1 μl of each marker, ˜1 μg/ml final labeling concentration) was added to the EV solution, followed by gently mixing and incubating overnight at 4° C. The next day, after capture chip surface preparation, EV capture was performed immediately by incubating the antibody labeled EVs onto the capture chip for 15 mins at room temperature (which was done without exposing the chip to light). The chip lanes were washed with 100 μl wash buffer; then, 50 μl of fixation solution was added to each lane for 10 minutes and washed with 100 μl wash buffer. Next, dSTORM imaging buffer was added, then the chip lanes were immediately sealed with an adhesive strip to avoid liquid evaporation.

For three-color dSTORM acquisition, the Nanoimager S (Oxford Nanoimaging Inc.) camera was calibrated using 100 nm Tetraspek microspheres (Invitrogen T7279) diluted in PBS. Calibration beads were viewed under the Nanoimager using a 405/473/561/640 nm laser configuration with the 100× oil-objective lens. Images were taken using the NimOS software with 10 ms per frame and 5000 frames per channel. EVs from the HCT116 human colorectal cell line (2.1×1011 per ml), validated for tetraspanin expression, were used as a standard positive control.

The super-resolution dSTORM image was processed using the ONI image analysis cloud platform CODI (alto.codi.bio) for drift correction, filtering, and counting. The image frames were first filtered according to the laser sequence in which the localization was detected. Then the standard deviation of the fitted point spread (sigma) was set to 300 nm, and the localization precision was set to 10-30 nm. EVs were reconstructed by setting the length of the merged three-color clusters to be less than 2000 nm to filter out large aggregates. Representative CD9⁺, CD81⁺, and CD63⁺ triple-positive EVs images (pseudo-colored in blue for CD9, green for CD63, and red for CD81) were shown and analyzed for shapes, sizes, and marker distributions.

### A.17 Dynamic Light Scattering (DLS) Size Analysis

EVs or e-EVs were collected from RCFs after 12-hour culture with or without electrical stimulation. DLS experiments were performed using a DynaPro NanoStar DLS (Wyatt Technology Corp.), and data was analyzed using Dynamics version 7.8.1.3. software (Wyatt Technology Corp). DLS data were obtained at room temperature with more 5 duplicates of isolated EVs or e-EVs that were prepared in parallel to minimize run-to-run variations. According to the International standards organizations (ISO standards ISO 22,412:2017) that polydispersity index values<0.05 are more common to monodisperse samples. DLS data showed the size and monodispersity of EVs and e-EVs that were dissolved in PBS solutions.

### A.18 EVs Electron Microscopy Characterization and Size Quantification

Cells were cultured with or without electrical stimulation for 8 hours (RCFs) or 12 hours (HeLa cells) and EVs were isolated according to the abovementioned method. Isolated EVs or e-EVs were transferred to lacey carbon support film with ultrathin carbon film (Ted Pella. Inc., #01825) and fixed with 2% paraformaldehyde PBS solution and washed. Afterward, the adsorbed EVs or e-EVs were stained with uranyl acetate (2%) for 30 s, and samples were washed carefully with 100 μL distilled water for two times. After air drying, the EVs or e-EVs were imaged with a transmission electron microscope (TEM, FEI Tecnai G2 Spirit) under 120 kV. Experiments to obtain EVs and e-EVs samples from HeLa cells or RCFs were operated in parallel at the same time following same procedures to exclude run-to-run variations. Quantification of sizes (2D area, Feret's diameter, and EVs aspect ratios) was performed using ImageJ.

### A.19 E-EVs MicroRNA Loading

Therapeutics microRNA-199a (mmu-miR-199a-3p, Applied Biological Materials Inc., MCM01432) and microRNA-210 (mmu-miR-210, Applied Biological Materials Inc., MCM01489) were co-loading (1:1) into bioelectrical stimulation-generated EV carriers using Exo-Fect™ siRNA/miRNA transfection kit (System Biosciences, LLC, EXFT200A-1) according to manufacturer's instructions and recommended dosage: 10 μL Exo-Fect solution and 20 μL nucleic acid (20 pmol siRNA or miRNA) were added to 70 μL sterile 1×PBS, followed by adding 50 μL purified e-EVs PBS suspension (approximately 107 e-EVs). After gently mixed, the e-EVs transfection solution was incubated at 37° C. for 10 minutes on a shaker and moved onto ice before isolating the transfected e-EVs. Successful loading was verified by culturing non-fluorescent recipient HeLa cells with e-EVs loaded with fluorescence-labelled control siRNA and measuring the percentage of fluorescent cells.

### A.20 Animal

Wild-type C57B16 mice (6-8 weeks of age, male, weight around 20 g, n=5 for each experimental group) were obtained from Charles River Laboratories and housed in the animal facility of the University of Illinois at Chicago. The animal room was maintained at a humidity of 40-60% and a temperature of 18-23° C. under a 12-h-light/12-h-dark cycle. The animals were allowed free access to food and water. All the procedures were approved by the Ethics/Animal Care Committee of the University of Illinois at Chicago.

### A.21 Mice Acute Myocardial Infarction (AMI) Model

Prior to surgery, mice were weighed and given a subcutaneous injection of Buprenorphine SR LAB (1.0 mg/kg). Anesthesia was introduced using 1.5-3% isoflurane inhalation in a closed chamber. Aseptic procedures were used for all surgery and instruments were sterilized with a hot bead sterilizer between mice. Depth of anesthesia was monitored by assessing reluctance to move via lack of response to a toe-pinch. Surgical anesthesia was maintained using 2% isoflurane delivered through a vaporizer with air connected in series to rodent ventilator. The chest was shaved, and hair was removed via Nair® depilating agent. Skin was then scrubbed with betadine solution+70% isopropyl alcohol three times followed by sterile saline.

A left thoracotomy was performed via the fourth intercostal space to expose the heart. The left anterior descending coronary artery (LAD) was identified and temporally ligated. Ligation was released after 45 minutes, and the reperfusion phase started. Then e-EVs loaded with miR-199a-3p and miR-210 were suspended in 200 μl of PBS and delivered by four intramyocardial injections in four locations (10 μL each) to peri-infarct area within 5 minutes after reperfusion into each mouse. In this experimental setting, the delivery of the e-EVs directly following MI surgery avoids a second surgery, which would importantly increase animal mortality. PBS injection was used as a negative control treatment. The mice lung was hyperinflated by increasing positive end-expiratory pressure and the thoracotomy site was closed. The animals were observed and studied for four weeks post-surgery.

All these surgical procedures were performed in the University of Illinois at Chicago Cardiovascular Research Core. Phenotypic characterization for MI mice was conducted via echocardiography and histology.

### A.22 Echocardiography

Pre-surgery echo was conducted 24 hours before surgery, and on day 1 (24 hours after surgery), day 3, day 7, and day 30 using a 550 Hz transducer (VisualSonics Inc.) and the Vevo 2100 Imaging System (VisualSonics Inc.).

To prepare the mouse for echocardiography, a mouse was first anesthetized in a knock-down box using isoflurane (a concentration of 2-3%). The hair on the ventral side was removed using a depilatory cream (Nair®) and cleaned with Kimwipes® wipers before imaging. After applying a small amount of electrode gel (Parker Laboratories, Inc.) to the copper leads on the heated (37° C.) operation platform (VisualSonics, Inc.), the prepared mouse was positioned on the platform with ventral side up, and the paws were taped to the copper leads to provide the electrocardiogram (ECG). Respiratory waveform and respiratory rate were monitored simultaneously. All heart rates for mice were maintained over 400 bpm (beats per minute). A layer of pre-warmed ultrasound gel (Parker Laboratories, Inc.) was added onto the mouse chest before imaging.

To identify the anatomical structures and evaluate the heart systolic and diastolic function and myocardium health, parasternal long axis view (PLAX) and parasternal short axis view (PSAX) were collected using motion mode (M-mode) setting.

Regional cardiac functions were evaluated using M-mode images, where the left ventricular (LV) ejection fraction (EF), fraction shortening (FS), and cardiac output (CO) were calculated based on the following equations:

EF=(1−Systolic volume/Diastolic volume)×100%

FS=(1−Systolic internal diameter/Diastolic internal diameter)×100%

CO=(Diastolic volume−Systolic volume)×Heart rate/1000 (mL/min)

Echocardiographic data (LV mass, stroke volume, EF, FS, CO, E/A ratio, etc.) were analyzed and calculated using the Vevo LAB software (VisualSonics Inc., v.5.7.0). ECG spectrogram and similarity (correlation) between ECGs at various measurement time points was computed using MATLAB (R2020b, v9.9) and signal processing toolbox (v8.5). Spectrogram was set with normalized frequency (rad/sample) from 0 to 0.5×π. Similarity was normalized by setting correlation=1.0 when comparing pre-surgery measurement with itself.

### A.23 Tissue Harvest and Histological Analyses

At the end of the experiments, the mice were fully anesthetized, and the body weight, heart weight, and tibia length were measured 30 days after MI and sample injection. The excised hearts were collected, briefly washed in PBS, weighted, cut in half, fixed in 10% formalin at room temperature, embedded in paraffin, sliced into horizontal cross-section slices (5 μm section thickness; 200 μm step size), and further processed for histology. Masson's trichrome (MTC) staining were performed according to standard protocol and analyzed for morphology. The extent of cardiac fibrosis was measured on 20× magnification images and quantified using ImageJ by blinded researchers. Fibrosis was quantified by calculating the area of positive MTC staining region (blue) as a proportion of the total tissue cross-section area. The MTC staining region (blue) was selected using ImageJ color deconvolution function and threshold adjustment. For each experimental group, 3-5 mice were used.

### A.24 Statistical Analyses and Reproducibility

To analyze the statistically significant difference between the groups, unpaired two-tailed t-tests and ANOVA analyses were used. Data are represented as mean±SEM unless otherwise stated. Significance was claimed at *P<0.05, **P<0.01, ***P<0.001, and ****P<0.0001. The numbers of experimental replicates are given in the figure legends. An individual data point represents an independent bioelectrical stimulation experiment in all studies.

### A.25 General Data Processing

Analysis of numerical data was performed using Microsoft Excel. Statistical analyses were performed using GraphPad Prism 9.5 (GraphPad Software Inc). Plotting and graphical representation were performed using GraphPad Prism and Adobe Illustrator.

### Example 1: Bioelectrical Stimulation and Real-Time Observation of Exosomal Release

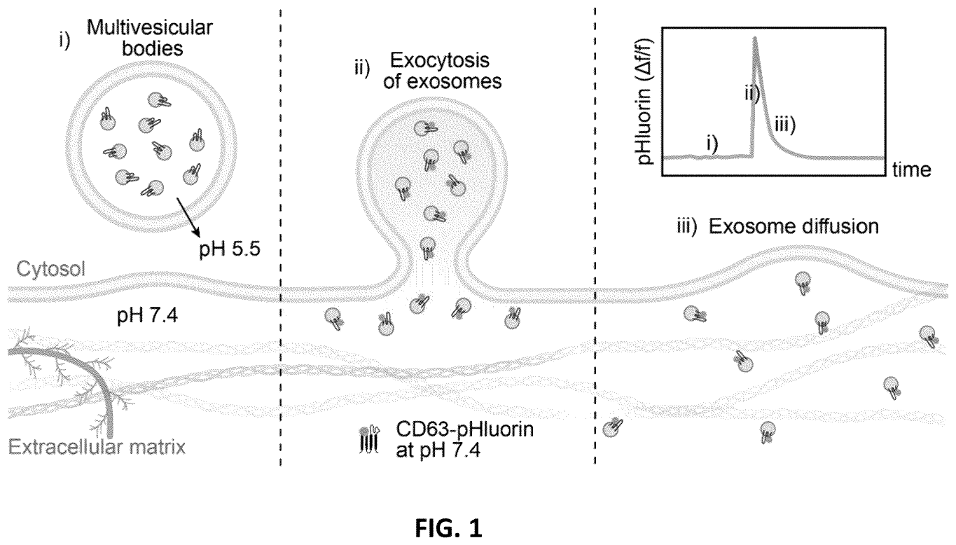

With the help of pHluorin-tagged CD63 exosome markers^{42,43}, the MVB-PM fusion events (i.e., exosome release events) can be visualized, appearing as a burst of fluorescent intensity generated from the pHluorin tag when experience a shift from the intra-vesicular region (pH=5.5) to the extracellular region (pH=7.4) as a corollary of the exosome release (FIG. 1). To evaluate the real-time impact of bioelectrical modulations on EVs biogenesis dynamics in live cells, transparent electronic devices were developed (FIG. 2A and FIG. 2B) to deliver spatially controlled electrical signals (FIG. 2C) and observed the cells super-resolution microscope upon electrical stimulation.

### Example 2: Preparation and Characterization of the Stimulation Device

A lithography was used to fabricate planer interdigitated electrodes for extracellular electrical cellular stimulation on glass slides (#1.5 or 0.17-mm thickness) so that spontaneous super-resolution imaging is applicable (FIG. 3). Interdigitated gold electrodes on silicon substrates were also fabricated according to traditional photolithography for non-imaging experiment. The devices were wired, and a well on the slide was fabricated using PDMS, where the cell can be cultured. The electric potential of stimulation devices was evaluated via finite element analysis using COMSOL Multiphysics simulations (FIG. 4A and FIG. 4B). Then the fabricated bioelectronics chip was integrated with the super-resolution imaging system for simultaneous bioelectrical modulation and imaging (FIG. 2) to evaluate the real-time impact of bioelectrical modulations on EVs biogenesis dynamics in live cells.

### Example 3: Bioelectrical Stimulations Amplified Exosomal Release

HeLa cells was used as an exploratory step to study the EVs dynamics in live cells under the spatiotemporally controlled electrical fields. HeLa cells were cultured and transfected with the pHluorin-tagged CD63. A burst of fluorescent intensity is generated from the pHluorin tag when CD63 experience a shift from the intra-vesicular region (pH=5.5) to the extracellular region (pH=7.4) as a corollary of the exosome release or MVB-PM fusion (FIG. 1). Therefore, it is possible to monitor subcellular exosomal releases and track MVB trafficking and fusion.

Transfected HeLa cells were cultured on the bioelectrical stimulation device on glass substrate, and cells formed tight interfaces on the gold electrodes, as shown by the altered cyclic voltammetry (CV) and impedance profiles (FIG. 5A-5C). Transfected primary rat cardiac fibroblasts (RCFs) were also cultured on device and scanning electron microscope (SEM) images verified the tight interfaces between cultured RCFs and the gold electrode on #1.5 imaging glass (FIG. 6A-6B). A desktop super-resolution microscope, the Oxford Nanolmager (ONI), was used to perform the total internal reflection fluorescence (TIRF) super-resolution time series recording during electrical stimulation.

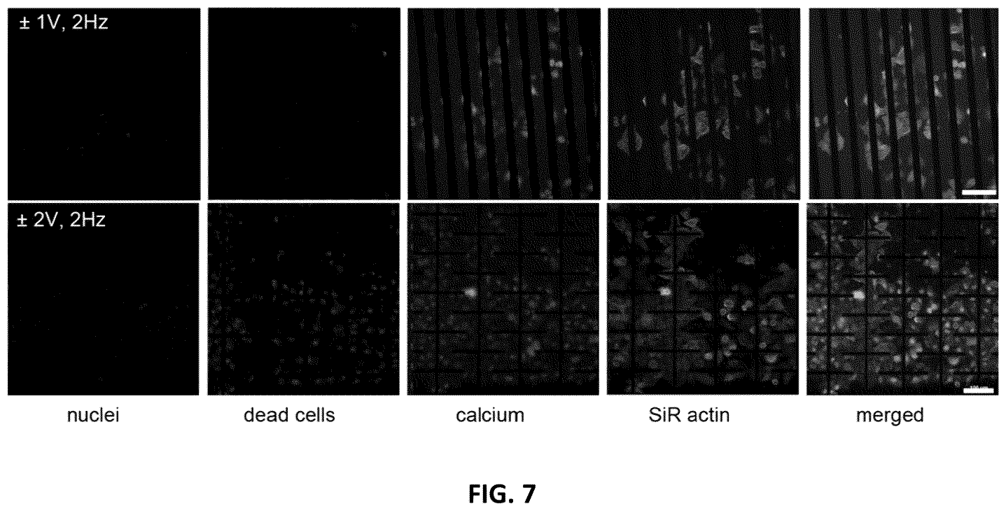

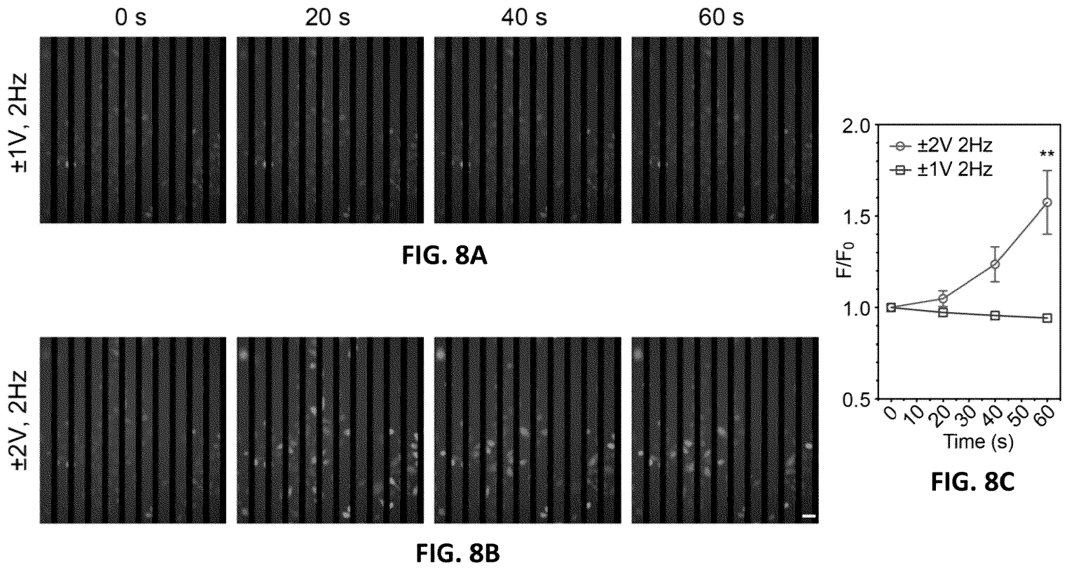

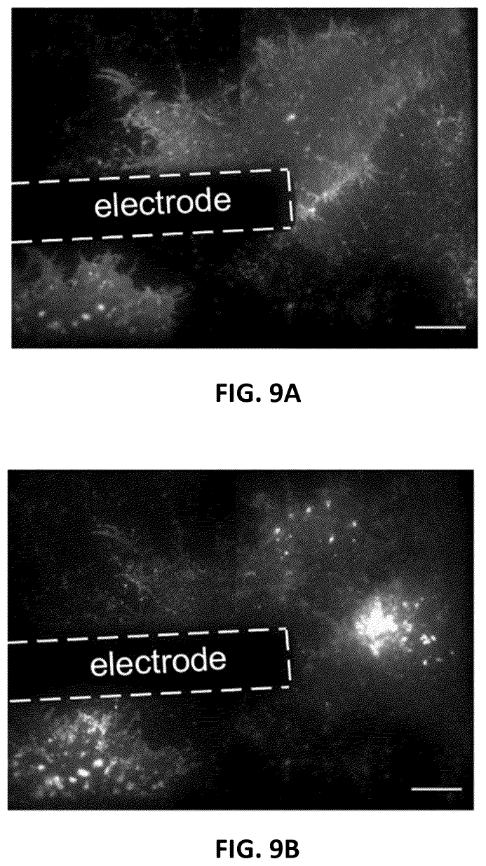

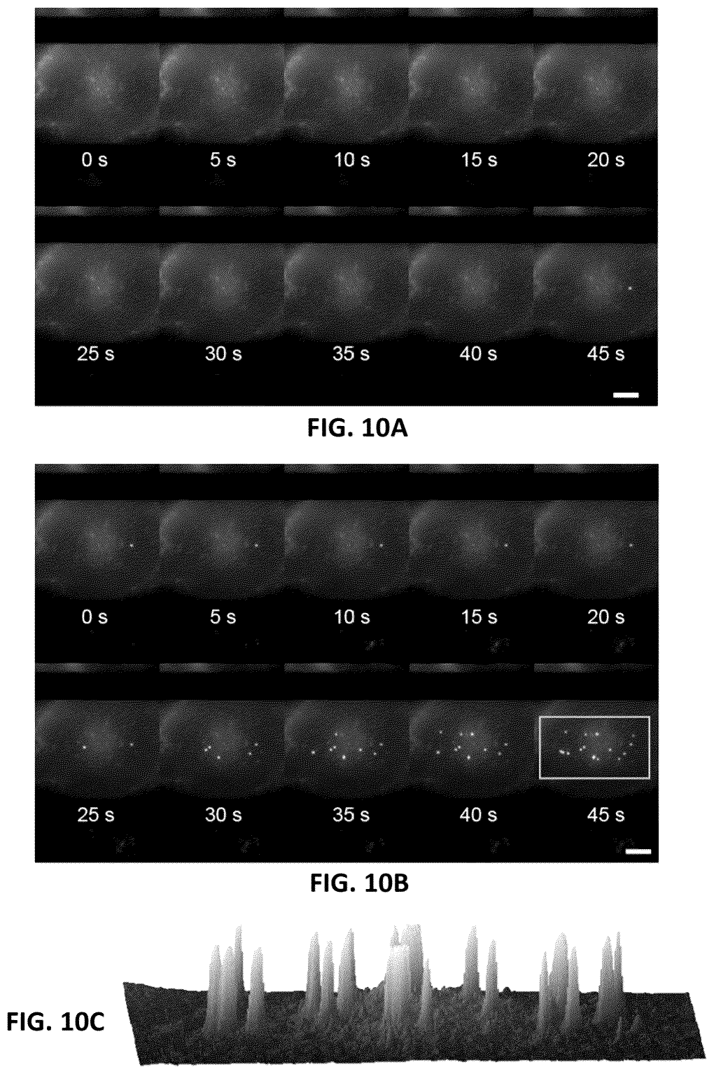

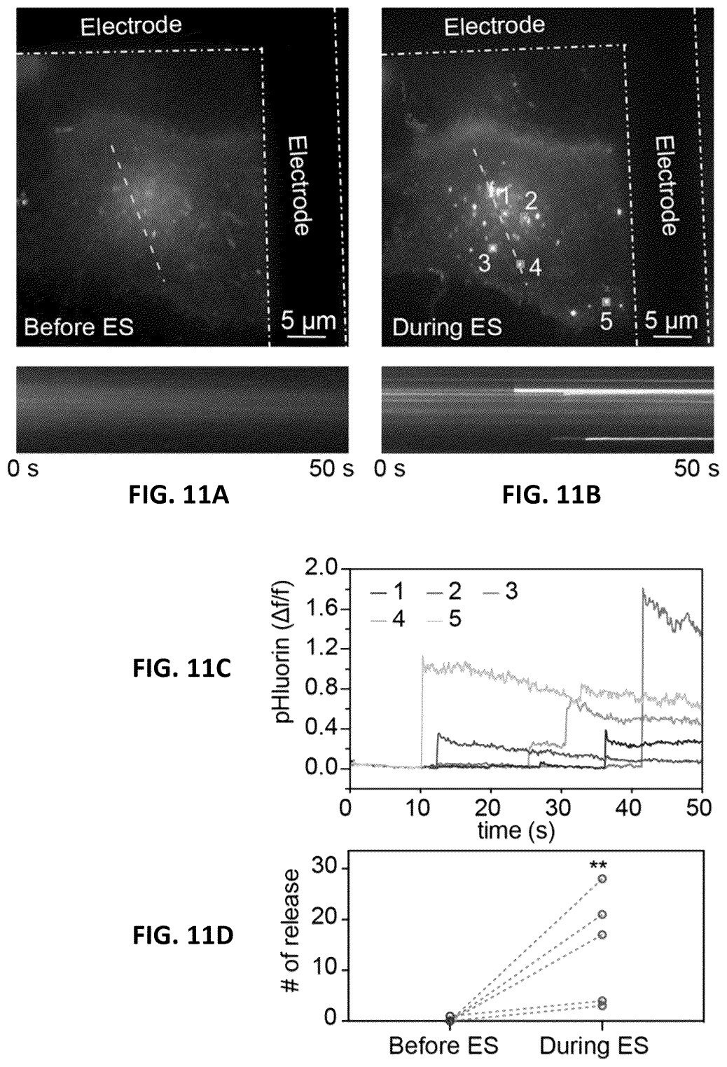

The voltage and the frequency of alternating fields were adjusted, and 1V, 2 Hz were identified as optimized bioelectrical stimulation conditions. Stimulation with ±2V, 2 Hz induces cell death and membrane leakage, indicating by the increased dead cell percentage upon raised voltages (FIG. 7), the elevated calcium levels (FIGS. 8A-8C), and the enlarged area of fluorescent pHluorin signal burst (FIG. 9A-9C). Upon bioelectrical stimulation of 1V for 0.25 s and −1V for 0.25 s (i.e., 1V, 2 Hz signals, FIG. 2C) for 50 s, the HeLa cell across the two interdigitated comb-shaped gold electrodes showed boosted exosomal secretion under super-resolution TIRF imaging, as shown in the pHluorin fluorescence signal burst (FIGS. 10A-10C and FIGS. 11A-11D). Therefore, to activate the MVB-PM fusion machinery and induce exosome release with minimal cell death, a stimulation of ±1V, 2 Hz was applied in subsequent experiments.

The exosomal release dynamics upon electrical stimulation in real-time was quantitatively assessed. Individual release events before and during electrical stimulation (±1V, 2 Hz) were counted in the same recording duration and the exosomal release signals were quantified (FIG. 11C-11D). Electrical stimulation significantly boosted exosomal releases in 50 seconds (FIG. 11C-11D). This discovery would be crucial for expanding exosome production for biomedical applications.

To further study the mechanisms that regulate exosome secretion, live-cell imaging and fixed-cell imaging were used to study actin cytoskeleton (FIG. 9), which is crucial for the study of many fundamental biological processes. Lifeact, a 17-amino-acid peptide that stained filamentous actin (F-actin) structures, was used to visualize the actin dynamics together with pHluorin-CD63 exosomal secretion monitor. AF647-phalloidin as also used to label F-actin and antiCD63 antibodies (AF555) to label EVs marker CD63 in fixed HeLa cells. Here, it is shown that actin cytoskeleton may control the trafficking of multivesicular endosomes (MVEs).

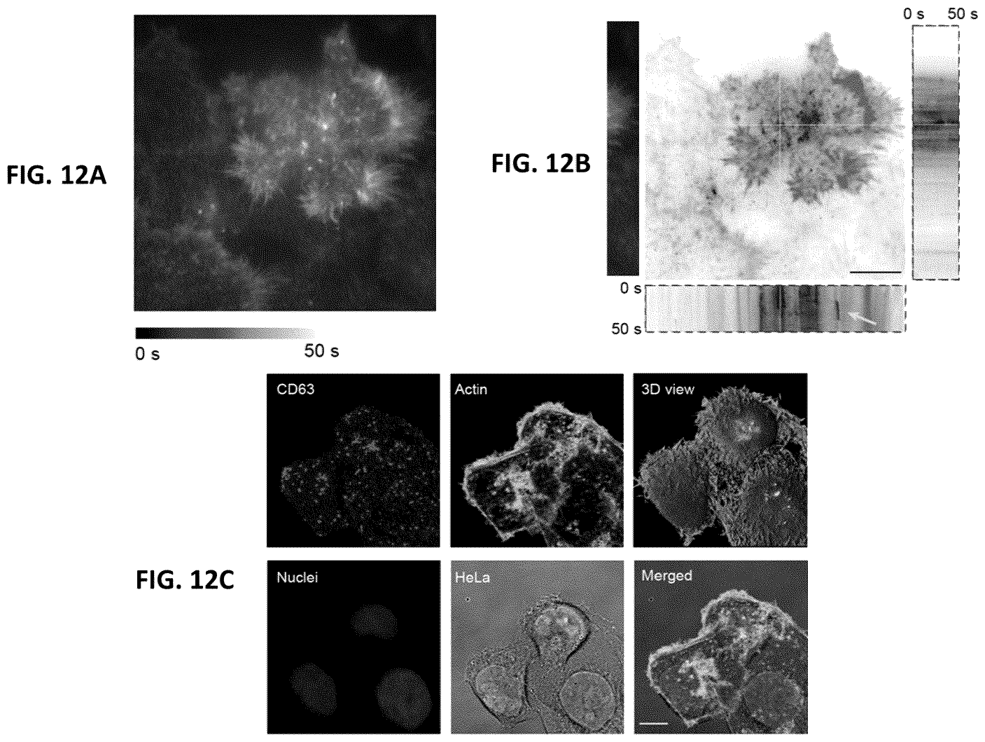

To further study the exosomam biogenesis under electrical stimulation, live-cell imaging and fixed-cell imaging were used to the actin filaments and CD63 exosome marke colocalization. Actin cytoskeleton is reported to play important roles in several fundamental biological processes, including controlling the trafficking of multivesicular endosomes (MVEs)^{26,33-35}. Lifeact, a 17-amino-acid peptide that stained filamentous actin (F-actin) structures, was used to visualize the actin dynamics together with pHluorin-CD63 (FIGS. 12A-12C), and used AF647-phalloidin was used to label F-actin and antiCD63 antibodies (AF555) to label EVs marker CD63 in fixed HeLa cells. It was shown that actin cytoskeleton aligned with CD63, the EVs marker, indicating actin coordinates the EVs release and membrane fusion, indicating that electrical stimulation-induced exosomal release followed predefined pathways of EVs occurrence where actin directs the transport and occurrence of vesicles releases.

### Example 4: EVs Characterization

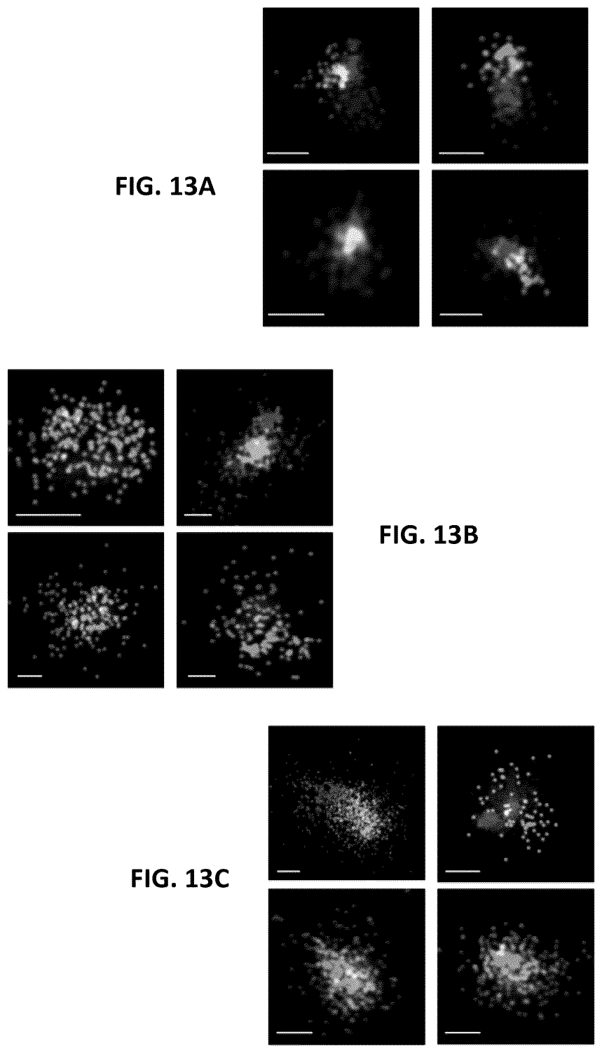

The bioelectrical stimulation-generated extracellular vesicles (e-EVs) were isolated and characterized. First, the subcellular structures and surface markers on e-EVs at the molecular scale were resolved using super-resolution direct stochastic optical reconstruction microscopy (dSTORM, FIG. 13). The multi-color dSTORM images verified the presence and spatial distribution of representative exosome markers (e.g., tetraspanins CD9, CD63, and CD81).

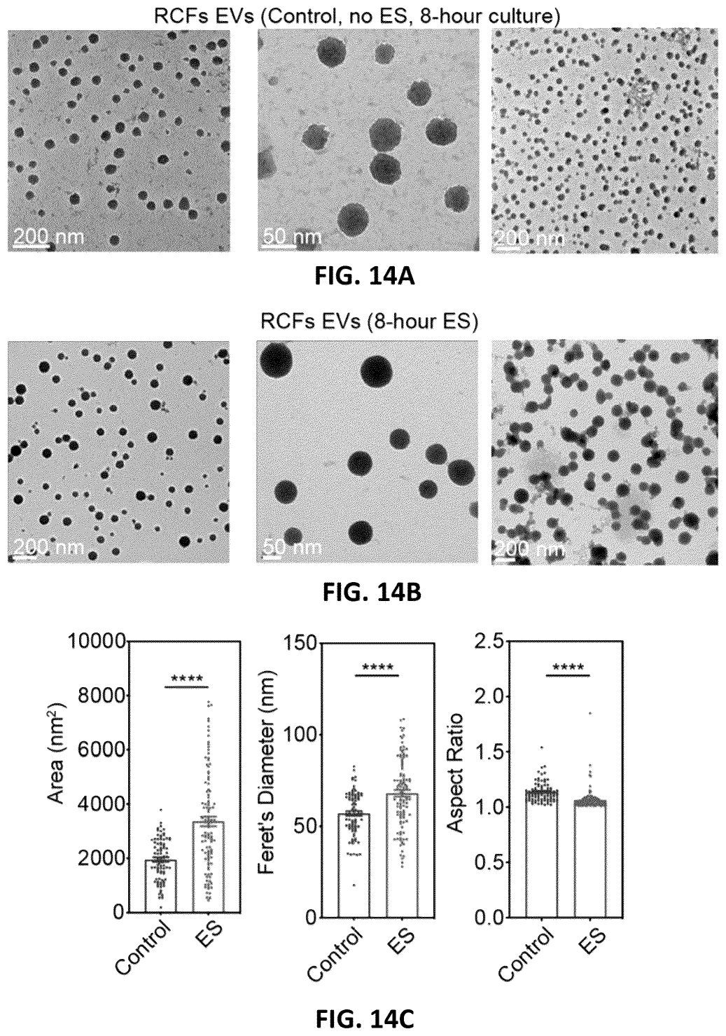

Additionally, transmission electron microscopy (TEM) imaging, the standard imaging technique in characterizing the nanoscale EVs, was used to resolve the e-EV sizes. EVs were collected and analyzed from cells without electrical stimulation operated in parallel together with the e-EVs characterization with the same culture, collection, dilution, and analysis procedures), and the e-EVs were found to be larger in size (FIGS. 14A-14C and FIGS. 15A-15D). It was found that e-EVs showed significantly larger sizes compared to EVs obtained from non-stimulated cells (verified with both RCFs and HeLa cells). Samples were prepared in parallel to minimize run-to-run variations.

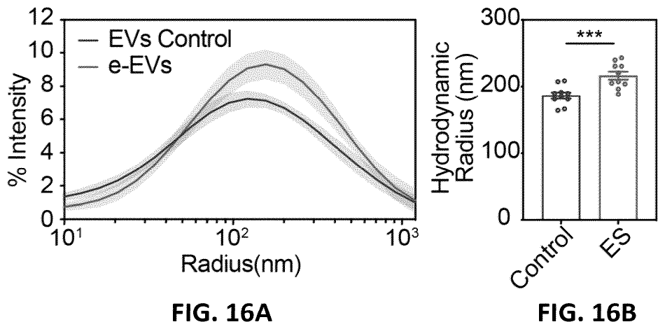

DLS measurements (FIG. 16A-16B) also showed that e-EVs samples has larger hydrodynamic radius than normal EVs. As a result, the increased size of e-EVs would further help EVs engineering such as encoding therapeutical cargos by allowing for a higher loading capacity.

### Example 5: Scaling Up the Production of Electrical Generated EVs (e-EVs)

Using EVs as microRNA carriers is minimally invasive to biological systems as EVs are endogenous biological signals. Besides, EV membranes enable stable preservation of the cargo microRNAs to avoid degradation and digestion in biofluid.

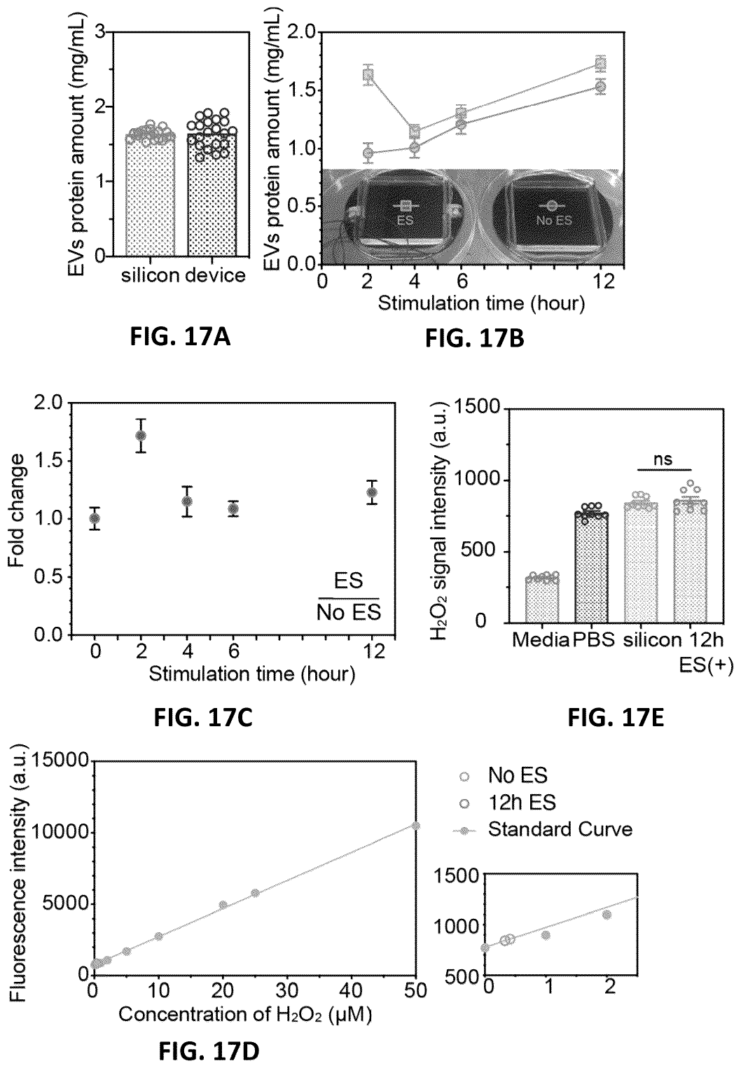

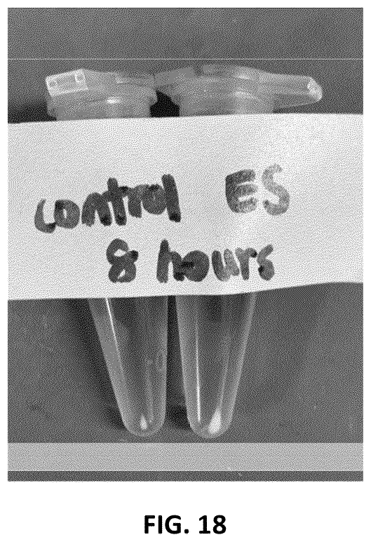

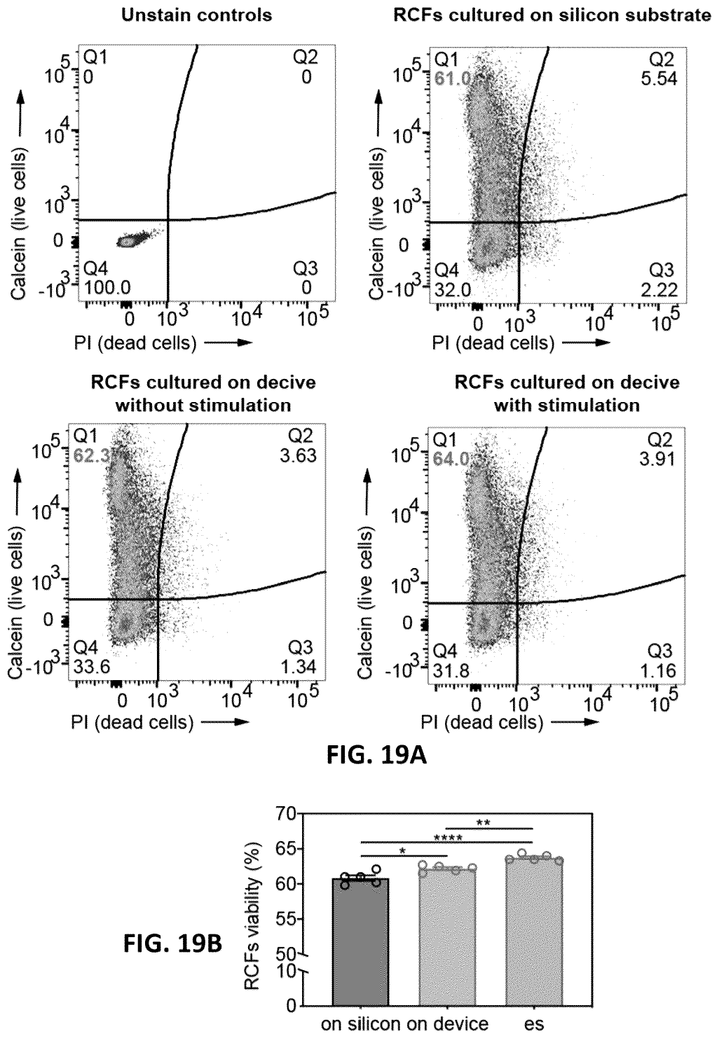

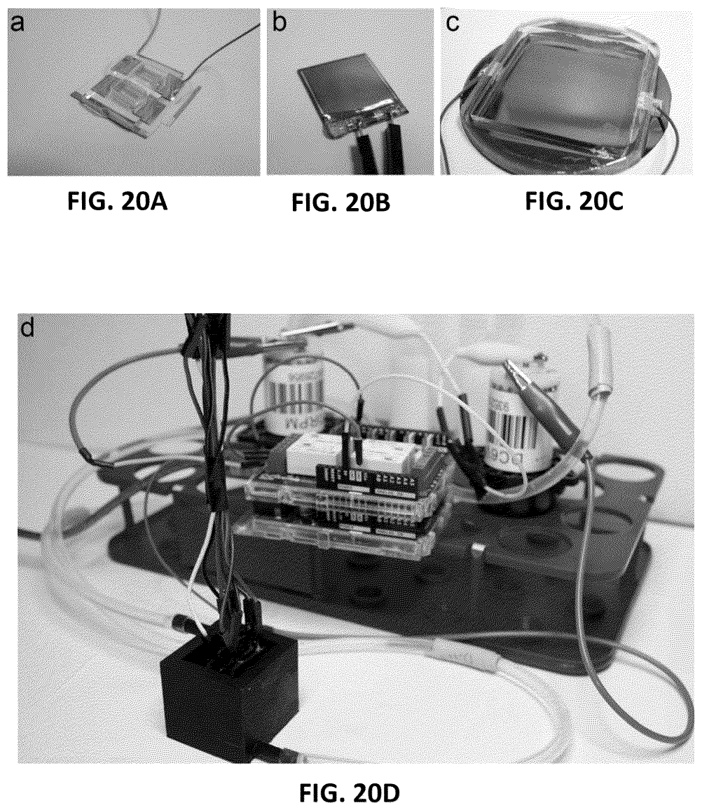

As a proof-of-concept study to demonstrate the usage of electrical stimulation produced EVs (E-EVs) as shuttle for therapeutical microRNAs delivery, the bioelectrical stimulation devices were first scaled up on large piece silicon wafer to produce sufficient homogeneous quantities of EVs as the therapeutical drug delivery building block (FIG. 17A-17E). The isolated EVs from electrically stimulated (8-hour) HeLa cells were significantly more than the amount from non-stimulated HeLa (FIG. 18). Thus, sufficient homogeneous quantities of EVs were produced as the therapeutical agent delivery building blocks. Cell remained viable after 24-hour long-period electrical stimulation on the large-scale devices (FIG. 19A-19B). Alternatively, a continuous flow system (FIG. 20D) can also be used to scale up the e-EVs production for further expanded production.

### Example 6: Representative Device Fabrication and Continuous Flow Systems

Fabrication of a representative embodiment of the device in combination with a continuous flow system to collect the extracellular vesicles produced by cells is described below.

Interdigited bioreactor electrodes. Device design was performed in AutoCAD software, and schematics of the interdigitated devices are shown in FIG. 20A-20D. Electrodes had a width of 15 μm with 10 μm spacing between them and 300 μm distance to the edge of the comb. The single-channel device was composed of 150 pairs of electrodes connected to two large pads, allowing for manual connection to jumper wires.

Interdigitated gold electrodes were fabricated using photolithography on either silicon wafers for high-throughput exosome production or #1.5 glass substrates for super-resolution imaging. Electrode patterns were exposed using Heidelberg direct writer and developed by AZ 300MIF using AZ2020 photoresist. 5 m chromium and 100 nm gold were evaporated on the patterned surface using an electron-beam evaporator. Lastly, the resist was stripped in remover PG. To form the cell-culture well on the electrode, a mixture of 10:1 PDMS to curing agent was molded and bonded to the electrode surface.

Continuous flow exosome harvesting systems. An Arduino Uno microcontroller was connected with a four relays shield, which can be then used to control the on and off of the two peristaltic pumps. One pump is used to transport liquid from delivery tube to the black 3D printed culturing chamber through the small tubing design on the top right side. Another pump is used to transport liquid from culturing chamber to collection tube through the small tubing design on the bottom left side. The 3D printed black chamber is designed to contain multiple chips. The whole system is controlled with the Arduino IDE on the computer to achieve programmable media delivery, transportation, and collection.

### Example 7: Engineering Electrical Generated EVs for Cardiac Tissue Repair

Proliferation of cardiomyocytes after damage show potential in promoting cardiac repair⁴⁴⁻⁴⁶. Several microRNAs that are responsible for cardiomyocyte proliferation are of increasing interest to prompt cardioprotective strategies⁴⁷⁻⁵¹, for example, a miR-199a was shown to stimulate adult mouse cardiomyocytes to enter the cell cycle and proliferate following myocardial infarction (MI)^{47,52-54}. MiR-199a regulates critical pathways in cardiomyocytes glucose metabolism, hypertrophy, apoptosis, and autophagy⁵⁵⁻⁵⁷, and induce pathological fibrosis in fibroblasts^{52,58}. Other studies have identified that miR210^{59,60} promotes angiogenesis in acute myocardial infarction.

As a proof-of-concept study to demonstrate the usage of electrical stimulation produced EVs (e-EVs) as shuttle for therapeutical microRNAs delivery to repair cardiac function, the e-EVs production, isolated e-EVs, loaded e-EVs with the therapeutical miR199a-3p and miR210 (1:1 mixture, miRNAs-E-EVs) were scaled up, and miRNAs-E-EVs were used to treat mice with acute myocardial infraction (AMI).

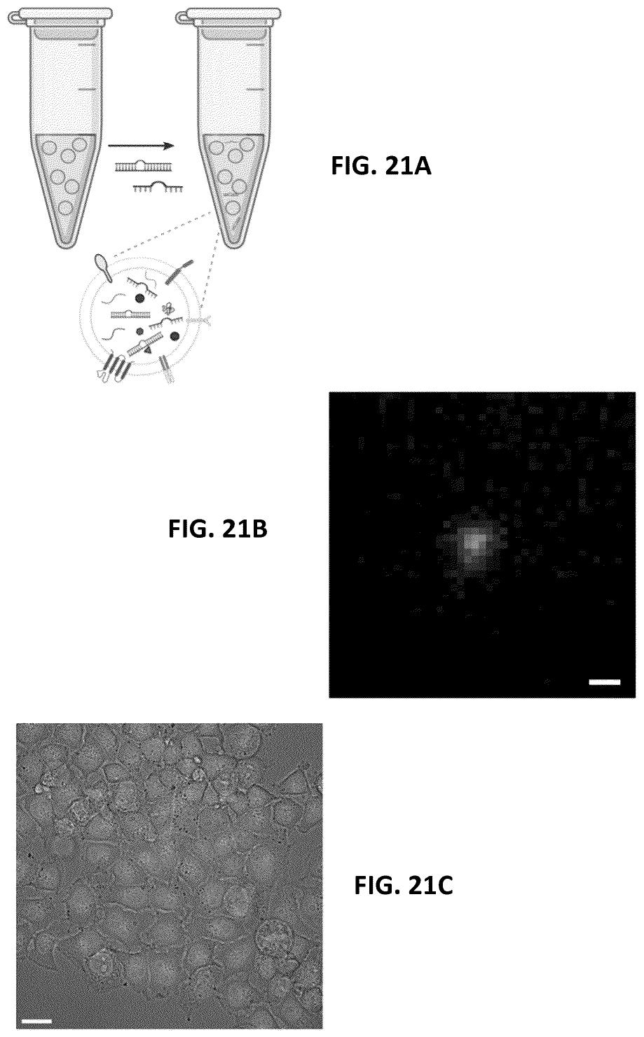

Primary rat cardiac fibroblasts (RCFs) were cultured with electrical stimulation on the device on a 4-inch silicon substrate. RCFs were used as it was reported to carry endogenous microRNAs that are responsible for cardiomyocyte proliferation to promote cardiac repair. The EVs were isolated from electrically stimulated (12-hour) RCFs supernatant (DMEM with 10% exosome-depleted FBS). The therapeutical miR199a-3p and miR210 (1:1 mixture) were loaded into the isolated e-EVs (miRNAs-e-EVs) using transfection method according to manufacturer's instructions and recommended doses. Successful loading was verified with fluorescence-labelled siRNA as positive loading control (FIG. 21A-21C).

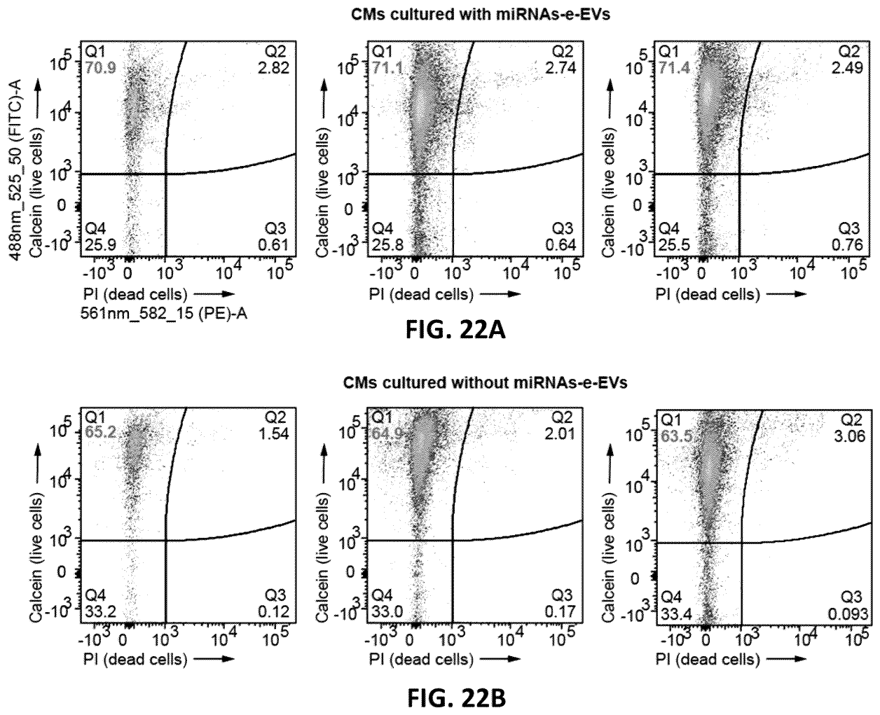

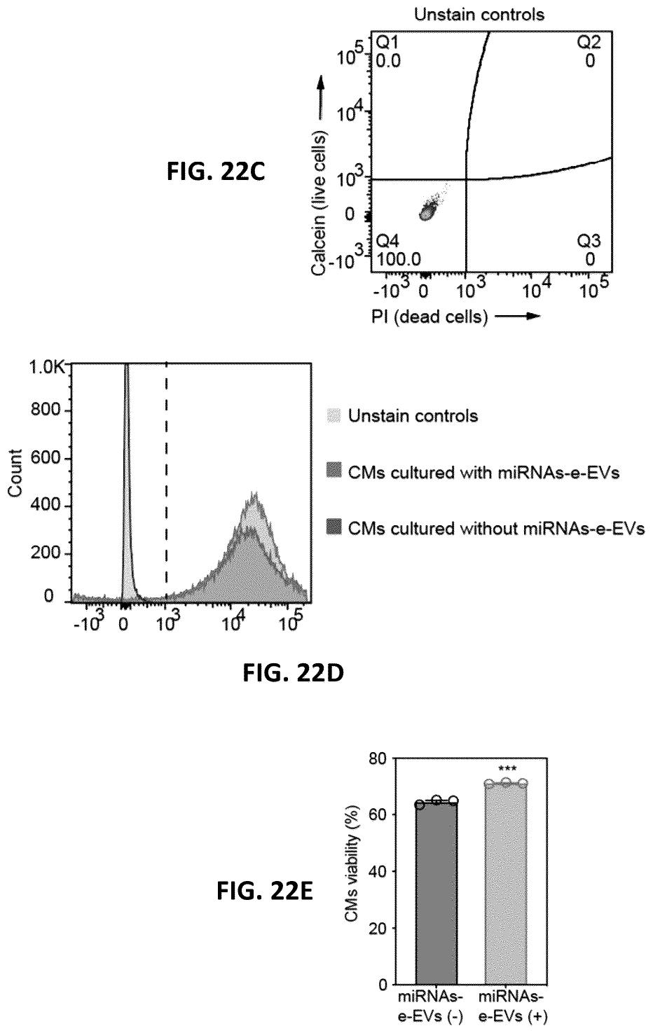

It was hypothesized that miRNAs-e-EVs treatment can empower the endogenous capacity of cardiac repair after damage by promoting regeneration of lost contractile tissue. To test this hypothesis in vitro, primary rat cardiomyocytes (CMs) were incubated with prepared miRNAs-e-EVs for 12 hours. By assessing the CMs viability using flow cytometry, it was found that miRNAs-e-EVs promoted CMs survival within 12 hours (FIG. 22A-22E).

### Example 8: In Vivo Myocardial Infarction Treatment with miRNAs-E-EVs

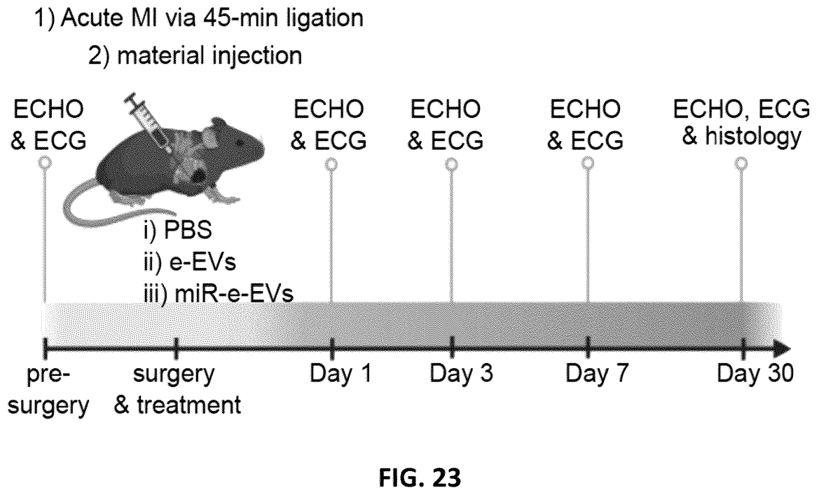

Cardiac function repair was evaluated in rodent models of myocardial infraction (MI) using wild-type C57B16 mice (6-8 weeks of age, male, weight>20 g). During the MI surgery, the left anterior descending coronary artery (LAD) was ligated for 45 minutes, after which PBS solution (negative control), e-EVs, and therapeutical e-EVs with cardiac specific miRNAs-e-EVs were delivered, respectively, to the mice MI hearts, by four intramyocardial injections in four locations (10 μL each) to peri-infarct area within 5 minutes after reperfusion into each mouse. In this experimental setting, the delivery of the samples directly following MI surgery avoids a second surgery, which would importantly increase animal mortality. Treatment efficiency assessment was performed using echocardiography (echo), electrocardiogram (ECG), and histological phenotypic characterization (FIG. 23).

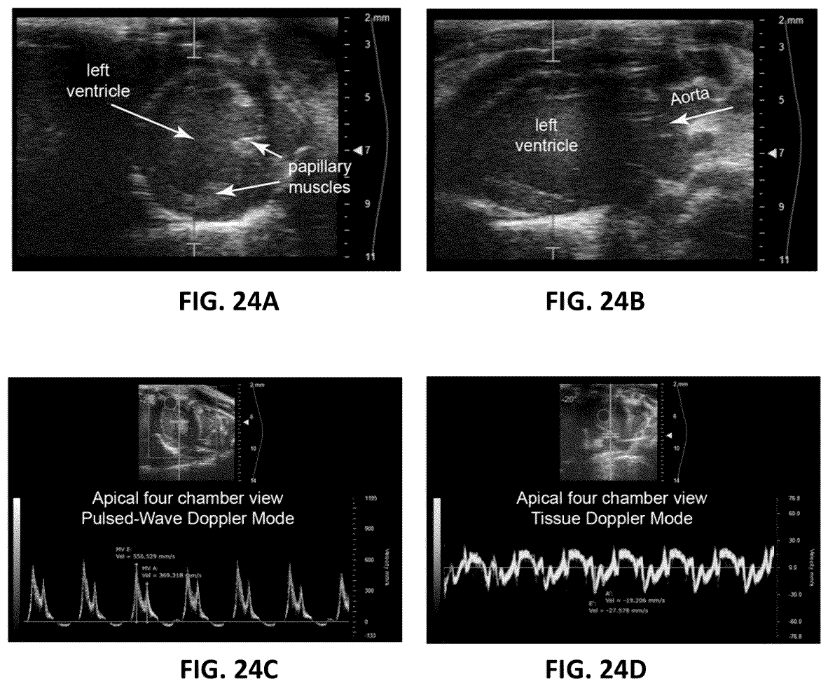

During echo, electrocardiogram (ECG) signals and heart rates were recorded and studied. To identify the anatomical structures and evaluate the cardiac systole and diastole functions, parasternal long-axis view (PLAX, FIG. 24A) and parasternal short-axis view (PSAX, FIG. 24B) were collected using motion mode (M-mode) setting and the left ventricular (LV) ejection fraction (EF), fraction shortening (FS), and cardiac output (CO), stroke volume, and LV mass were measured. Besides, in the apical four chamber view, pulsed-wave (pw) doppler mode (FIG. 24C) and tissue doppler mode (FIG. 24D) waveforms were collected to assess mitral valve flow and annulus, where the mitral valve E wave, A wave, e′ velocity, and a′ velocity were measured and E/A and E/e′ ratios were studied to evaluate the systolic and diastolic function and myocardium health.

### Example 9: Cardiac Function Repair in Myocardial Infarction Mice

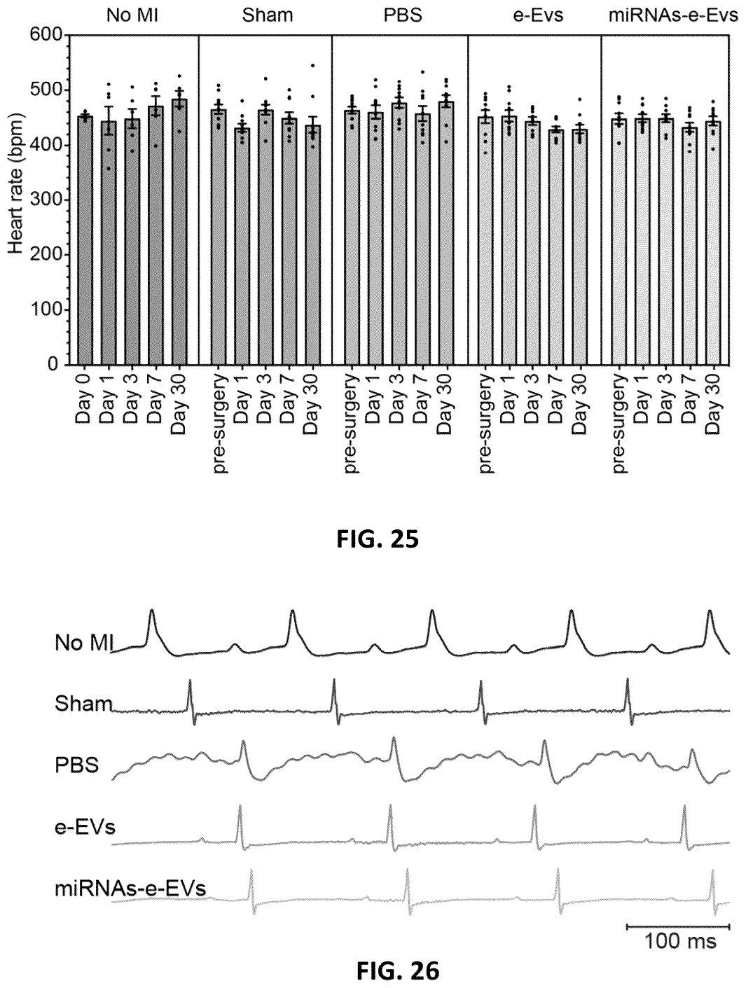

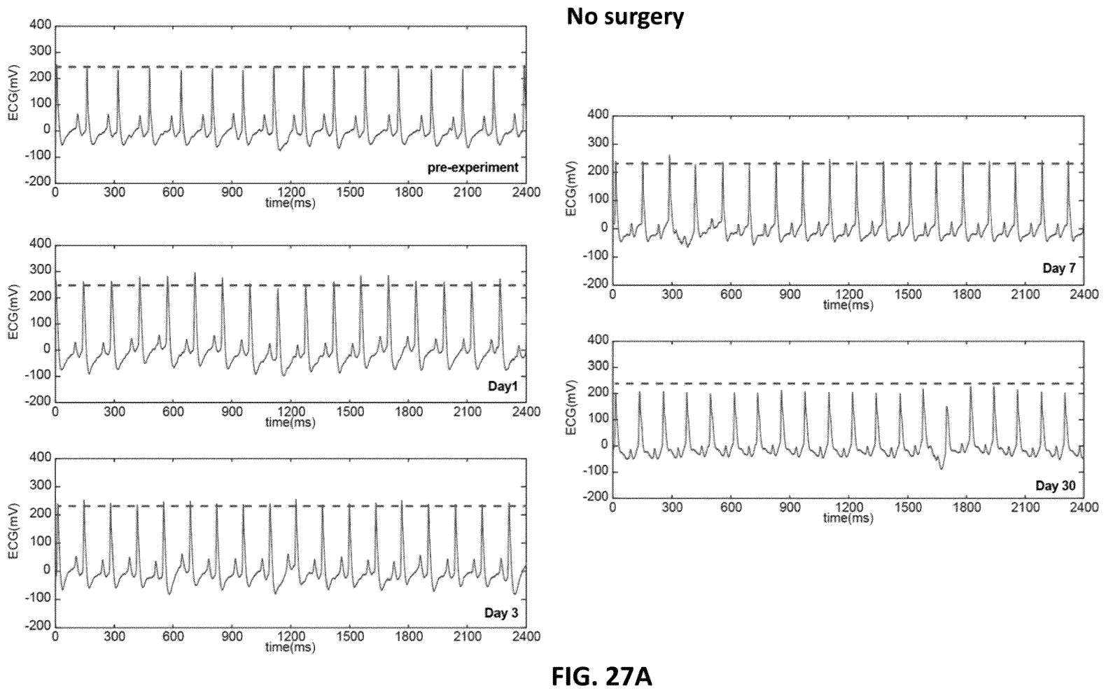

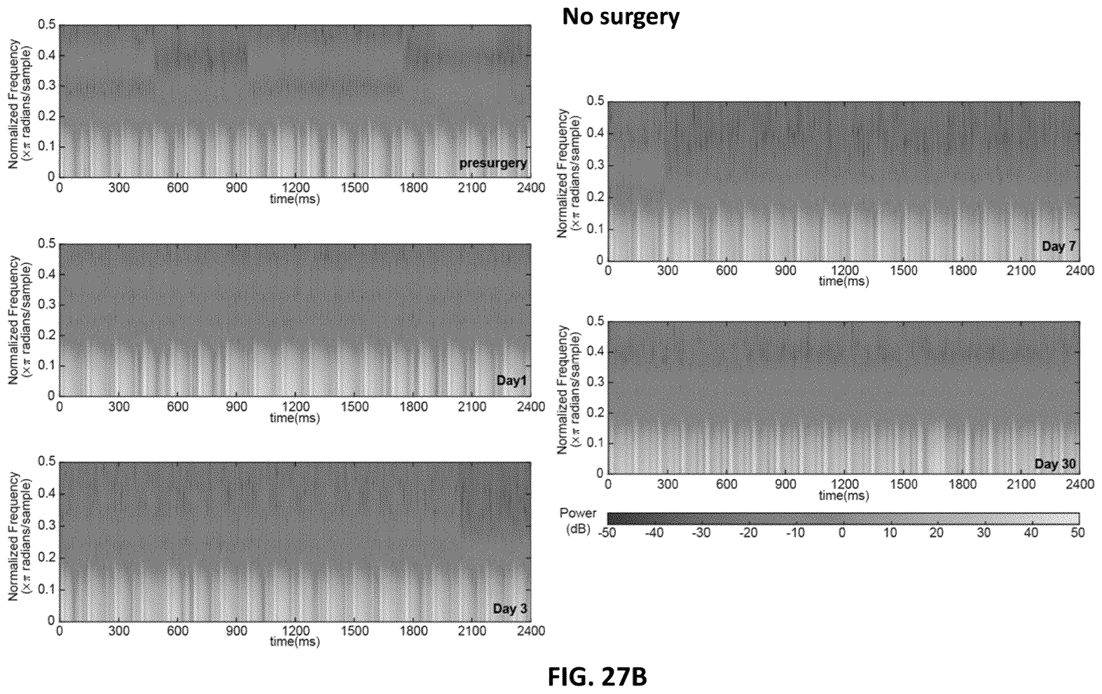

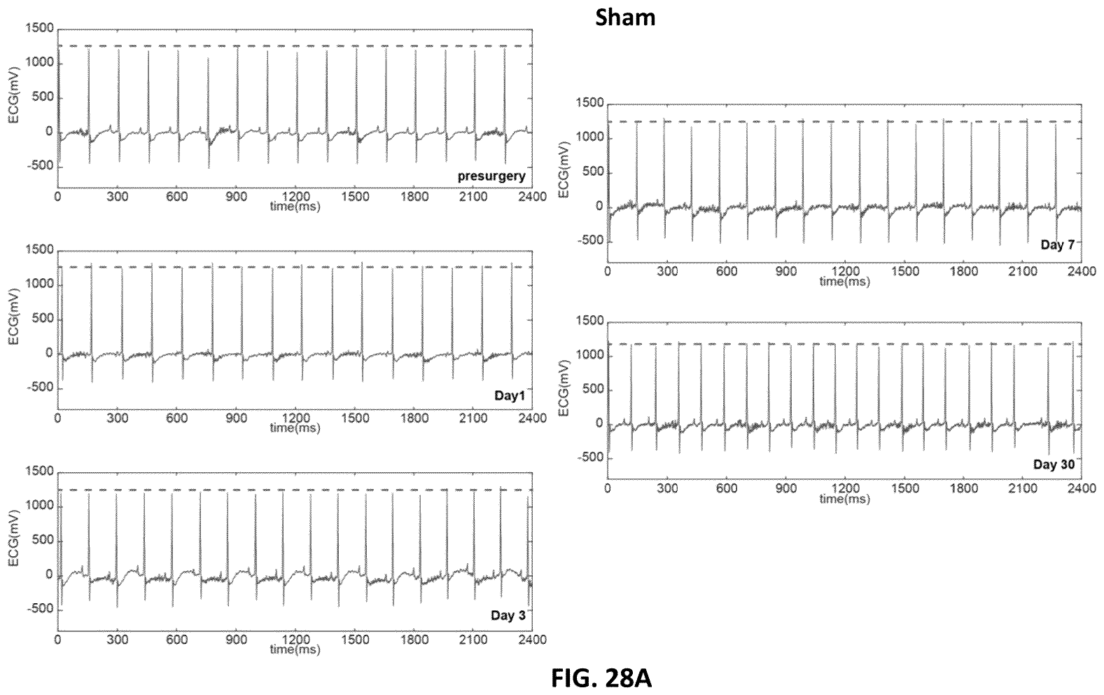

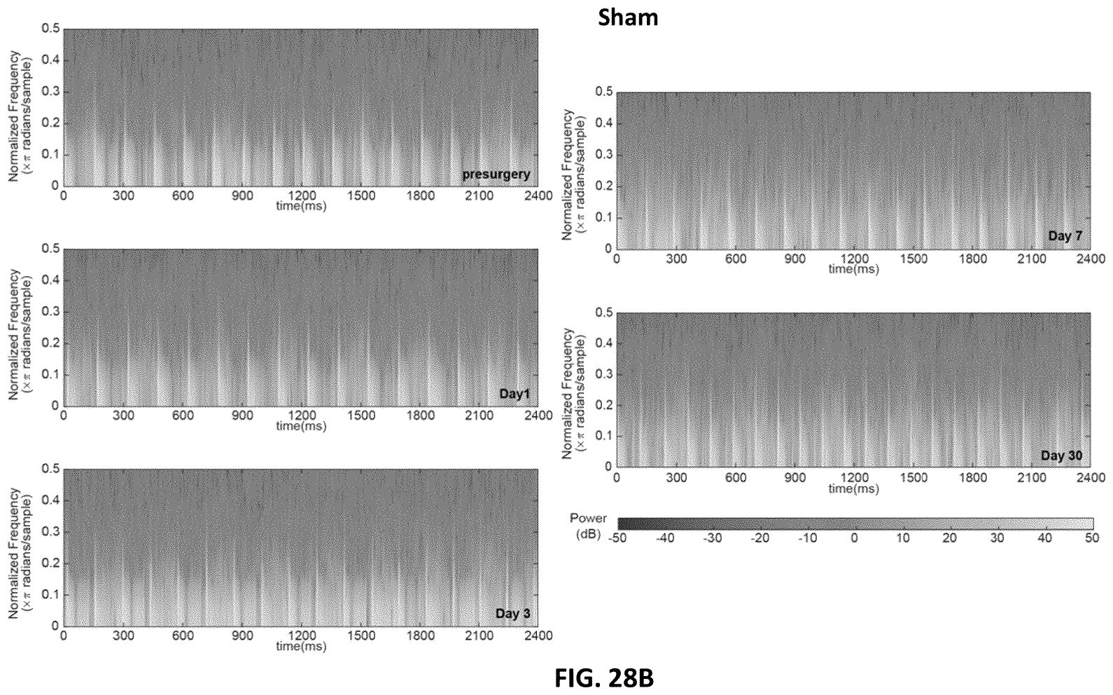

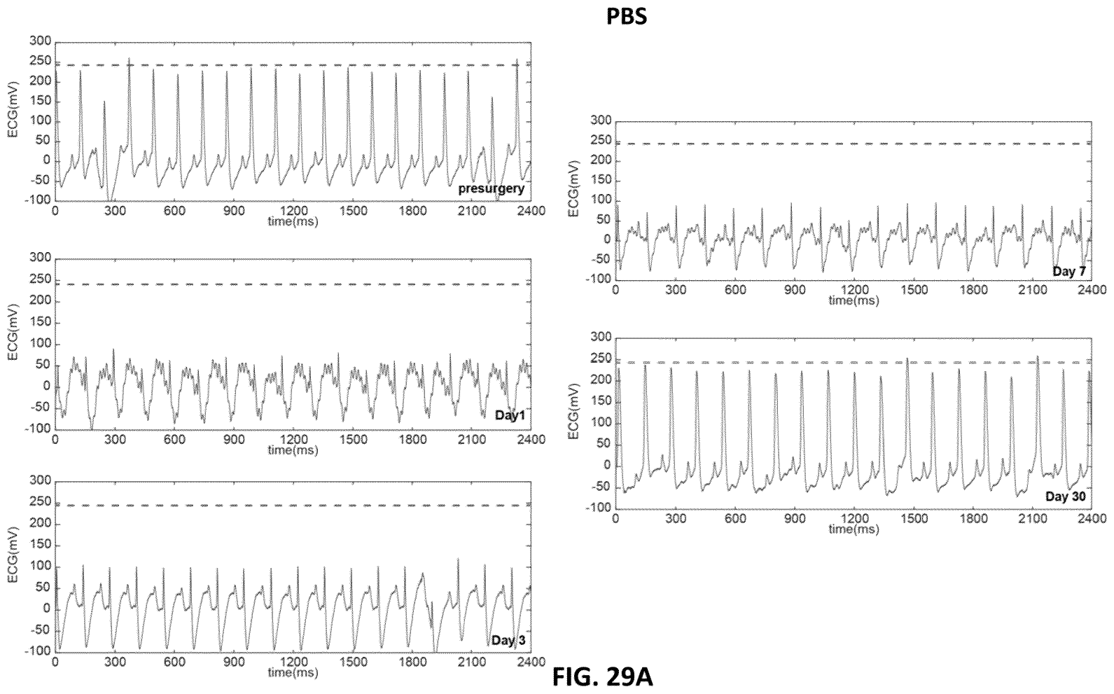

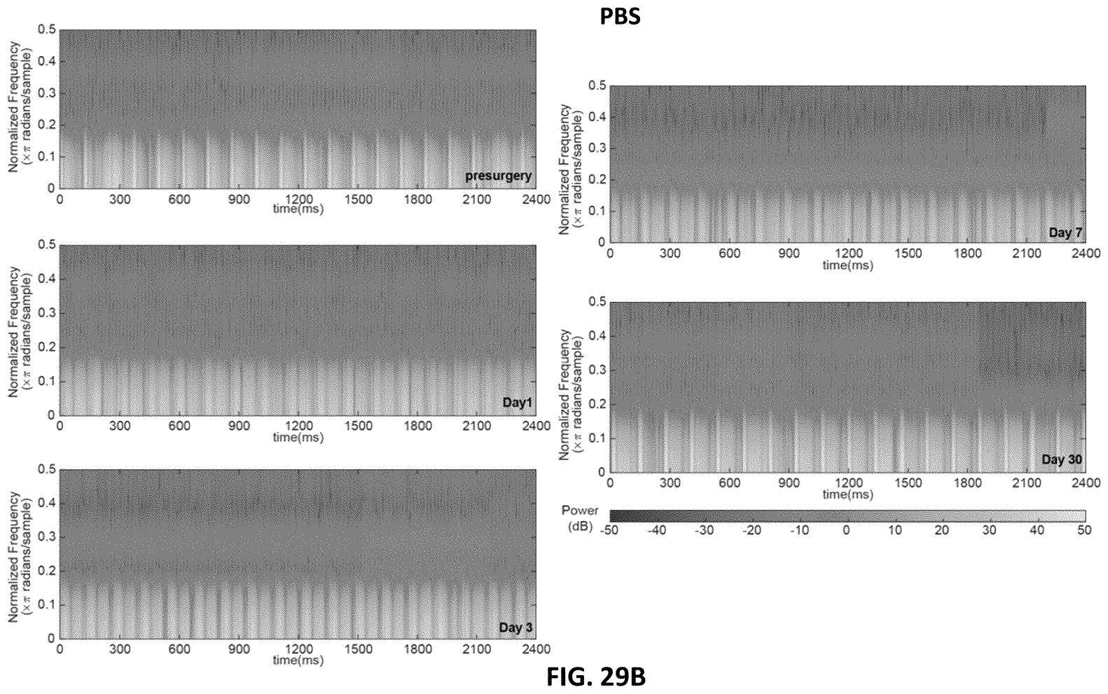

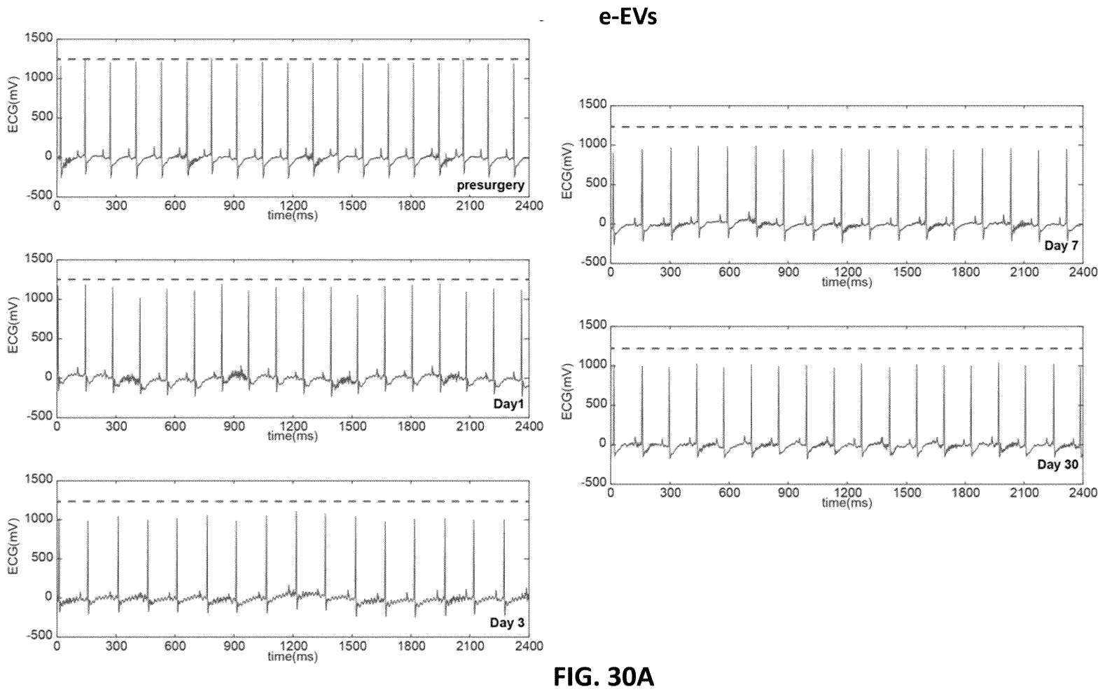

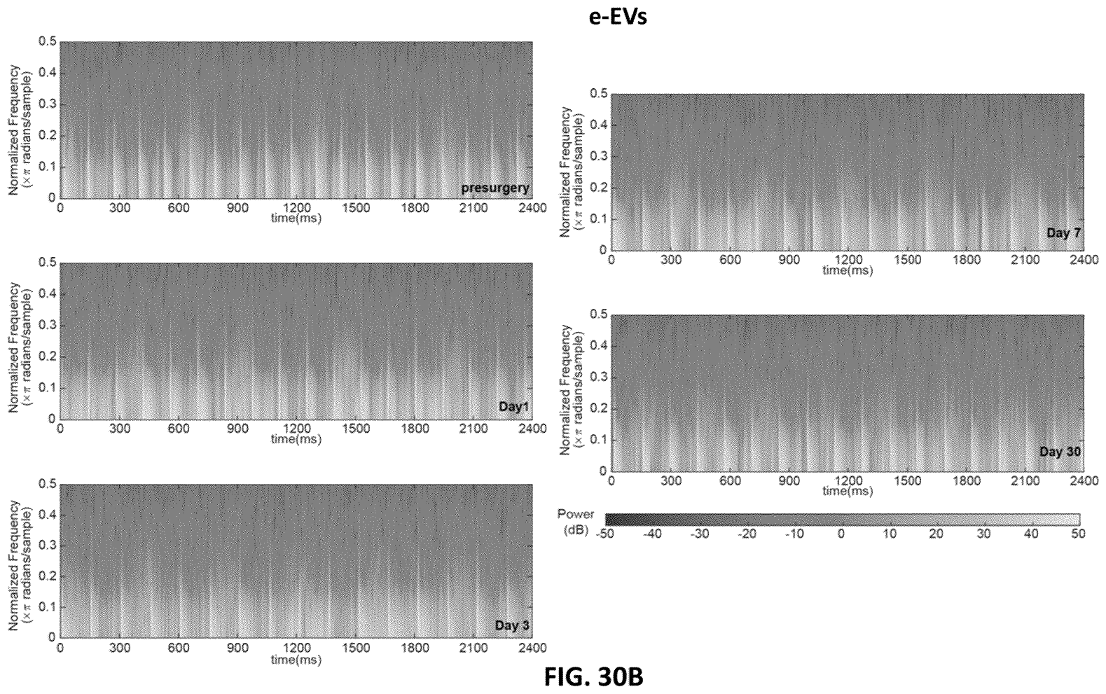

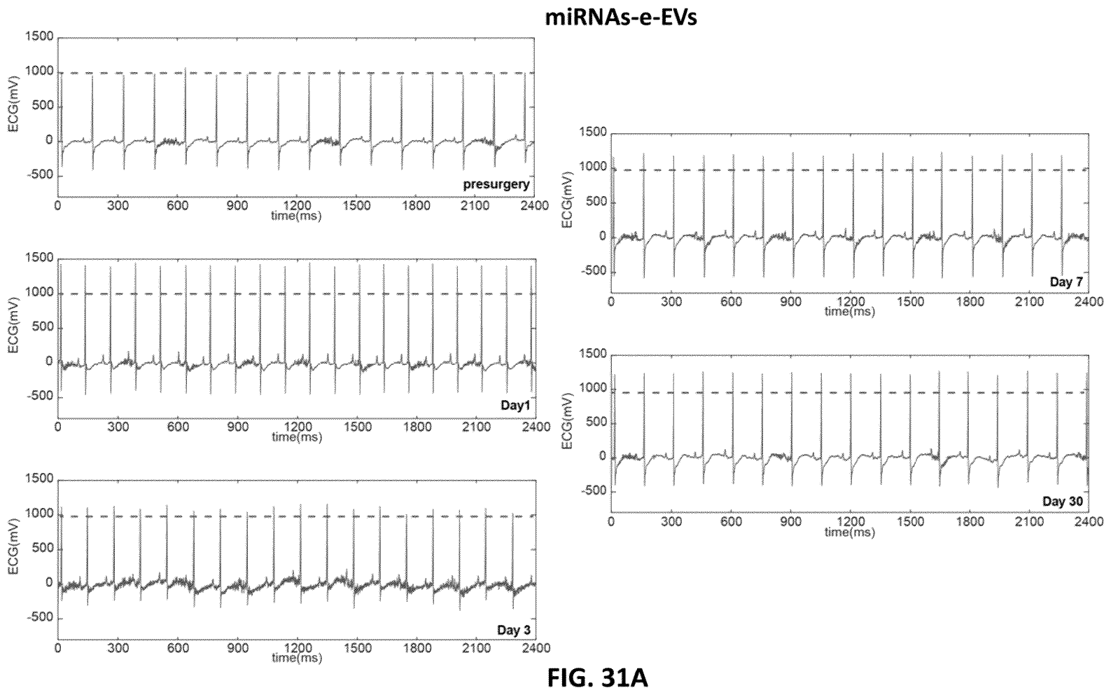

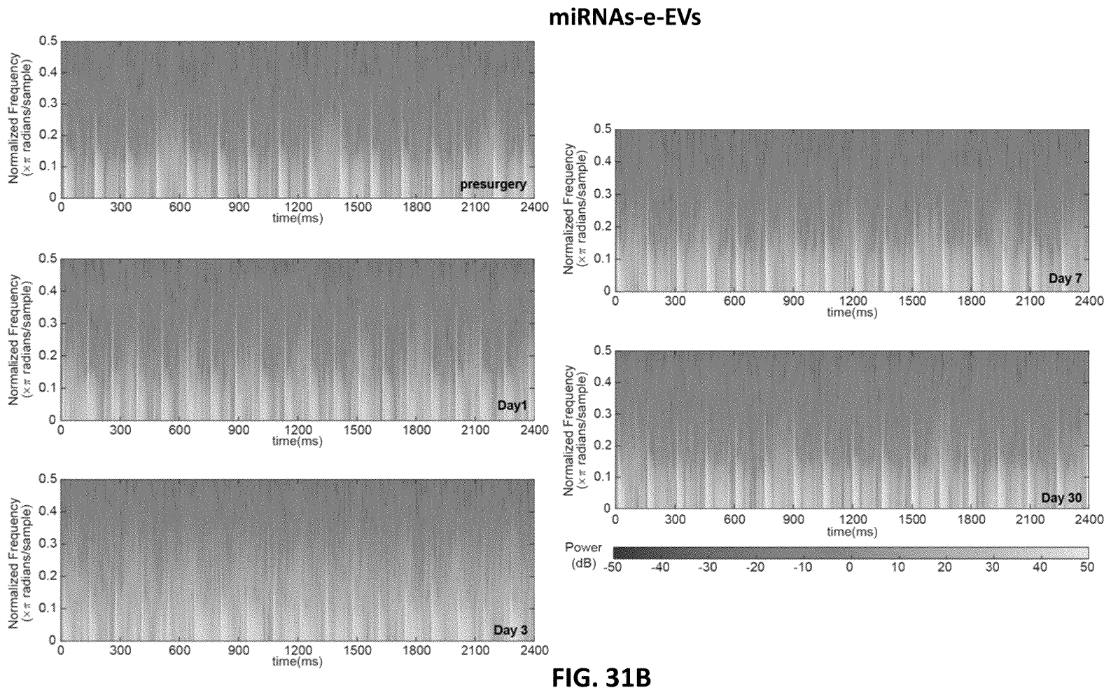

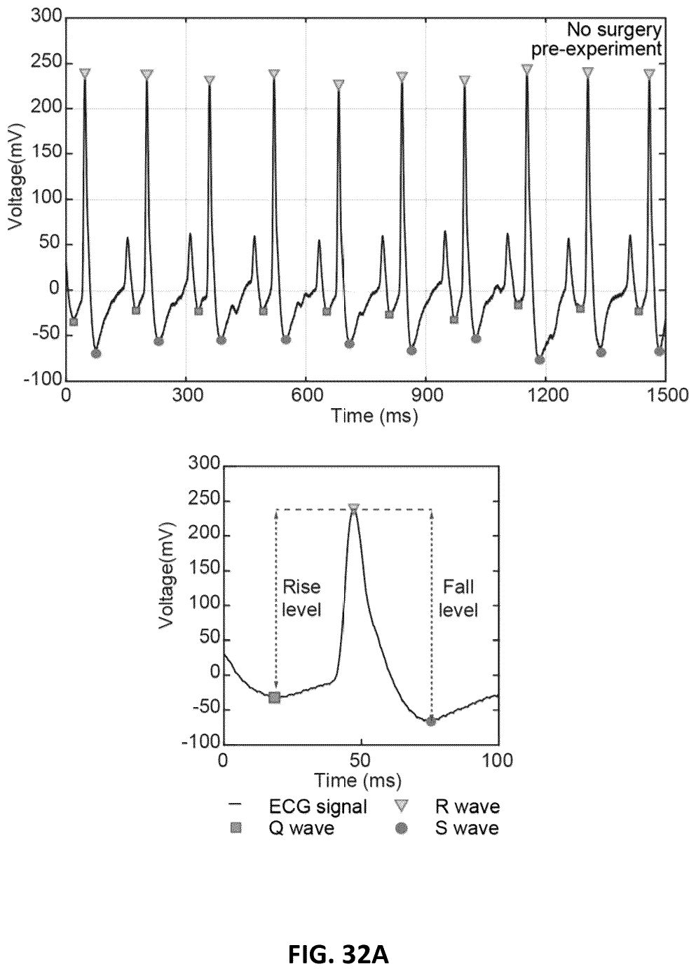

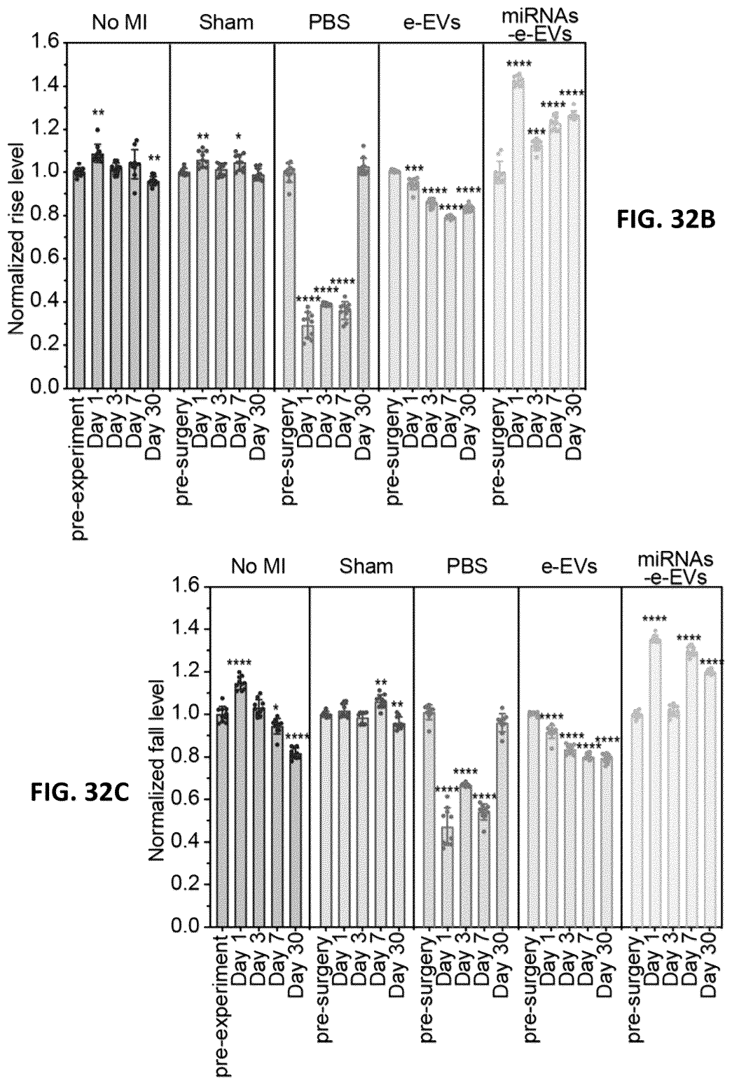

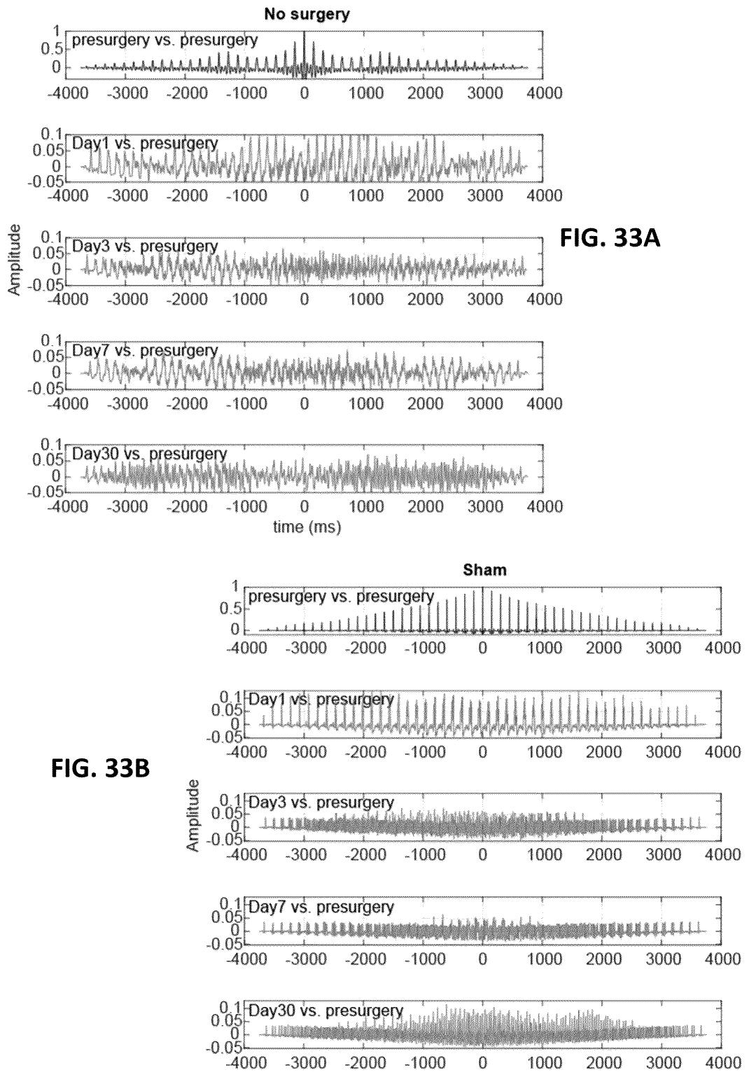

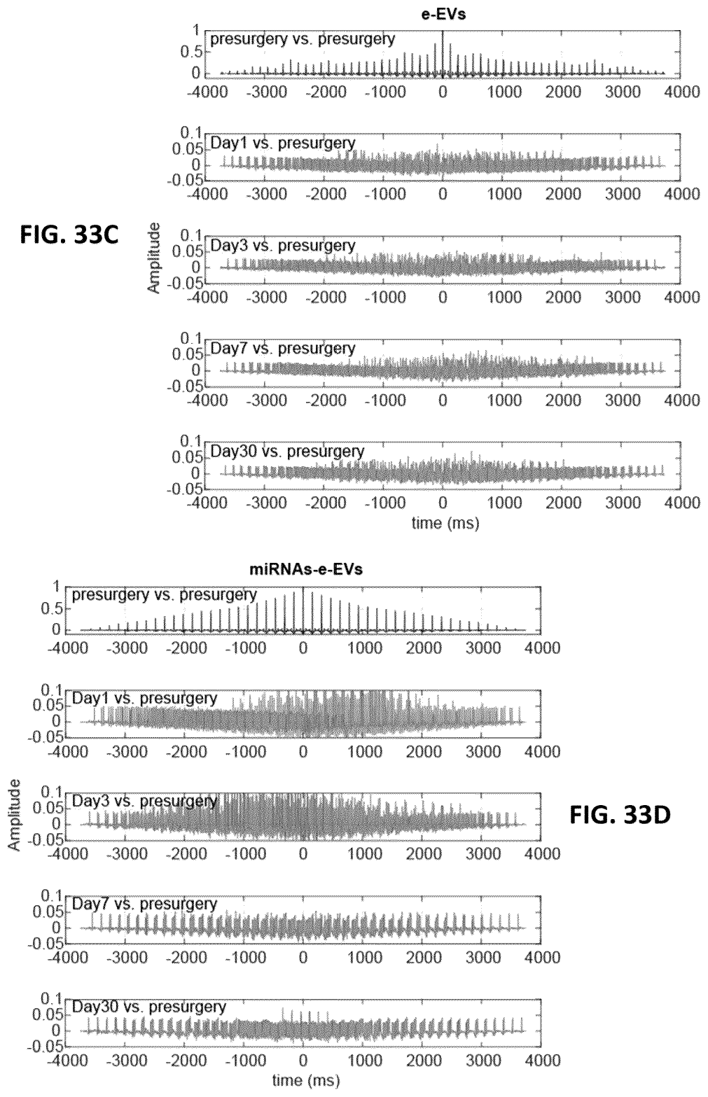

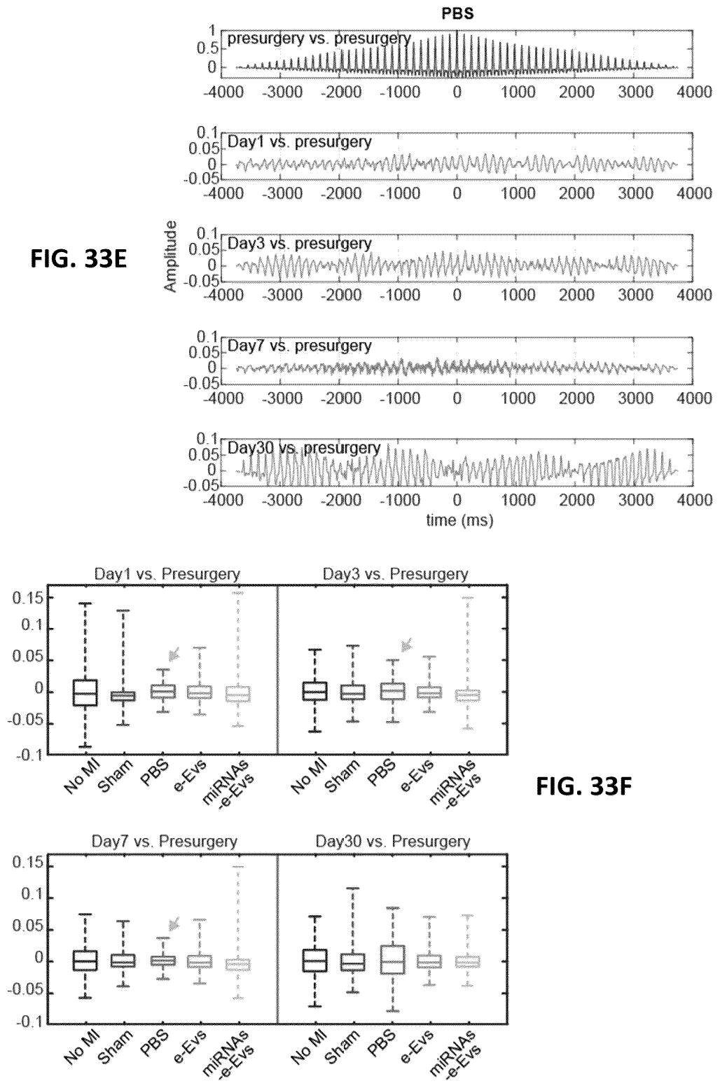

The recorded electrocardiogram (ECG) showed similar heart rates (FIG. 25) and heart rhythms (FIG. 26) between e-EVs treatment and sham treatment mice. The sham treatment mice means only open chest surgery was performed without ligation (or myocardial infarction) on the heart. While ECG remained similar to pre-surgery (healthy) state in no surgery group, sham group, and two e-EVs treatment groups (FIG. 27A to FIG. 31B), PBS-treated MI mice showed abnormal electrical activity of the heart (FIG. 26), diminished ECG level (˜only 40% retention of rise rates and ˜60% retention of fall rates, FIGS. 29A-29B and FIGS. 32A-32C), lowest ECG signal similarity (FIG. 33A-33F) compared to pre-surgery health state.

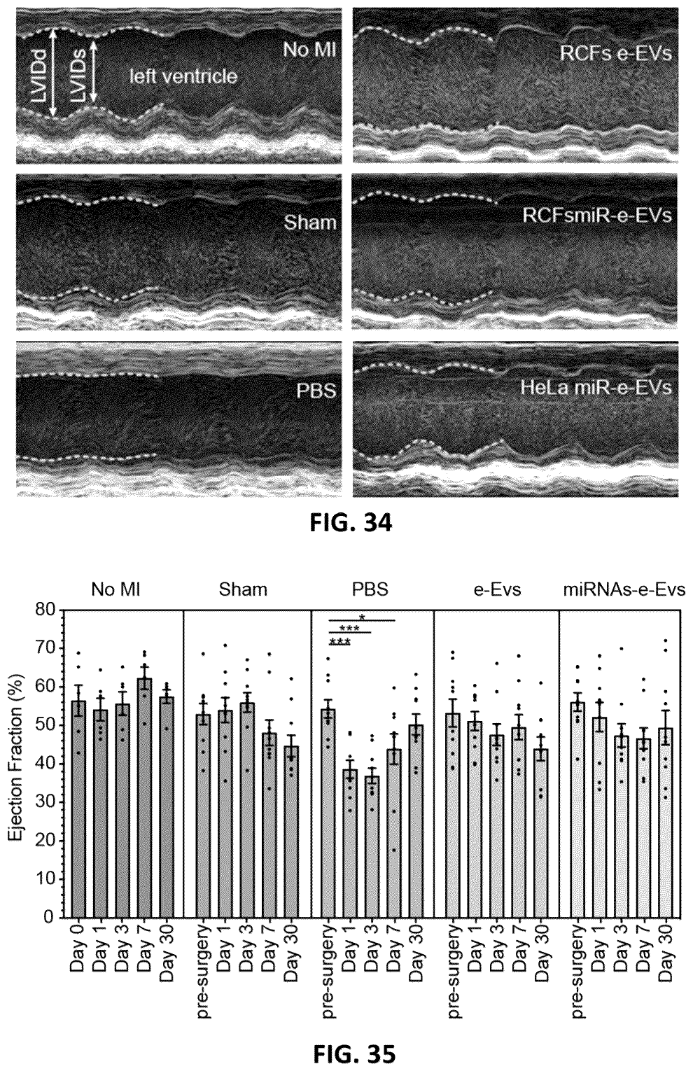

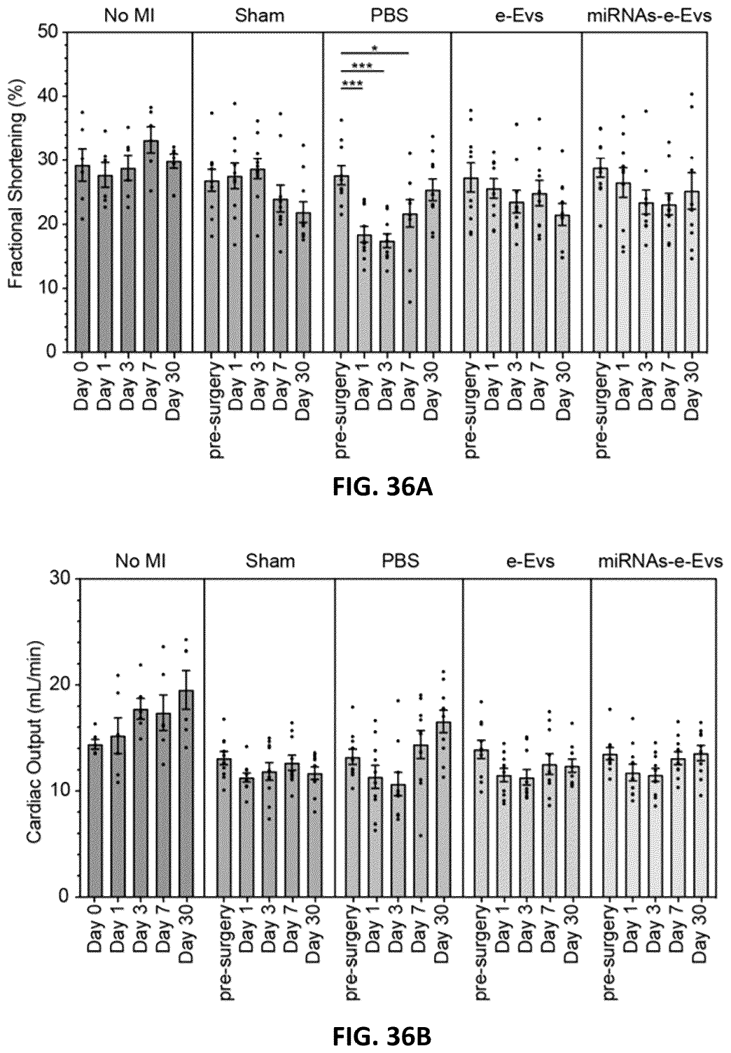

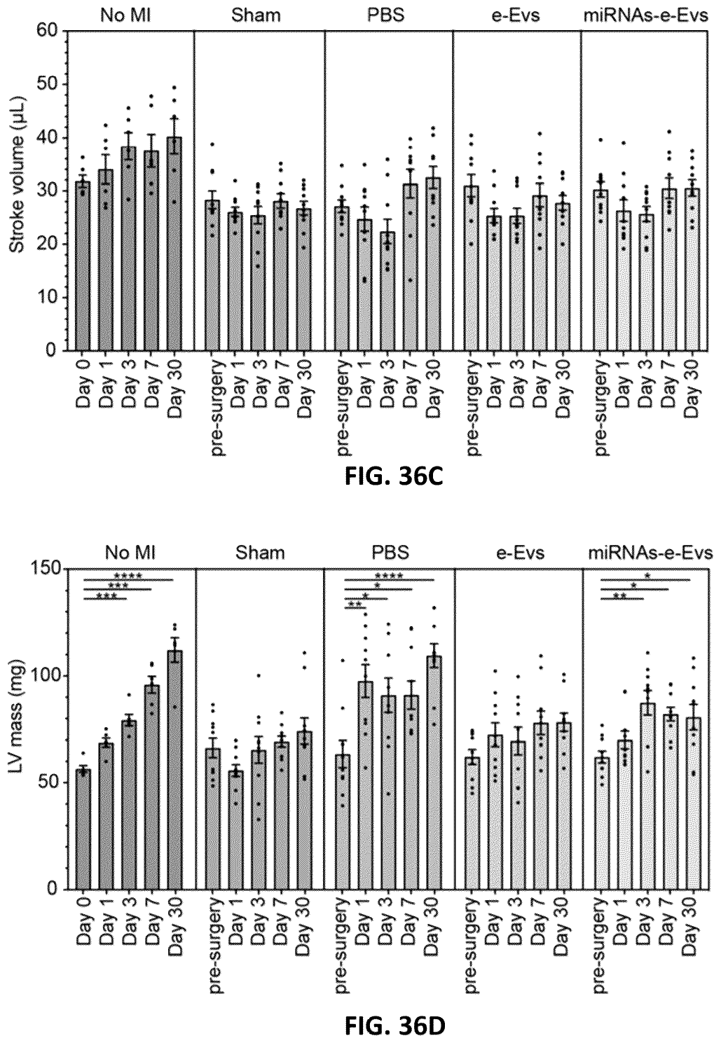

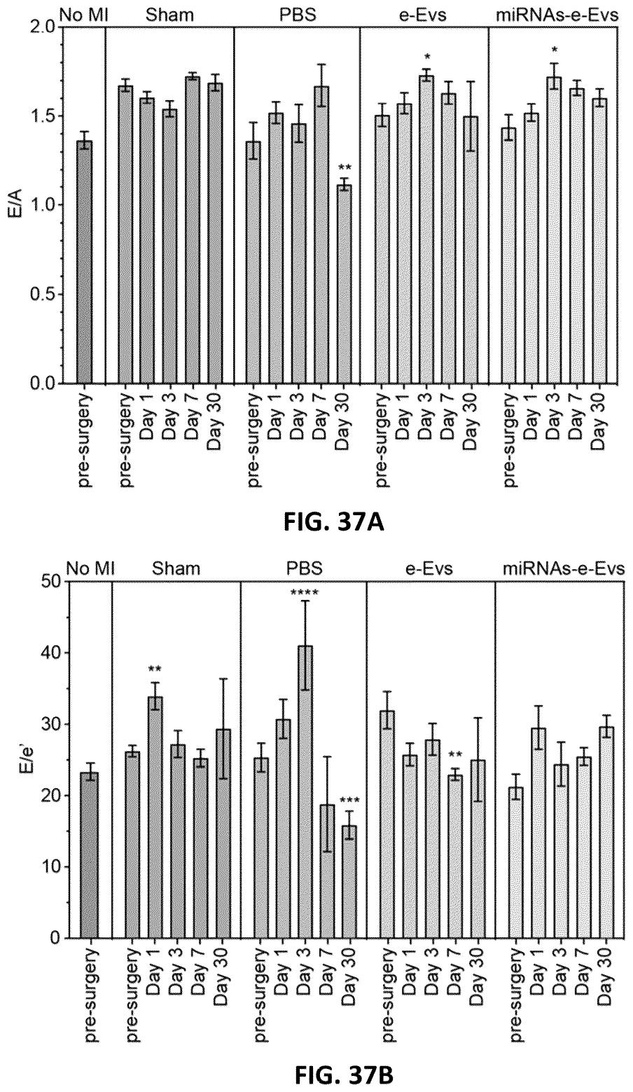

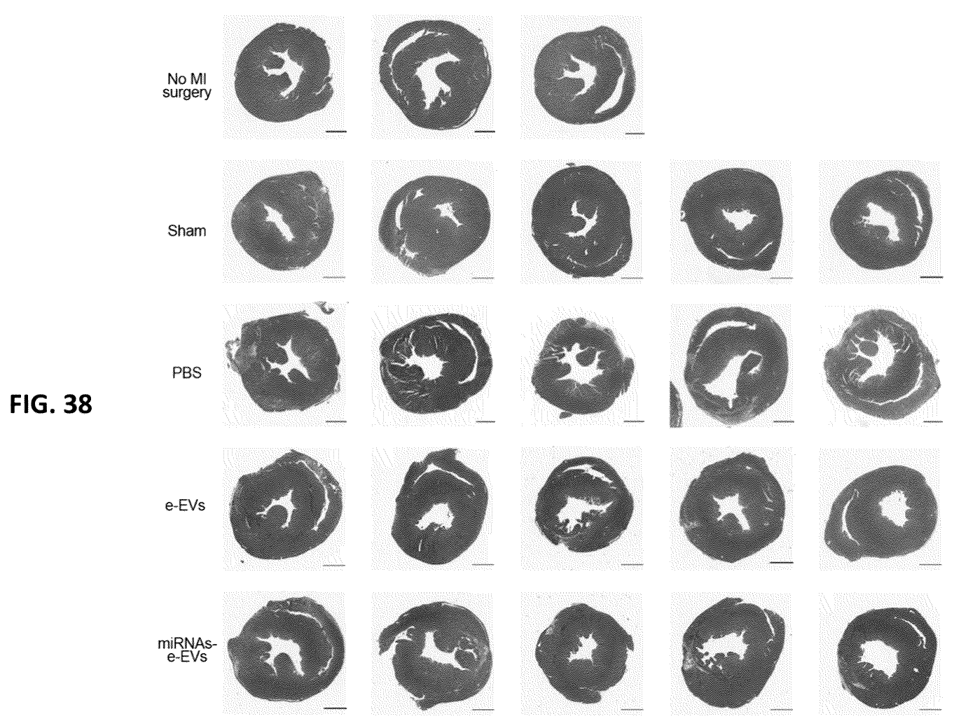

By evaluating left-ventricular (LV) functions using ECHO and histological phenotypic characterization, e-EVs loaded with or without miRNAs demonstrated improved heart diastole and systole cycles (FIG. 34) compared to PBS-treated MI mice (negative control). Quantitative and statistical studies of ejection fraction (EF, FIG. 35) showed that e-EVs loaded with or without miRNAs both demonstrated EF similar to pre-surgery or no surgery EF values (no significance observed in pairwise comparison, p>0.05). However, MI mice with no therapeutical treatment (i.e., PBS treated) showed impaired ventricular filling and contraction/ejection (as presented with significantly reduced EF values, FIG. 35). Besides, fraction shortening (FS), and cardiac output (CO), stroke volume, LV mass, E/A ratio, and E/e′ ratio studies all demonstrated that e-EVs improved the heart function compared to negative control (FIGS. 36A-36D and 37A-37B).

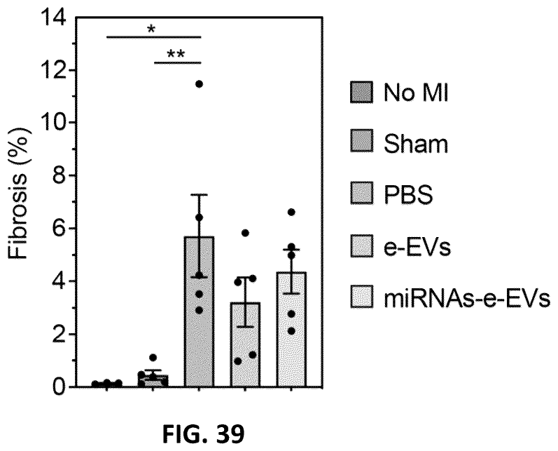

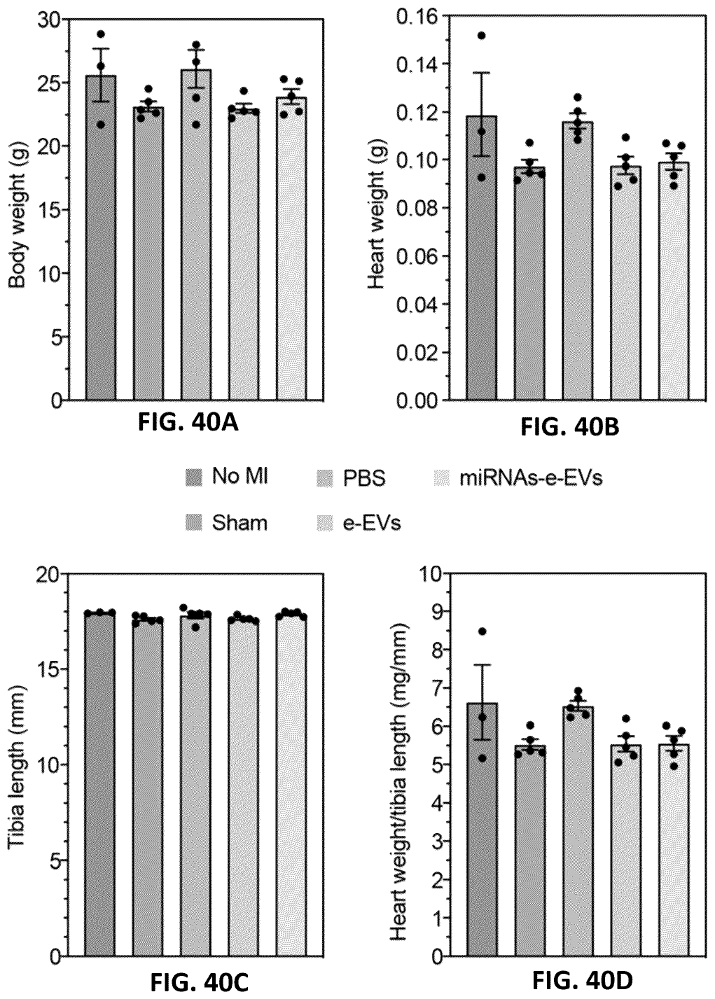

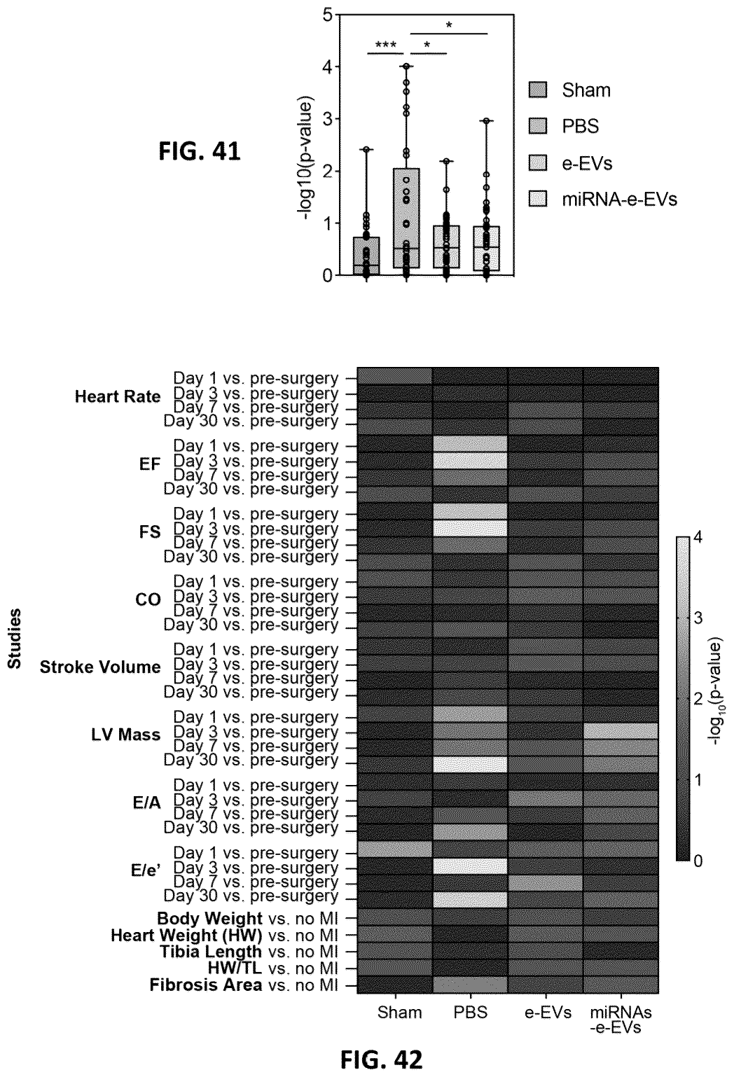

After 30 days survival, phenotypic characterization of the heart was conducted via histological Masson's trichrome (MTC) staining and cardiac fibrosis was quantified. Treatment of miRNAs-e-EVs and e-EVs both showed relative low fibrosis post-MI compared to the PBS negative control (FIG. 38 and FIG. 39), indicating e-EVs and miRNAs-e-EVS provided therapeutical effect that further preventing heart fibrosis. The heart weight, body weight, and tibia lengths of the MI mice were also measured after 30-day survival post-MI, where no significant difference in mouse growth was observed (FIG. 40A-40D). Overall, a summary of statistical and quantitative results of all the animal studies (Table 1, FIGS. 41 and 42) showed the electrical stimulated-RCFs-generated e-EVs can help treating mouse MI without developing cardiac systolic and diastolic failure.

TABLE 1
Statistical p values in mice MI model studies.

| | | | | | miRNA-e- |
|---|---|---|---|---|---|
| Study | Comparison | Sham | PBS | e-EVs | EVS |
| Heart rate | Day 1 vs pre-surgery | 0.0829 | 0.9983 | 0.9794 | >0.9999 |
| | Day 3 vs pre-surgery | >0.9999 | 0.785 | 0.9093 | >0.9999 |
| | Day 7 vs pre-surgery | 0.65 | 0.9852 | 0.18 | 0.4675 |
| | Day 30 vs pre-surgery | 0.1781 | 0.6675 | 0.2 | 0.9889 |
| EF | Day 1 vs pre-surgery | 0.9971 | 0.0008 | >0.9999 | 0.8287 |
| | Day 3 vs pre-surgery | 0.878 | 0.0002 | 0.5199 | 0.2198 |
| | Day 7 vs pre-surgery | 0.5911 | 0.0352 | 0.8203 | 0.1634 |
| | Day 30 vs pre-surgery | 0.1614 | 0.6808 | 0.1305 | 0.4419 |
| FS | Day 1 vs pre-surgery | 0.9963 | 0.0006 | 0.9993 | 0.8481 |
| | Day 3 vs pre-surgery | 0.8878 | 0.0001 | 0.4475 | 0.2239 |
| | Day 7 vs pre-surgery | 0.6459 | 0.0381 | 0.7789 | 0.1816 |
| | Day 30 vs pre-surgery | 0.1863 | 0.7039 | 0.1211 | 0.5561 |
| CO | Day 1 vs pre-surgery | 0.1671 | 0.5476 | 0.1093 | 0.1957 |
| | Day 3 vs pre-surgery | 0.4679 | 0.2826 | 0.0724 | 0.123 |
| | Day 7 vs pre-surgery | 0.9717 | 0.8627 | 0.5536 | 0.9745 |
| | Day 30 vs pre-surgery | 0.3618 | 0.1073 | 0.454 | >0.9999 |
| Stroke volume | Day 1 vs pre-surgery | 0.6097 | 0.8434 | 0.1175 | 0.2899 |
| | Day 3 vs pre-surgery | 0.4027 | 0.3503 | 0.0838 | 0.1809 |
| | Day 7 vs pre-surgery | 0.9999 | 0.4478 | 0.8825 | 0.9999 |
| | Day 30 vs pre-surgery | 0.8394 | 0.2445 | 0.5004 | 0.9998 |
| LV mass | Day 1 vs pre-surgery | 0.3296 | 0.0041 | 0.2983 | 0.5472 |
| | Day 3 vs pre-surgery | 0.9997 | 0.0255 | 0.6971 | 0.0011 |
| | Day 7 vs pre-surgery | 0.9759 | 0.0254 | 0.1071 | 0.0116 |
| | Day 30 vs pre-surgery | 0.5845 | <.0001 | 0.0984 | 0.021 |
| E/A | Day 1 vs pre-surgery | 0.8080 | 0.4651 | 0.8705 | 0.7426 |
| | Day 3 vs pre-surgery | 0.3413 | 0.8766 | 0.0231 | 0.0406 |
| | Day 7 vs pre-surgery | 0.9089 | 0.1007 | 0.4662 | 0.0567 |
| | Day 30 vs pre-surgery | 0.9999 | 0.0050 | >0.9999 | 0.2493 |
| Ele' | Day 1 vs pre-surgery | 0.0038 | 0.3084 | 0.0696 | 0.0513 |
| | Day 3 vs pre-surgery | 0.9880 | <0.0001 | 0.4094 | 0.7508 |
| | Day 7 vs pre-surgery | 0.9871 | 0.5056 | 0.0064 | 0.4436 |
| | Day 30 vs pre-surgery | 0.8882 | 0.0003 | 0.2551 | 0.0594 |
| Body weight | vs. No MI | 0.1672 | 0.4370 | 0.1361 | 0.4323 |
| Heart weight (HW) | vs. No MI | 0.0703 | 0.9998 | 0.0771 | 0.1088 |
| Tibia length (TL) | vs. No MI | 0.1192 | 0.7886 | 0.1600 | 0.9669 |
| HW/TL | vs. No MI | 0.1030 | 0.9983 | 0.1098 | 0.1162 |
| Fibrosis area | vs. No MI | 0.9996 | 0.0152 | 0.3101 | 0.0876 |

### Discussion and Outlook

The bioelectrical stimulation on exosomal production was investigated, and it was found that exposing cells to alternating electrical stimulation stimulates their generation without detriment to cell viability. Here, planar 2D interdigitated gold electrodes as electronic stimulation devices were fabricated to deliver low-voltage, low-frequency (e.g., 1V, 2 Hz) electrical signals. Real-time EVs dynamics in live cells upon the bioelectrical stimulation were simultaneously studied using super-resolution total internal reflection fluorescence (TIRF) microscopy with the help of pHluorin-tagged CD63 exosome markers, which make the release events visible under super-resolution microscope. Quantified exosomal production events revealed the amplified or boosted EVs production upon low-voltage, low-frequency electrical stimulation. Given the high post-stimulation cell viabilities (>90%), cells can be recycled for iterative culture and bioelectrical stimulation, which facilitate a high throughput production of a homogeneous population of exosomes—a particular challenge for translating EVs therapy into clinical practice. Given the large sizes of electrical generated EVs (e-EVs) quantified from transmission electron microscope (TEM) imaging, e-EVs can load more biopharmaceutical cargoes, making it more beneficial as delivery carriers.

As a prove-of-concept demonstration, the e-EVs production were scaled to generate homogeneous population of e-EVs in high throughput, and to generate encoded therapeutical microRNAs (i.e., miR-199a-3p and miR-210) in e-EVs. The mRNA-loaded e-EVs were delivered in vivo for cardiac tissue repair in mice with acute myocardial infarction (MI). Treatment using mRNA-loaded e-EVs successfully promoted heart function recovery. As such, this work represents a systematic study proposing a new route to generate and apply EVs in biomedical engineering.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

### BIBLIOGRAPHY

1. Cocucci, E. & Meldolesi, J. Ectosomes and exosomes: shedding the confusion between extracellular vesicles. Trends Cell Biol. 25, 364-372 (2015).
2. Pegtel, D. M. & Gould, S. J. Exosomes. Annu. Rev. Biochem. 88, 487-514 (2019).
3. Alenquer, M. & Amorim, M. Exosome biogenesis, regulation, and function in viral infection. VIRUSES-BASEL 7, 5066-5083 (2015).
4. Kalluri, R. & LeBleu, V. S. The biology, function, and biomedical applications of exosomes. Science 367, eaau6977 (2020).
5. Fruhbeis, C., Fröhlich, D., Kuo, W. P. & Krämer-Albers, E.-M. Extracellular vesicles as mediators of neuron-glia communication. Front. Cell. Neurosci. 7, (2013).
6. Blanchette, C. R. & Rodal, A. A. Mechanisms for biogenesis and release of neuronal extracellular vesicles. Curr. Opin. Neurobiol. 63, 104-110 (2020).
7. Puig, B., Brenna, S. & Magnus, T. Molecular communication of a dying neuron in Stroke. Int. J. Mol. Sci. 19, 2834 (2018).
8. Budnik, V., Ruiz-Cañada, C. & Wendler, F. Extracellular vesicles round off communication in the nervous system. Nat. Rev. Neurosci. 17, 160-172 (2016).
9. Schnatz, A., Müller, C., Brahmer, A. & Krämer-Albers, E. Extracellular vesicles in neural cell interaction and CNS homeostasis. FASEB BioAdvances 3, 577-592 (2021).
10. Song, L., Tian, X. & Schekman, R. Extracellular vesicles from neurons promote neural induction of stem cells through cyclin D1. J. Cell Biol. 220, e202101075 (2021).
11. Fowler, C. D. NeuroEVs: characterizing extracellular vesicles generated in the neural domain. J. Neurosci. 39, 9262-9268 (2019).
12. Xiao, J. et al. Cardiac progenitor cell-derived exosomes prevent cardiomyocytes apoptosis through exosomal miR-21 by targeting PDCD4. Cell Death Dis. 7, (2016).
13. Feng, Y., Huang, W., Wani, M., Yu, X. & Ashraf, M. Ischemic preconditioning potentiates the protective effect of stem cells through secretion of exosomes by targeting Mecp2 via miR-22. PLOS ONE 9, (2014).
14. Mayourian, J. et al. Exosomal microRNA-21-5p mediates mesenchymal stem cell paracrine effects on human cardiac tissue contractility. Circ. Res. 122, 933-944 (2018).
15. Chung, I.-M., Rajakumar, G., Venkidasamy, B., Subramanian, U. & Thiruvengadam, M. Exosomes: current use and future applications. Clin. Chim. Acta 500, 226-232 (2020).
16. Gao, L. et al. Exosomes secreted by hiPSC-derived cardiac cells improve recovery from myocardial infarction in swine. Sci. Transl. Med. 12, eaay1318 (2020).
17. Gao, J., Wang, S. & Wang, Z. High yield, scalable and remotely drug-loaded neutrophil-derived extracellular vesicles (EVs) for anti-inflammation therapy. BIOMATERIALS 135, 62-73 (2017).
18. Shetgaonkar, G. G. et al. Drug Deliv. Transl. Res. 12, 1047-1079 (2022).
19. Ohno, S. & Kuroda, M. Exosome-mediated targeted delivery of miRNAs. in Lentiviral Vectors and Exosomes as Gene and Protein Delivery Tools (ed. Federico, M.) vol. 1448 261-270 (Springer New York, 2016).
20. Lee, K., Yi, Y. & Yu, Y. Remote control of t cell activation using magnetic janus particles. Angew. Chem. Int. Ed. 55, 7384-7387 (2016).
21. Möller, A. & Lobb, R. J. The evolving translational potential of small extracellular vesicles in cancer. Nat. Rev. Cancer 20, 697-709 (2020).
22. Zhang, X. et al. Engineered extracellular vesicles for cancer therapy. Adv. Mater. 33, 2005709 (2021).
23. Herrmann, I. K., Wood, M. J. A. & Fuhrmann, G. Extracellular vesicles as a next-generation drug delivery platform. Nat. Nanotechnol. 16, 748-759 (2021).
24. Lee, J.-H. et al. Emerging biogenesis technologies of extracellular vesicles for tissue regenerative therapeutics. J. Tissue Eng. 12, 204173142110190 (2021).
25. Meng, W. et al. Prospects and challenges of extracellular vesicle-based drug delivery system: considering cell source. Drug Deliv. 27, 585-598 (2020).
26. Sun, L. et al. Serum deprivation elevates the levels of microvesicles with different size distributions and selectively enriched proteins in human myeloma cells in vitro. ACTA Pharmacol. Sin. 35, 381-393 (2014).
27. Burnley-Hall, N., Willis, G., Davis, J., Rees, D. & James, P. Nitrite-derived nitric oxide reduces hypoxia-inducible factor 1 alpha-mediated extracellular vesicle production by endothelial cells. NITRIC OXIDE-Biol. Chem. 63, 1-12 (2017).
28. King, H., Michael, M. & Gleadle, J. Hypoxic enhancement of exosome release by breast cancer cells. BMC CANCER 12, (2012).
29. Yang, Z. et al. Large-scale generation of functional mRNA-encapsulating exosomes via cellular nanoporation. Nat. Biomed. Eng. 4, 69-83 (2020).
30. Najrana, T. et al. Mechanical stretch regulates the expression of specific miRNA in extracellular vesicles released from lung epithelial cells. J. Cell. Physiol. 235, 8210-8223 (2020).
31. Ambattu, L. A. et al. High frequency acoustic cell stimulation promotes exosome generation regulated by a calcium-dependent mechanism. Commun. Biol. 3, 553 (2020).
32. Kumar, R. et al. Fibroblast growth factor 2-mediated regulation of neuronal exosome release depends on VAMP3/cellubrevin in Hippocampal neurons. Adv. Sci. 7, 1902372 (2020).
33. Headland, S., Jones, H., D'Sa, A., Perretti, M. & Norling, L. Cutting-edge analysis of extracellular microparticles using ImageStream(X) imaging flow cytometry. Sci. Rep. 4, (2014).
34. Savina, A., Furlan, M., Vidal, M. & Colombo, M. Exosome release is regulated by a calcium-dependent mechanism in K562 cells. J. Biol. Chem. 278, 20083-20090 (2003).
35. Hsu, C. et al. Regulation of exosome secretion by Rab35 and its GTPase-activating proteins TBC1D10A-C. J. Cell Biol. 189, 223-232 (2010).
36. Ostrowski, M. et al. Rab27a and Rab27b control different steps of the exosome secretion pathway. Nat. Cell Biol. 12, 19-U61 (2010).
37. Trajkovic, K. et al. Ceramide triggers budding of exosome vesicles into multivesicular Endosomes. Science 319, 1244-1247 (2008).
38. Sinha, S. et al. Cortactin promotes exosome secretion by controlling branched actin dynamics. J. Cell Biol. 214, 197-213 (2016).
39. Kojima, R. et al. Designer exosomes produced by implanted cells intracerebrally deliver therapeutic cargo for Parkinson's disease treatment. Nat. Commun. 9, 1305 (2018).
40. Shyong, Y., Chang, K. & Lin, F. Calcium phosphate particles stimulate exosome secretion from phagocytes for the enhancement of drug delivery. Colloids Surf. B-Biointerfaces 171, 391-397 (2018).
41. Messenger, S., Woo, S., Sun, Z. & Martin, T. A Ca²⁺-stimulated exosome release pathway in cancer cells is regulated by Munc13-4. J. Cell Biol. 217, 2877-2890 (2018).
42. Bebelman, M., Bebelman, M., van Niel, G., Pegtel, M. & Verweij, F. Real-time imaging assay of multivesicular body-PM fusion to quantify exosome release from single cells. Protoc. Exch. (2018).
43. Verweij, F. J. et al. Quantifying exosome secretion from single cells reveals a modulatory role for GPCR signaling. J. Cell Biol. 217, 1129-1142 (2018).
49. Eschenhagen, T. et al. Cardiomyocyte regeneration a consensus statement. Circulation 136, 680-686 (2017).
50. Porrello, E. et al. transient regenerative potential of the neonatal mouse heart. Science 331, 1078-1080 (2011).
51. Jopling, C. et al. Zebrafish heart regeneration occurs by cardiomyocyte dedifferentiation and proliferation. Nature 464, 606-U168 (2010).
44. Eulalio, A. et al. Functional screening identifies miRNAs inducing cardiac regeneration. Nature 492, 376-+ (2012).
45. Aguirre, A. et al. In vivo activation of a conserved microrna program induces mammalian heart regeneration. Cell Stem Cell 15, 589-604 (2014).
46. Diez-Cunado, M. et al. miRNAs that induce human cardiomyocyte proliferation converge on the hippo pathway. Cell Rep. 23, 2168-2174 (2018).
50. Giacca, M. & Zacchigna, S. Harnessing the microRNA pathway for cardiac regeneration. J. Mol. Cell. Cardiol. 89, 68-74 (2015).
51. Liu, B. et al. Cell type-specific microRNA therapies for myocardial infarction. Sci. Transl. Med. 13, eabd0914 (2021).
52. Chen, M.-H. et al. MicroRNA-199a regulates myocardial fibrosis in rats by targeting SFRP5. Eur. Rev. Med. Pharmacol. Sci. 23, 3976-3983 (2019).
53. Li, Z. et al. miR-199a impairs autophagy and induces cardiac hypertrophy through mTOR activation. CELL DEATH Differ. 24, 1205-1213 (2017).
54. Tao, Y. et al. miR-199a-3p promotes cardiomyocyte proliferation by inhibiting Cd151 expression. Biochem. Biophys. Res. Commun. 516, 28-36 (2019).
55. Li, Z. et al. miR-199-sponge transgenic mice develop physiological cardiac hypertrophy. Cardiovasc. Res. 110, 258-267 (2016).
56. el Azzouzi, H. et al. The hypoxia-inducible microrna cluster mir-199a similar to 214 targets myocardial PPAR delta and impairs mitochondrial fatty acid oxidation. CELL Metab. 18, 341-354 (2013).
57. Torrini, C. et al. Common regulatory pathways mediate activity of micrornas inducing cardiomyocyte proliferation. Cell Rep. 27, 2759-2771.e5 (2019).
58. Joris, V. et al. MicroRNA-199a-3p and microRNA-199a-5p take part to a redundant network of regulation of the NOS (NO synthase)/NO pathway in the endothelium. Arterioscler. Thromb. Vasc. Biol. 38, 2345-2357 (2018).
59. Fan, Z. et al. MicroRNA-210 promotes angiogenesis in acute myocardial infarction. Mol. Med. Rep. (2018).
60. Wang, N. et al. Mesenchymal stem cells-derived extracellular vesicles, via miR-210, improve infarcted cardiac function by promotion of angiogenesis. Biochim. Biophys. Acta BBA—Mol. Basis Dis. 1863, 2085-2092 (2017).
61. Ahn et al. Manufacturing Therapeutic Exosomes: from Bench to Industry. Mol Cells. 45, (5): 284-290 (2022).
62. Luan et al. Engineering exosomes as refined biological nanoplatforms for drug delivery. Acta Pharmacol Sin. 38, 754-763 (2017).
63. Fukuta et al. Low level electricity increases the secretion of extracellular vesicles from cultured cells. Biochem. Biophys. Rep. 21, 100713 (2019).
64. Chen et al. Exosomes as drug carriers in anti-cancer therapy. Frontiers in Cell and Developmental Biology 10, 728616 (2022).
65. Sun et al. Exosomes as CNS drug delivery tools and their applications. Pharmaceutics 14, 2252 (2022).
66. Comfort et al. Nanoparticle Tracking Analysis for the Quantification and Size Determination of Extracellular Vesicles. J Vis Exp. 169: 10.3791/62447 (2021).

What we claim:

1. A device comprising:
a substrate;
two interdigitated electrodes disposed on the substrate; and
a controller electronically coupled to the two interdigitated electrodes and configured to apply to the two interdigitated electrodes an electrical stimulation sufficient to induce generation of extracellular vesicles from cells disposed on the substrate without killing the cells.

2. The device of claim 1, each of the two interdigitated electrodes comprises a respective plurality of conductive extensions, wherein each of the conductive extensions has a width of between 13 μm and 17 μm and a length greater than 200 μm and is separated from a conductive extension of the opposite electrode by a distance of at least 10 μm.

3. The device of claim 1, wherein the device further comprises a chamber that is coupled to the substrate and that encloses a volume that is exposed to the two interdigitated electrodes.

4. The device of claim 3, wherein the chamber further comprises an inlet for inflow of culture media into the chamber and an outlet for outflow of spent culture media and extracellular vesicles generated by cells disposed within the chamber.

5. The device of claim 1, wherein the electrical stimulation is a biphasic electrical stimulation and wherein the biphasic electrical stimulation comprises repeating biphasic waveforms, wherein each biphasic waveform comprises alternating voltage pulses of a negative pulse and a positive pulse, wherein each pulse has a magnitude between 0.25 V and 1.9 V.

6. The device of claim 5, wherein the biphasic waveforms have a frequency between 0.5 Hz and 10 Hz.

7. The device of claim 6, wherein the voltage pulse lasts for 0.05, 0.1, 0.25, 0.5, 0.75, or 1 seconds.

8. The device of claim 7, wherein the biphasic waveforms occur continuously for a time ranging from 20 seconds, 1, 2, 4, 8, 10, 12, 18, 24, 36, or 48 hours.

9. The device of claim 1, further comprising:
an imager configured to microscopically image cells disposed on the substrate in contact with the two interdigitated electrodes.

10. The device of claim 9, wherein the substrate is optically transparent, and wherein the imager is configured to microscopically image the cells through the substrate.

11. The device of claim 1, wherein the cells release EVs in response to the electrical stimulation.

12. A method of producing extracellular vesicles (EVs) comprising:
culturing a population of cells on electrodes;
stimulating the cells with the electrodes to induce EVs release; and
collecting the released EVs, wherein the cells' plasma membrane remains intact.

13. The method of claim 12, wherein the electrodes comprise interdigitated electrodes.

14. The method of claim 13, wherein the electrical stimulation delivered to the population of cells is a biphasic electrical stimulation.

15. The method of claim 14, wherein the biphasic electrical stimulation comprises repeating biphasic waveforms, wherein each biphasic waveform comprises alternating voltage pulses of a negative pulse and a positive pulse, wherein each pulse has a magnitude between 0.25 V and 1.9 V.

16. The method of claim 15, wherein the biphasic waveforms have a frequency between 0.5 Hz and 10 Hz.

17. The method of claim 15, wherein the voltage pulse lasts for 0.05, 0.1, 0.25, 0.5, 0.75, or 1 seconds.

18. The method of claim 15, wherein the biphasic waveforms occur continuously for a time ranging from 20 seconds, 1, 2, 4, 8, 10, 12, 18, 24, 36, or 48 hours.

19. The method of claim 12, wherein the cells are stem cells, cell lines, primary cells, HeLa cells, or primary cardiac fibroblasts.

20. The method of claim 12, wherein the EVs are exosomes.
